SEQUENCE LISTING (68 sequences)

SEQ ID NO: 1 - protein, 8 aa, synthetic construct
GYTFTNYW

SEQ ID NO: 2 - protein, 8 aa, synthetic construct
VYPGDGDT

SEQ ID NO: 3 - protein, 11 aa, synthetic construct
ARGFTAYGMDY

SEQ ID NO: 4 - protein, 5 aa, synthetic construct
SSVSY

SEQ ID NO: 5 - skipped

SEQ ID NO: 6 - protein, 9 aa, synthetic construct
QQWSSNPLT

SEQ ID NO: 7 - protein, 118 aa, synthetic construct
QVQLQQSGAEVAKPGASVKLSCKASGYTFTNYWMQWIKQAPGQGLEWIGAVYPGDGDTRHTQKFKGKATLTADKSTSTAYMEVSSLRSEDTAVYYCARGFTAYGMDYWGQGTTVTVSS

SEQ ID NO: 8 - protein, 106 aa, synthetic construct
EIVLTQSPATLSLSPGERATLSCSASSSVSYMHWYQQKPGLAPRLLIYDTSKLASGIPDRFSGSGSGTDFTLTISRLEPEDFAVYYCQQWSSNPLTFGQGTKVEIK

SEQ ID NO: 9 - protein, 118 aa, synthetic construct
QVQLQQSGAEVKKPGASVKLSCKASGYTFTNYWMQWIRQAPGQGLEWIGAVYPGDGDTRYTQKFQGRATLTADTSISTAYMEVSRLRSDDTAVYYCARGFTAYGMDYWGQGTTVTVSS

SEQ ID NO: 10 - protein, 106 aa, synthetic construct
EIVLTQSPATMSASPGERVTMSCSASSSVSYMHWYQQKSGLSPKRWIYDTSKLASGVPDRFSGSGSGTDYTFTISRMEPEDFATYYCQQWSSNPLTFGGGTKLEIK

SEQ ID NO: 11 - protein, 118 aa, synthetic construct
QVQLQQSGAEVKKPGASVKLSCKASGYTFTNYWMQWVRQAPGQGLEWIGAVYPGDGDTRYTQKFQGRATLTADTSTSTVYMEVSSLRSEDTAVYYCARGFTAYGMDYWGQGTTVTVSS

SEQ ID NO: 12 - protein, 106 aa, synthetic construct; identical to SEQ ID NO: 10

SEQ ID NO: 13 - protein, 118 aa, synthetic construct
QVQLQQSGAEVKKPGASVKVSCKASGYTFTNYWMQWVRQAPGQGLEWMGAVYPGDGDTRHTQKFKGRVTMTRDTSTSTVYMELSSLRSEDTAVYYCARGFTAYGMDYWGQGTLVTVSS

SEQ ID NO: 14 - protein, 106 aa, synthetic construct
EIVLTQSPATMSASPGERVTMSCSASSSVSYMHWYQQKSGQSPKRWIYDTSKLASGVPARFSGSGSGTDYTFTISSMEPEDFATYYCQQWSSNPLTFGGGTKLEIK

SEQ ID NO: 15 - protein, 118 aa, synthetic construct
QVQLQQSGAEVKKPGASVKVSCKASGYTFTNYWMQWVRQAPGQGLEWIGAVYPGDGDTRHTQKFKGRVTMTADKSTSTVYMELSSLRSEDTAVYYCARGFTAYGMDYWGQGTLVTVSS

SEQ ID NO: 16 - protein, 106 aa, synthetic construct
QIVLTQSPATLSLSPGERATLTCSASSSVSYMHWYQQKPGLSPKRWIYDTSKLASGVPDRFSGSGSGTDYTFTIRRLEPEDFATYYCQQWSSNPLTFGQGTKVEIK

SEQ ID NO: 17 - protein, 118 aa, synthetic construct
QVQLQQSGAEVKKPGASVKLSCKASGYTFTNYWMQWIRQAPGQGLEWIGAVYPGDGDTRYTQKFQGRATLTADTSTSTAYMEVSSLRSEDTAVYYCARGFTAYGMDYWGQGTTVTVSS

SEQ ID NO: 18 - protein, 106 aa, synthetic construct
EIVLTQSPATLSASPGERVTMSCSASSSVSYMHWYQQKPGLAPRRWIYDTSKLASGVPDRFSGSGSGTDYTFTISRMEPEDFATYYCQQWSSNPLTFGGGTKLEIK

SEQ ID NO: 19 - protein, 239 aa, synthetic construct
QVQLQQSGAEVAKPGASVKLSCKASGYTFTNYWMQWIKQAPGQGLEWIGAVYPGDGDTRHTQKFKGKATLTADKSTSTAYMEVSSLRSEDTAVYYCARGFTAYGMDYWGQGTTVTVSSGGGGSGGGGSGGGGSEIVLTQSPATMSASPGERVTMSCSASSSVSYMHWYQQKSGQSPKRWIYDTSKLASGVPARFSGSGSGTDYTFTISSMEPEDFATYYCQQWSSNPLTFGGGTKLEIK

SEQ ID NO: 20 - protein, 239 aa, synthetic construct
QVQLQQSGAEVKKPGASVKLSCKASGYTFTNYWMQWIRQAPGQGLEWIGAVYPGDGDTRYTQKFQGRATLTADTSISTAYMEVSRLRSDDTAVYYCARGFTAYGMDYWGQGTTVTVSSGGGGSGGGGSGGGGSEIVLTQSPATMSASPGERVTMSCSASSSVSYMHWYQQKSGLSPKRWIYDTSKLASGVPDRFSGSGSGTDYTFTISRMEPEDFATYYCQQWSSNPLTFGGGTKLEIK

SEQ ID NO: 21 - protein, 239 aa, synthetic construct
QVQLQQSGAEVKKPGASVKLSCKASGYTFTNYWMQWVRQAPGQGLEWIGAVYPGDGDTRYTQKFQGRATLTADTSTSTVYMEVSSLRSEDTAVYYCARGFTAYGMDYWGQGTTVTVSSGGGGSGGGGSGGGGSEIVLTQSPATMSASPGERVTMSCSASSSVSYMHWYQQKSGLSPKRWIYDTSKLASGVPDRFSGSGSGTDYTFTISRMEPEDFATYYCQQWSSNPLTFGGGTKLEIK

SEQ ID NO: 22 - protein, 242 aa, synthetic construct
QVQLQQSGAEVKKPGASVKVSCKASGYTFTNYWMQWVRQAPGQGLEWMGAVYPGDGDTRHTQKFKGRVTMTRDTSTSTVYMELSSLRSEDTAVYYCARGFTAYGMDYWGQGTLVTVSSASTGGGGSGGGGSGGGGSEIVLTQSPATLSLSPGERATLSCSASSSVSYMHWYQQKPGLAPRLLIYDTSKLASGIPDRFSGSGSGTDFTLTISRLEPEDFAVYYCQQWSSNPLTFGQGTKVEIK

SEQ ID NO: 23 - skipped

SEQ ID NO: 24 - protein, 15 aa, synthetic construct
GGGGSGGGGSGGGGS

SEQ ID NO: 25 - protein, 19 aa, synthetic construct
GGGGSGGGGSGGGSGGGGS

SEQ ID NO: 26 - protein, 24 aa, synthetic construct
GGGGSGGGGSGGGGSGGGSGGGGS

SEQ ID NO: 27 - protein, 29 aa, synthetic construct
GGGGSGGGGSGGGGSGGGGSGGGSGGGGS

SEQ ID NO: 28 - protein, 22 aa, synthetic construct
MDMRVPAQLLGLLLLWLPDTRC

SEQ ID NO: 29 - protein, 19 aa, synthetic construct
MEFGLSWVFLVALLRGVQC

SEQ ID NO: 30 - protein, 118 aa, synthetic construct
QVQLVQSGAEVAKPGASVKLSCKASGYTFTNYWMQWIKQAPGQGLEWIGAVYPGDGDTRHTQKFKGKATLTADKSTSTAYMEVSSLRSEDTAVYYCARGFTAYGMDYWGQGTTVTVSS

SEQ ID NO: 31 - protein, 106 aa, synthetic construct; identical to SEQ ID NO: 14

SEQ ID NO: 32 - protein, 18 aa, synthetic construct
MALPVTALLLPLALLLHA

SEQ ID NO: 33 - protein, 20 aa, synthetic construct
METDTLLLWVLLLWVPGSTG

SEQ ID NO: 34 - protein, 20 aa, synthetic construct
MYRMQLLSCIALSLALVTNS

SEQ ID NO: 35 - protein, 20 aa, synthetic construct
METPAQLLFLLLLWLPDTTG

SEQ ID NO: 36 - protein, 21 aa, synthetic construct
MALPVTALLLPLALLLHAARP

SEQ ID NO: 37 - protein, 16 aa, synthetic construct
MKWVTFISLLFSSAYS

SEQ ID NO: 38 - protein, 30 aa, synthetic construct
MDSKGSSQKGSRLLLLLVVSNLLLCQGVVS

SEQ ID NO: 39 - protein, 118 aa, synthetic construct
QVQLQQSGAELARPGASVKLSCKASGYTFTNYWMQWIKQRPGQGLEWIGAVYPGDGDTRHTQKFKGKATLTADKSSSTAYMQVSSLASEDSAVYYCARGFTAYGMDYWGQGTSVTVSS

SEQ ID NO: 40 - protein, 115 aa, synthetic construct
QVQLQQSGAELARPGASVKXSCKASGYTFTXYWMQWXKQRPGQGLEWIGAXYPGDGDTRXTQKFKGXATLTADKSSSTAYMQXSSLXSEDSAVYYCAXXYXMDYWGQGTSVTVSS

SEQ ID NO: 41 - protein, 106 aa, synthetic construct
QIVLTQSPAVMSASPGEKVTMTCSASSSVSYMHWYQQKSGTSPKRWIYDTSKLASGVPNRFSGSGSGTSYSFTIRSMEAEDAATYYCQQWSSNPLTFGAGTKLELK

SEQ ID NO: 42 - protein, 107 aa, synthetic construct
QIVLTQSPAXMSASPGEKVTMTCSASSSVSYMHWYQQKSGTSPKRWIYDTSKLASGVPXRFSGSGSGTSYSXTIXSMEAEDAATYYCQQWSSNPPLTFGAGTKLELK

SEQ ID NO: 43 - protein, 118 aa, synthetic construct
QVQLQQSGAELARPGASVKLSCKASGYTFTNYWMQWIKQRPGQGLEWIGAVYPGDGDTMMTQKFKGKATLTADKSSSTAYMQVSSLASEDSAVYYCARGFTAYGMDYWGQGTSVTVSS

SEQ ID NO: 44 - protein, 106 aa, synthetic construct; identical to SEQ ID NO: 41

SEQ ID NO: 45 - protein, 5 aa, synthetic construct
GGGGS

SEQ ID NO: 46 - protein, 18 aa, synthetic construct
ASTGGGGSGGGGSGGGGS

SEQ ID NO: 47 - protein, 6 aa, synthetic construct
GSGSGS

SEQ ID NO: 48 - protein, 652 aa, synthetic construct
MDWTWRILFLVAAATGAHSQVQLQQSGAEVAKPGASVKLSCKASGYTFTNYWMQWIKQAPGQGLEWIGAVYPGDGDTRHTQKFKGKATLTADKSTSTAYMEVSSLRSEDTAVYYCARGFTAYGMDYWGQGTTVTVSSGGGGSGGGGSGGGGSEIVLTQSPATMSASPGERVTMSCSASSSVSYMHWYQQKSGQSPKRWIYDTSKLASGVPARFSGSGSGTDYTFTISSMEPEDFATYYCQQWSSNPLTFGGGTKLEIKRAAAIEVMYPPPYLDNEKSNGTIIHVKGKHLCPSPLFPGPSKPKDPKFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 49 - DNA, 8276 bp, synthetic construct
  source 1..8276: mol_type = other DNA
tgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgagtcgcgaccatggactggacctggagaatcctgttcctggtggccgccgctacaggagcccacagccaggtgcagttgcagcagagcggcgctgaggtggccaagcctggagctagcgtgaagctgagctgcaaggccagcggctacaccttcaccaactactggatgcagtggatcaagcaggcccccggacaaggcttggagtggattggcgccgtgtaccccggagatggagacaccaggcacacccagaagttcaagggcaaggccaccctgacagccgacaagagcaccagcaccgcctacatggaggtgagcagcctgaggagcgaggacaccgccgtgtactactgcgccaggggcttcaccgcctacggcatggactattggggccagggcaccacagtgaccgtgagctctggaggaggaggcagcggaggaggaggatctggaggaggcggaagcgagatcgtgctgacccagagccctgccaccatgagcgcttctcccggcgaaagggtgaccatgagctgcagcgccagcagcagcgtgagctacatgcactggtaccagcagaagagcggccagagccccaagaggtggatctacgacaccagcaagctggccagcggcgtgcctgccagattctctggcagcggatctggcaccgactacacattcaccatcagcagcatggagcccgaggacttcgccacctactactgccagcagtggagcagcaaccccctgaccttcggaggcggcaccaagctggagatcaagagggccgccgccatcgaggtgatgtatccccctccctacctggacaacgagaagagcaacggcaccatcatccacgtgaagggcaagcacctgtgccccagccctctgttccctggccctagcaagcccaaggaccccaagttctgggtgctcgtggtggtgggaggagtgctggcctgctacagcctgctggtgaccgtggccttcatcatcttctgggtgctgtgcgccaggcccagaagatctcctgcccaggaggacggcaaggtgtacatcaacatgcccggcaggggcagggtgaagttctctaggagcgctgacgcccctgcttaccagcagggccagaaccagctgtataacgagctgaacctgggcagaagagaggagtacgacgtgctggacaagaggaggggcagggaccccgaaatgggcggcaagcccagaaggaagaacccccaggagggcctgtacaacgagctgcagaaggacaagatggccgaggcctacagcgagatcggcatgaagggcgagaggagaagaggcaagggccacgacggcctgtaccagggcctgagcacagctaccaaggacacctacgacgccctgcacatgcaagcccttccccccagaggaccccagtgcaccaactacgccctgctgaagctggccggcgacgtggaaagcaaccctggccccatgagaatcagcaagccccacctgaggagcatcagcatccagtgctacctgtgcctgctgctgaacagccacttcctgaccgaggccggcatccacgtgttcatcctgggctgcttcagcgccggcctgcccaagaccgaagccaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagctgacaattgcgcgtcatcatcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttttaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaa

SEQ ID NO: 50 - protein, 19 aa, synthetic construct
MDWTWRILFLVAAATGAHS

SEQ ID NO: 51 - protein, 18 aa, synthetic construct
MALPVTALLLPLALLLHA

SEQ ID NO: 52 - protein, 258 aa, synthetic construct
MDWTWRILFLVAAATGAHSQVQLQQSGAEVAKPGASVKLSCKASGYTFTNYWMQWIKQAPGQGLEWIGAVYPGDGDTRHTQKFKGKATLTADKSTSTAYMEVSSLRSEDTAVYYCARGFTAYGMDYWGQGTTVTVSSGGGGSGGGGSGGGGSEIVLTQSPATMSASPGERVTMSCSASSSVSYMHWYQQKSGQSPKRWIYDTSKLASGVPARFSGSGSGTDYTFTISSMEPEDFATYYCQQWSSNPLTFGGGTKLEIK

SEQ ID NO: 53 - protein, 39 aa, synthetic construct
IEVMYPPPYLDNEKSNGTIIHVKGKHLCPSPLFPGPSKP

SEQ ID NO: 54 - protein, 27 aa, synthetic construct
FWVLVVVGGVLACYSLLVTVAFIIFWV

SEQ ID NO: 55 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 24 aa, synthetic construct
LCARPRRSPAQEDGKVYINMPGRG

SEQ ID NO: 57 - protein, 41 aa, synthetic construct
RSKRSRLLHSDYMNMTPRRPGPTRKHYQPYAPPRDFAAYRS

SEQ ID NO: 58 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 59 - DNA, 486 bp, synthetic construct
  source 1..486: mol_type = other DNA
atgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 60 - protein, 22 aa, synthetic construct
GPQCTNYALLKLAGDVESNPGP

SEQ ID NO: 61 - protein, 19 aa, synthetic construct
MEFGLSWLFLVAILKGVQC

SEQ ID NO: 62 - protein, 22 aa, synthetic construct
QNQLYNELNLGRREEYDVLDKR

SEQ ID NO: 63 - protein, 22 aa, synthetic construct
QNQLFNELNLGRREEFDVLDKR

SEQ ID NO: 64 - protein, 23 aa, synthetic construct
QEGLYNELQKDKMAEAYSEIGMK

SEQ ID NO: 65 - protein, 23 aa, synthetic construct
QEGLFNELQKDKMAEAFSEIGMK

SEQ ID NO: 66 - protein, 22 aa, synthetic construct
HDGLYQGLSTATKDTYDALHMQ

SEQ ID NO: 67 - protein, 22 aa, synthetic construct
HDGLFQGLSTATKDTFDALHMQ

SEQ ID NO: 68 - protein, 242 aa, synthetic construct
QVQLQQSGAEVKKPGASVKVSCKASGYTFTNYWMQWVRQAPGQGLEWIGAVYPGDGDTRHTQKFKGRVTMTADKSTSTVYMELSSLRSEDTAVYYCARGFTAYGMDYWGQGTLVTVSSASTGGGGSGGGGSGGGGSQIVLTQSPATLSLSPGERATLTCSASSSVSYMHWYQQKPGLSPKRWIYDTSKLASGVPDRFSGSGSGTDYTFTIRRLEPEDFATYYCQQWSSNPLTFGQGTKVEIK

(19) United States
(12) Patent Application Publication
(10) Pub. No.: US 2024/0180968 A1
(43) Pub. Date: Jun. 6, 2024

(54) ADGRE2 CHIMERIC RECEPTOR NK CELL COMPOSITIONS AND METHODS OF USE

(71) Applicant: Takeda Pharmaceutical Company Limited, Osaka (JP)

(72) Inventors: James Gavin, Cambridge, MA (US); Michael Curley, Cambridge, MA (US); Lan Cao, Cambridge, MA (US)

(21) Appl. No.: 18/494,468

(22) Filed: Oct. 25, 2023

Related U.S. Application Data
(60) Provisional application No. 63/380,896, filed on Oct. 25, 2022.

(51) Int. Cl. A61K 35/17; A61K 39/00; A61P 35/02; C07K 16/28

(52) U.S. Cl. CPC A61K 35/17; A61K 39/4613; A61K 39/4631; A61K 39/464429; A61P 35/02; C07K 16/2896; A61K 2239/13; A61K 2239/21; A61K 2239/22; A61K 2239/38; A61K 2239/48

(57) ABSTRACT

The present application provides cord blood-derived natural killer (CB-NK) cells engineered to express chimeric receptors that target ADGRE2. Pharmaceutical compositions, kits and methods of treating cancer are also provided.

### CROSS-REFERENCE TO RELATED APPLICATIONS

This application claims priority to, and the benefit of U.S. Provisional Patent Application No. 63/380,896 filed on Oct. 25, 2022; the contents of which is incorporated herein by reference in its entirety.

### SEQUENCE LISTING

The instant application contains a Sequence Listing which has been submitted electronically in ST26 (XML) format and is hereby incorporated by reference in its entirety. Said ST26 (XML) copy, created on Oct. 23, 2023, is named MIL-023US1_SL.xml and is 18,127,008 bytes in size.

### BACKGROUND

ADGRE2 antigen binding domains bind to Adhesion G Protein-coupled Receptor E2, ADGRE2 antigen, also known as EMR2, CD312, VBU or CD97. ADGRE2 is expressed by monocytes, macrophages, dendritic cells and granulocytes. ADGRE2 is expressed on cancer cells including acute myeloid leukemia (AML) cells. Cell-based immunotherapy (e.g., chimeric antigen receptor (CAR) T cell therapy) has been shown to be effective in the treatment of certain cancers. However, challenges remain, including systemic cytotoxicity, target antigen negative relapses, and logistic hurdles of generating autologous cell-based products. There remains a need for alternative targets or cell-based immunotherapy targets.

### SUMMARY OF INVENTION

The present invention provides compositions and methods for cell-based immunotherapies for individuals in need thereof, including cell therapies where the cells are genetically modified natural killer (NK) cells (e.g., cord blood-derived natural killer (CB-NK) cells). The modified NK cells express an ADGRE2 chimeric receptor rendering them particularly effective for treatment of autoimmune diseases and cancers, including leukemia, lymphoma, or myeloma, including, for example, relapsed and refractory acute myeloid leukemia.

In one aspect, the present invention provides a cord blood-derived natural killer (CB-NK) cell, comprising a chimeric receptor comprising an extracellular antigen-binding domain that binds to ADGRE2, a transmembrane domain, and an intracellular domain.

In some embodiments, the present invention provides a cord blood-derived natural killer (CB-NK) cell, comprises a chimeric receptor comprising an extracellular antigen-binding domain that binds to ADGRE2, a costimulatory domain and a transmembrane domain.

In some embodiments, the extracellular antigen-binding domain comprises a heavy chain variable region comprises a HCDR1 comprising an amino acid sequence of GYTFTNYW (SEQ ID NO: 1), HCDR2 comprising an amino acid sequence of VYPGDGDT (SEQ ID NO: 2) and HCDR3 comprising an amino acid sequence of ARGFTAYGMDY (SEQ ID NO: 3).

In some embodiments, the extracellular antigen-binding domain comprises a heavy chain variable region that is at least 90%, at least 91%, at least 92%, at least 93%, at least 94%, at least 95%, at least 96%, at least 97%, at least 98%, at least 99%, or 100% identical to the amino acid sequence set forth in SEQ ID NO: 7.

In some embodiments, the extracellular antigen-binding domain comprises a heavy chain variable region that is at least 90%, at least 91%, at least 92%, at least 93%, at least 94%, at least 95%, at least 96%, at least 97%, at least 98%, at least 99%, or 100% identical to the amino acid sequence set forth in SEQ ID NO: 9.

In some embodiments, the extracellular antigen-binding domain comprises a heavy chain variable region that is at least 90%, at least 91%, at least 92%, at least 93%, at least 94%, at least 95%, at least 96%, at least 97%, at least 98%, at least 99%, or 100% identical to the amino acid sequence set forth in SEQ ID NO: 15.

In some embodiments, the extracellular antigen-binding domain comprises a light chain variable region comprises a LCDR1 comprising the amino acid sequence of SSVSY (SEQ ID NO: 4), a LCDR2 comprising an amino acid sequence of DTS (SEQ ID NO: 5), and a LCDR3 comprising an amino acid sequence of QQWSSNPLT (SEQ ID NO: 6).

In one aspect, the present invention provides a cord blood-derived natural killer (CB-NK) cell, comprising a chimeric receptor comprising an extracellular antigen-binding domain that binds to ADGRE2, a transmembrane domain, and an intracellular domain, wherein the extracellular antigen-binding domain comprises a heavy chain variable region comprises a HCDR1 comprising an amino acid sequence of GYTFTNYW (SEQ ID NO: 1), a HCDR2 comprising an amino acid sequence of VYPGDGDT (SEQ ID NO: 2) and a HCDR3 comprising an amino acid sequence of ARGFTAYGMDY (SEQ ID NO: 3); and a light chain variable region comprises a LCDR1 comprising an amino acid sequence of SSVSY (SEQ ID NO: 4), a LCDR2 comprising an amino acid sequence of DTS (SEQ ID NO: 5), and a LCDR3 comprising an amino acid sequence of QQWSSNPLT (SEQ ID NO: 6).

In some embodiments, the extracellular antigen-binding domain comprises a light chain variable region that is at least 90%, at least 91%, at least 92%, at least 93%, at least 94%, at least 95%, at least 96%, at least 97%, at least 98%, at least 99%, or 100% identical to the amino acid sequence set forth in SEQ ID NO: 14.

In some embodiments, the extracellular antigen-binding domain comprises a light chain variable region that is at least 90%, at least 91%, at least 92%, at least 93%, at least 94%, at least 95%, at least 96%, at least 97%, at least 98%, at least 99%, or 100% identical to the amino acid sequence set forth in SEQ ID NO: 12.

In some embodiments, the extracellular antigen-binding domain comprises a light chain variable region that is at least 90%, at least 91%, at least 92%, at least 93%, at least 94%, at least 95%, at least 96%, at least 97%, at least 98%, at least 99%, or 100% identical to the amino acid sequence set forth in SEQ ID NO: 16.

In some embodiments, the extracellular antigen-binding domain comprises a heavy chain variable region that is at least 90%, at least 91%, at least 92%, at least 93%, at least 94%, at least 95%, at least 96%, at least 97%, at least 98%, at least 99%, or 100% identical to the amino acid sequence set forth in SEQ ID NO: 7, and light chain variable region that is at least 90%, at least 91%, at least 92%, at least 93%, at least 94%, at least 95%, at least 96%, at least 97%, at least 98%, at least 99%, or 100% identical to the amino acid sequence set forth in SEQ ID NO: 14.

In some embodiments, the extracellular antigen-binding domain comprises a heavy chain variable region that is at least 90%, at least 91%, at least 92%, at least 93%, at least 94%, at least 95%, at least 96%, at least 97%, at least 98%, at least 99%, or 100% identical to the amino acid sequence set forth in SEQ ID NO: 9, and a light chain variable region that is at least 90%, at least 91%, at least 92%, at least 93%, at least 94%, at least 95%, at least 96%, at least 97%, at least 98%, at least 99%, or 100% identical to the amino acid sequence set forth in SEQ ID NO: 12.

In some embodiments, the extracellular antigen-binding domain comprises a heavy chain variable region that is at least 90%, at least 91%, at least 92%, at least 93%, at least 94%, at least 95%, at least 96%, at least 97%, at least 98%, at least 99%, or 100% identical to the amino acid sequence set forth in SEQ ID NO: 15, and a light chain variable region that is at least 90%, at least 91%, at least 92%, at least 93%, at least 94%, at least 95%, at least 96%, at least 97%, at least 98%, at least 99%, or 100% identical to the amino acid sequence set forth in SEQ ID NO: 16.

In some embodiments, the extracellular antigen-binding domain comprises a single chain variable fragment (scFv).

In some embodiments, the extracellular antigen-binding domain comprises a linker between the heavy chain variable region and the light chain variable region. NO: 24.

In some embodiments, the linker comprises the amino acid sequence of SEQ ID

In some embodiments, the heavy chain variable region and the light chain variable region are positioned from the N- to the C-terminus: VH-VL.

In some embodiments, the extracellular antigen-binding domain comprises an scFv that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence set forth in SEQ ID NO: 19, 20 or 68.

In some embodiments, the extracellular antigen-binding domain comprises an scFv that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence set forth in SEQ ID NO: 19.

In some embodiments, the extracellular antigen-binding domain comprises an scFv that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence set forth in SEQ ID NO: 20.

In some embodiments, the extracellular antigen-binding domain comprises an scFv that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence set forth in SEQ ID NO: 68.

In some embodiments, the transmembrane domain comprises a CD8 polypeptide, a CD28 polypeptide, a CD3s polypeptide, a CD4 polypeptide, a 4-IBB polypeptide, an OX40 polypeptide, an ICOS polypeptide, a CTLA-4 polypeptide, a PD-I polypeptide, a LAG-3 polypeptide, a 2B4 polypeptide, or a BTLA polypeptide.

In some embodiments, the transmembrane domain comprises a CD28 polypeptide. In some embodiments, the transmembrane domain comprises a CD8 polypeptide.

In some embodiments, the intracellular domain further comprises at least one co-stimulatory signaling region.

In some embodiments, the at least one co-stimulatory signaling region comprises a CD28 polypeptide, a 4-1BB polypeptide, an OX40 polypeptide, an ICOS polypeptide, a DAP-10 polypeptide, a DAP-12 polypeptide, a FcϵRIγ polypeptide, a CD160 polypeptide, a NTB-A polypeptide or a combination thereof.

In some embodiments, the at least one co-stimulatory signaling region comprises a CD28 polypeptide or a DAP-10 polypeptide.

In some embodiments, the chimeric receptor is a chimeric antigen receptor (CAR), a chimeric co-stimulating receptor (CCR), or a TCR like fusion molecule.

In some embodiments, the chimeric receptor is a CAR.

In some embodiments, the chimeric receptor is a CAR and the intracellular domain comprises a CD3ξ polypeptide.

In some embodiments, the CD3ξ polypeptide is a modified CD3ξ polypeptide.

In some embodiments, the modified CD3ξ polypeptide comprises a native ITAM1, an ITAM2 variant comprising two loss-of-function mutations, and an ITAM3 variant comprising two loss-of-function mutations.

In some embodiments, the native ITAM1 comprises the amino acid sequence set forth in SEQ ID NO: 63.

In some embodiments, the ITAM2 variant comprises the amino acid sequence set forth in SEQ ID NO: 65.

In some embodiments, the ITAM3 variant comprises the amino acid sequence set forth in SEQ ID NO: 67.

In some embodiments, the modified CD3ξ polypeptide comprises the amino acid sequence set forth in SEQ ID NO: 63.

In some embodiments, the chimeric receptor is a CCR.

In some embodiments, the CB-NK cell further comprises a second chimeric receptor that binds a second target.

In some embodiments, the chimeric receptor that binds ADGRE2 is a chimeric antigen receptor (CAR), and the second chimeric receptor is a chimeric co-stimulatory receptor (CCR).

In some embodiments, the CB-NK cell comprises a chimeric antigen receptor (CAR) that binds ADGRE2 and a second chimeric receptor which is a chimeric antigen receptor (CAR) or a chimeric co-stimulating receptor (CCR). In some embodiments, the CB-NK cell comprises a chimeric co-stimulating receptor (CCR) that binds ADGRE2 and a second chimeric receptor which is a chimeric antigen receptor (CAR) or a chimeric co-stimulating receptor (CCR).

In some embodiments, the second chimeric receptor binds CD123.

In one aspect, the present invention provides a nucleic acid encoding the chimeric receptor.

In some embodiments, the cell is transduced with a nucleic acid encoding the chimeric receptor.

In some embodiments, the chimeric receptor is constitutively expressed on the surface of the cell.

In some embodiments, the nucleic acid further comprises a promoter that is operably linked to the chimeric receptor.

In some embodiments, the promoter is endogenous or exogenous.

In some embodiments, the exogenous promoter is selected from the group consisting of an elongation factor (EF)-1 promoter, a cytomegalovirus immediate-early promoter (CMV) promoter, a simian virus 40 early promoter (SV40) promoter, a phosphoglycerate kinase (PGK) promoter, a metallothionein promoter, and Ubiquitin C promoter.

In some embodiments, the promoter is an inducible promoter.

In some embodiments, the inducible promoter is selected from the group consisting of a NFAT transcriptional response element (TRE) promoter, a CD69 promoter, a CD25 promoter, an IL-2 promoter, a 4-1BB promoter, a PD1 promoter, and a LAG3 promoter.

In some embodiments, the promoter is an endogenous promoter.

In some embodiments, the endogenous promoter is selected from a TCR alpha promoter, a TCR beta promoter, and a beta 2-microglobulin promoter.

In some embodiments, the CB-NK cell further expresses exogenous IL-15.

In one aspect, the present invention provides a composition comprising the CB-NK cell expressing one or more chimeric receptors described herein.

In some embodiments, the pharmaceutical composition further comprising a pharmaceutically acceptable carrier.

In some embodiments, the pharmaceutical composition comprises between about 20×10⁶ and about 150×10⁷ ADGRE2 CB-NK cells.

In some embodiments, the pharmaceutical composition comprises about 20×10⁶, about 100×10⁶ or about 500×10⁶, or about 150×10⁷ ADGRE2 CB-NK cells.

In one aspect, the present invention provides a method of reducing tumor burden in a subject, comprising administering to the subject the ADGRE2 CB-NK cells described herein.

In some embodiments, administering the ADGRE2 CB-NK cells according to the method reduces the number of tumor cells, reduces tumor size, and/or eradicates the tumor in the subject.

In one aspect, the present invention provides a method of increasing or lengthening survival of a subject having a tumor, comprising administering to the subject the ADGRE2 CB-NK cells or compositions comprising the same.

In one aspect, the present invention provides a method of treating and/or preventing a tumor in a subject, comprising administering to the subject the ADGRE2 CB-NK cells or compositions comprising the same.

In some embodiments, the tumor expresses ADGRE2.

In some embodiments, the tumor is cancer.

In some embodiments, the tumor is blood cancer.

In some embodiments, the tumor is selected from the group consisting of multiple myeloma, leukemia, lymphomas, and myeloid malignancies.

In some embodiments, the leukemia is selected from the group consisting of acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia.

In some embodiments, the leukemia is acute myeloid leukemia (AML). (R/R AML).

In some embodiments, the AML is relapsed/refractory acute myeloid leukemia

In some embodiments, the myeloid malignancies are selected from the group consisting of myelodysplastic syndromes (MDS), myeloproliferative neoplasms (MPN), myeloid/lymphoid neoplasms, myeloid/lymphoid neoplasms with eosinophilia and rearrangement of Platelet Derived Growth Factor Receptor Alpha (PDGFRA), Platelet Derived Growth Factor Receptor Beta (PDGFRB), or Fibroblast Growth Factor Receptor 1 (FGFR1), or with PCM1-JAK2), acute myeloid leukemia (AML), blastic plasmacytoid dendritic cell neoplasm, B-lymphoblastic leukemia/lymphoma, and T-lymphoblastic leukemia/lymphoma.

In some embodiments, the myeloid malignancies comprise myelodysplastic syndromes (MDS).

In some embodiments, the subject is a human subject.

In one aspect, the present invention provides a method for producing the CB-NK cells described herein, comprising introducing into the cell a nucleic acid molecule that encodes the chimeric receptor.

In one aspect, the present invention provides a cord blood-derived natural killer (CB-NK) cell, comprising a chimeric receptor comprising an extracellular antigen-binding domain that binds to ADGRE2, a transmembrane domain, and an intracellular domain, wherein the extracellular antigen-binding domain comprises: a heavy chain variable region comprises a HCDR1 comprising an amino acid sequence of GYTFTNYW (SEQ ID NO: 1), a HCDR2 comprising an amino acid sequence of VYPGDGDT (SEQ ID NO: 2) and a HCDR3 comprising an amino acid sequence of ARGFTAYGMDY (SEQ ID NO: 3); and a light chain variable region comprises a LCDR1 comprising an amino acid sequence of SSVSY (SEQ ID NO: 4), a LCDR2 comprising an amino acid sequence of DTS (SEQ ID NO: 5), and a LCDR3 comprising an amino acid sequence of QQWSSNPLT (SEQ ID NO: 6).

In one aspect, the present invention provides a natural killer (NK) cell, comprising a chimeric receptor comprising an extracellular antigen-binding domain that binds to ADGRE2, a transmembrane domain, and an intracellular domain, wherein the extracellular antigen-binding domain comprises: a heavy chain variable region comprises a HCDR1 comprising an amino acid sequence of GYTFTNYW (SEQ ID NO: 1), a HCDR2 comprising an amino acid sequence of VYPGDGDT (SEQ ID NO: 2) and a HCDR3 comprising an amino acid sequence of ARGFTAYGMDY (SEQ ID NO: 3); and a light chain variable region comprises a LCDR1 comprising an amino acid sequence of SSVSY (SEQ ID NO: 4), a LCDR2 comprising an amino acid sequence of DTS (SEQ ID NO: 5), and a LCDR3 comprising an amino acid sequence of QQWSSNPLT (SEQ ID NO: 6), wherein the NK cell further expresses exogenous IL-15.

In some embodiments, the present invention is directed to a cord blood natural killer (CB-NK) cell comprising a nucleic acid encoding a chimeric receptor and an exogenous IL-15 polypeptide, wherein the chimeric receptor comprises:
a) an extracellular antigen-binding domain that binds to ADGRE2 comprising a heavy chain variable region comprises a HCDR1 comprising an amino acid sequence of GYTFTNYW (SEQ ID NO: 1), a HCDR2 comprising an amino acid sequence of VYPGDGDT (SEQ ID NO: 2) and a HCDR3 comprising an amino acid sequence of ARGFTAYGMDY (SEQ ID NO: 3); and
a light chain variable region comprises a LCDR1 comprising an amino acid sequence of SSVSY (SEQ ID NO: 4), a LCDR2 comprising an amino acid sequence of DTS (SEQ ID NO: 5), and a LCDR3 comprising an amino acid sequence of QQWSSNPLT (SEQ ID NO: 6);
b) a hinge domain comprising a CD28 hinge polypeptide
c) a transmembrane domain comprising a CD28 transmembrane polypeptide;
d) a co-stimulatory domain comprising a DAP10 polypeptide; and
e) an intracellular domain is a CD3ξ polypeptide.

In some embodiments, the CD28 hinge polypeptide comprises SEQ ID NO: 53.

In some embodiments, the CD28 transmembrane polypeptide comprises SEQ ID NO: 54.

In some embodiments, the DAP-10 polypeptide comprises SEQ ID NO: 56.

In some embodiments, the CD3ξ polypeptide comprises SEQ ID NO: 55.

In some embodiments, the IL-15 polypeptide comprises SEQ ID NO: 54.

In some embodiments, the present invention is directed to a cord blood natural killer (CB-NK) cell comprising a nucleic acid encoding a chimeric receptor and an exogenous IL-15 polypeptide, wherein the chimeric receptor comprises:
a) an extracellular antigen-binding domain that binds to ADGRE2 comprising a heavy chain variable region comprises a HCDR1 comprising an amino acid sequence of GYTFTNYW (SEQ ID NO: 1), a HCDR2 comprising an amino acid sequence of VYPGDGDT (SEQ ID NO: 2) and a HCDR3 comprising an amino acid sequence of ARGFTAYGMDY (SEQ ID NO: 3); and
a light chain variable region comprises a LCDR1 comprising an amino acid sequence of SSVSY (SEQ ID NO: 4), a LCDR2 comprising an amino acid sequence of DTS (SEQ ID NO: 5), and a LCDR3 comprising an amino acid sequence of QQWSSNPLT (SEQ ID NO: 6),
b) a hinge domain comprising a CD8 hinge polypeptide,
c) a transmembrane domain comprising a CD8 transmembrane polypeptide,
d) a co-stimulatory domain comprising a DAP10 polypeptide, and
e) an intracellular domain is a CD3ξ polypeptide.

In some embodiments, the CD8 hinge polypeptide comprises SEQ ID NO: 69.

In some embodiments, the CD8 transmembrane polypeptide comprises SEQ ID NO: 70.

In some embodiments, the DAP-10 polypeptide comprises SEQ ID NO: 56.

In some embodiments, the CD3ξ polypeptide comprises SEQ ID NO: 55.

In some embodiments, the IL-15 polypeptide comprises SEQ ID NO: 54.

In some embodiments, the present invention is directed to a cord-blood NK cell comprising a chimeric antigen receptor that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence set forth in SEQ ID NO: 48, 72, 75, 76, 79 or 80.

In some embodiments, the chimeric antigen receptor comprises SEQ ID NO: 48.

In some embodiments, the present invention is directed to a cord blood natural killer (CB-NK) cell, comprising a nucleic acid encoding a chimeric receptor and an exogenous IL-15 polypeptide, wherein the chimeric receptor comprises
(a) an extracellular antigen-binding domain that binds to ADGRE2 comprising
a heavy chain variable region comprises a HCDR1 comprising an amino acid sequence of GYTFTNYW (SEQ ID NO: 1), a HCDR2 comprising an amino acid sequence of VYPGDGDT (SEQ ID NO: 2) and a HCDR3 comprising an amino acid sequence of ARGFTAYGMDY (SEQ ID NO: 3); and
a light chain variable region comprises a LCDR1 comprising an amino acid sequence of SSVSY (SEQ ID NO: 4), a LCDR2 comprising an amino acid sequence of DTS (SEQ ID NO: 5), and a LCDR3 comprising an amino acid sequence of QQWSSNPLT (SEQ ID NO: 6);
(b) a hinge domain comprising a CD8 polypeptide;
(c) a transmembrane domain comprising a CD8 polypeptide; and
(d) a co-stimulatory domain comprising an OX-40 polypeptide.

In some embodiments, the CD8 hinge polypeptide comprises SEQ ID NO: 69.

In some embodiments, the CD8 transmembrane polypeptide comprises SEQ ID NO: 70.

In some embodiments, the present invention is directed to a cord blood natural killer (CB-NK) cell, comprising a nucleic acid encoding a chimeric receptor and an exogenous IL-15 polypeptide, wherein the chimeric receptor comprises
(a) an extracellular antigen-binding domain that binds to ADGRE2 comprising
a heavy chain variable region comprises a HCDR1 comprising an amino acid sequence of GYTFTNYW (SEQ ID NO: 1), a HCDR2 comprising an amino acid sequence of VYPGDGDT (SEQ ID NO: 2) and a HCDR3 comprising an amino acid sequence of ARGFTAYGMDY (SEQ ID NO: 3); and
a light chain variable region comprises a LCDR1 comprising an amino acid sequence of SSVSY (SEQ ID NO: 4), a LCDR2 comprising an amino acid sequence of DTS (SEQ ID NO: 5), and a LCDR3 comprising an amino acid sequence of QQWSSNPLT (SEQ ID NO: 6);
(b) a hinge domain comprising a CD28 polypeptide;
(c) a transmembrane domain comprising a CD28 polypeptide; and
(d) a co-stimulatory domain comprising an OX-40 polypeptide.

In some embodiments, the CD28 hinge polypeptide comprises SEQ ID NO: 53.

In some embodiments, the CD28 transmembrane polypeptide comprises SEQ ID NO: NO: 54.

In some embodiments, the OX40 polypeptide comprises SEQ ID NO: 71.

In some embodiments, the IL-15 polypeptide comprises SEQ ID NO: 54.

In some embodiments, the present invention is directed to a cord-blood cell comprising a chimeric antigen receptor that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence set forth in SEQ ID NO: 73, 74, 77, 78, 81 or 82.

In some embodiments, the present invention provides a nucleic acid encoding a chimeric receptor and an exogenous IL-15 polypeptide, wherein the chimeric receptor comprises an extracellular antigen-binding domain that binds to ADGRE2 comprising a heavy chain variable region comprises a HCDR1 comprising an amino acid sequence of GYTFTNYW (SEQ ID NO: 1), a HCDR2 comprising an amino acid sequence of VYPGDGDT (SEQ ID NO: 2) and a HCDR3 comprising an amino acid sequence of ARGFTAYGMDY (SEQ ID NO: 3); and a light chain variable region comprises a LCDR1 comprising an amino acid sequence of SSVSY (SEQ ID NO: 4), a LCDR2 comprising an amino acid sequence of DTS (SEQ ID NO: 5), and a LCDR3 comprising an amino acid sequence of QQWSSNPLT (SEQ ID NO: 6).

### Definitions

A or An: The articles “a” and “an” are used herein to refer to one or to more than one (i.e., to at least one) of the grammatical object of the article. By way of example, “an element” means one element or more than one element.

Affinity: As used herein, the term “affinity” refers to the characteristics of a binding interaction between a binding moiety (e.g., an antigen binding moiety (e.g., variable domain described herein) and/or Fc receptor binding moiety (e.g., FcRn binding moiety described herein)) and a target (e.g., an antigen (e.g., ADGRE2) and/or FcR (e.g., FcRn)) and that indicates the strength of the binding interaction. In some embodiments, the measure of affinity is expressed as a dissociation constant (K_{D}). In some embodiments, a binding moiety has a high affinity for a target (e.g., a K_{D} of less than about 10⁻⁷ M, less than about 10⁻⁸ M, or less than about 10⁻⁹ M). In some embodiments, a binding moiety has a low affinity for a target (e.g., a K_{D} of higher than about 10⁻⁷ M, higher than about 10⁻⁶ M, higher than about 10⁻⁵ M, or higher than about 10⁻⁴ M). In some embodiments, a binding moiety has high affinity for a target at a first pH, has low affinity for the target at a second pH, and has an intermediate affinity for the target at a pH level between the first pH and the second pH.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab, F(ab′)₂, Fd, Fv, and dAb fragments) as well as complete antibodies, e.g., intact immunoglobulins of types IgA, IgG, IgE, IgD, IgM (as well as subtypes thereof). The light chains of the immunoglobulin can be of types kappa or lambda.

Binding Moiety: As used herein, a “binding moiety” is any molecule or part of a molecule capable of specifically binding a target, e.g., a target of interest (e.g., an antigen (e.g., ADGRE2) and/or FcR (e.g., FcRn)). Binding moieties include, e.g., antibodies, antigen-binding fragments thereof, Fc regions or Fc fragments thereof, antibody mimetics, peptides, and aptamers.

Antigen-binding fragment or antibody fragment thereof refers to a portion of an intact antibody. An antigen-binding fragment or antibody fragment thereof refers to a portion of an intact antibody that binds to an antigen (e.g., ADGRE2). An antigen-binding fragment can contain the antigenic determining variable regions of an intact antibody. Examples of antibody fragments include, but are not limited to Fab, Fab′, F(ab′)₂, and Fv fragments, linear antibodies, antibody mimetics, scFvs, and single chain antibodies.

Complementarity Determining Region (CDR): A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283: 1 156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

Constant region: As used herein, the term “constant region” refers to a polypeptide that corresponds to, or is derived from, one or more constant region immunoglobulin domains of an antibody. A constant region can include any or all of the following immunoglobulin domains: a CH1 domain, a hinge region, a CH2 domain, a CH3 domain (derived from an IgA, IgD, IgG, IgE, or IgM), and a CH4 domain (derived from an IgE or IgM).

Epitope: As used herein, an “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giege R et al, (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antibody:antigen crystals may be studied using well known X-ray diffraction techniques and may be refined using computer software known in the art, e.g., Refmac and Phenix. Mutagenesis mapping studies may be accomplished using any method known to one of skill in the art. See, e.g., Champe M et al, (1995) J Biol Chem 270: 1388-1394 and Cunningham BC & Wells J A (1989) Science 244: 1081-1085 for a description of mutagenesis techniques, including alanine scanning mutagenesis techniques.

Fc region: As used herein, the term “Fc region” refers to a dimer of two “Fc polypeptides”, each “Fc polypeptide” comprising the constant region of an antibody excluding the first constant region immunoglobulin domain. In some embodiments, an “Fc region” includes two Fc polypeptides linked by one or more disulfide bonds, chemical linkers, or peptide linkers. “Fc polypeptide” refers to the last two constant region immunoglobulin domains of IgA, IgD, and IgG, and the last three constant region immunoglobulin domains of IgE and IgM, and may also include part or all of the flexible hinge N-terminal to these domains. For IgG, “Fc polypeptide” comprises immunoglobulin domains Cgamma2 (Cγ2) and Cgamma3 (Cγ3) and the lower part of the hinge between Cgammal (Cγ1) and C∛2. Although the boundaries of the Fc polypeptide may vary, the human IgG heavy chain Fc polypeptide is usually defined to comprise residues starting at T223 or C226 or P230, to its carboxyl-terminus, wherein the numbering is according to the EU index as in Kabat et al. (1991, NIH Publication 91-3242, National Technical Information Services, Springfield, VA). For IgA, Fc polypeptide comprises immunoglobulin domains Calpha2 (Cα2) and Calpha3 (Cα3) and the lower part of the hinge between Calpha1 (Cα1) and Cα2. An Fc region can be synthetic, recombinant, or generated from natural sources such as IVIG.

Genetically engineered or genetically modified: As used herein, the term “genetically engineered” or “genetically modified” refers to the addition of extra genetic material in the form of DNA or RNA, such as DNA or RNA encoding a ADGRE2 targeting CAR polypeptide of the present invention, into the total genetic material in a cell. he terms, “genetically modified cells,” “modified cells,” and “cells that are genetically engineered or modified to express,” are used interchangeably.

Humanized antibodies: As used herein, humanized antibodies are antibodies derived from non-human species whose protein sequences have been modified to increase their similarity to antibody variants produced naturally in humans. “Humanization” is usually applied to monoclonal antibodies developed for administration to humans (for example, anti-ADGRE2 antibodies developed as anti-cancer drugs). In some embodiments, humanization is employed when developing a specific antibody in a non-human immune system (such as that in mice).

Kₐ: As used herein, “Kₐ” refers to an association rate of a particular binding moiety and a target to form a binding moiety/target complex.

K_{d}: As used herein, “K_{d}” refers to a dissociation rate of a particular binding moiety/target complex.

K_{D}: As used herein, “K_{D}” refers to a dissociation constant, which is obtained from the ratio of K_{d} to Kₐ (i.e., K_{d}/Kₐ) and is expressed as a molar concentration (M). K_{D} values can be determined using methods well established in the art, e.g., by using surface plasmon resonance, or using a biosensor system such as a Biacore® system.

Reference: A “reference” entity, system, amount, set of conditions, etc., is one against which a test entity, system, amount, set of conditions, etc. is compared as described herein. For example, in some embodiments, a “reference” antibody is a control antibody that is not engineered as described herein.

Selective binding: As used herein, “selective binding”, “selectively binds” “specific binding”, or “specifically binds” refers, with respect to a binding moiety and a target, preferential association of a binding moiety to a target and not to an entity that is not the target. A certain degree of non-specific binding may occur between a binding moiety and a non-target. In some embodiments, a binding moiety selectively binds a target if binding between the binding moiety and the target is greater than 2-fold, greater than 5-fold, greater than 10-fold, or greater than 100-fold as compared with binding of the binding moiety and a non-target. In some embodiments, a binding moiety selectively binds a target if the binding affinity is less than about 10⁻⁵ M, less than about 10⁻⁶ M, less than about 10⁻⁷ M, less than about 10⁻⁸ M, or less than about 10⁻⁹ M. In some embodiments, a molecule that specifically binds to an antigen may bind to other peptides or polypeptides, generally with lower affinity as determined by, e.g., immunoassays, BIACORE®, KinExA 3000 instrument (Sapidyne Instruments, Boise, ID), or other assays known in the art.

Single-chain variable fragment (scFv): As used herein, the term “single-chain variable fragment” or “scFv” refers to a fusion protein of the variable regions of the heavy (V_{H}) and light chains (V_{L}) of an immunoglobulin (e.g., mouse or human) covalently linked to form a V_{H}::VL heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties.

Subject: The term “subject”, as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

Target: As used herein, a “target” is any molecule specifically bound by a binding moiety of an antibody or an antigen-binding fragment thereof. In some embodiments, a target is an antigen described herein (e.g., ADGRE2). The terms “first target” and “second target” are used herein to refer to molecules of two distinct molecular species, rather than two molecules of the same molecular species. For example, in some embodiments, a first target is ADGRE2 and a second target is CD123.

Therapeutically effective amount: As used herein, the term “therapeutically effective amount” refers to an amount of a therapeutic molecule (e.g., an ADGRE2 antigen binding domain described herein) which confers a therapeutic effect on a treated subject, at a reasonable benefit/risk ratio applicable to any medical treatment. Therapeutic effect may be objective (i.e., measurable by some test or marker) or subjective (i.e., subject gives an indication of or feels an effect). In particular, the “therapeutically effective amount” refers to an amount of a therapeutic molecule or composition effective to treat, ameliorate, or prevent a particular disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount can be administered in a dosing regimen that may comprise multiple unit doses. For any particular therapeutic molecule, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific pharmaceutical agent employed; the specific composition employed; the age, body weight, general health, sex and diet of the subject; the time of administration, route of administration, and/or rate of excretion or metabolism of the specific therapeutic molecule employed; the duration of the treatment; and like factors as is well known in the medical arts.

Treatment: As used herein, the term “treatment” (also “treat” or “treating”) refers to any administration of a therapeutic molecule (e.g., an ADGRE2 antigen binding domain described herein) that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

### BRIEF DESCRIPTION OF DRAWINGS

Drawings are for illustration purposes only; not for limitation.

FIG. 1A-1C shows exemplary results of in vitro cytotoxicity activity of ADGRE2 CAR or CCR CB-NK against acute myeloid leukemia cell lines MOLM-13 (FIG. 1A), KG-1a (FIG. 1B), and MV-4-11 (FIG. 1C).

FIG. 2A-2C shows exemplary results of in vivo efficacy of ADGRE2 CAR CB-NK against acute myeloid leukemia cell lines MOLM-13 (FIG. 2A), KG-la (FIG. 2B), and MV-4-11 (FIG. 2C).

FIG. 3A shows exemplary co-stimulatory domains for chimeric receptors that were probed in MOLM-13 killing assays. FIG. 3B and FIG. 3C exemplify the ability of NK cells engineered with CD123-OX40-CCR to durably control MOLM-13 tumor cells across many different tumor re-challenges in two different donors—donor A (FIG. 3B) and donor B (FIG. 3C).

### DETAILED DESCRIPTION

The present disclosure is based, in part, on the discovery that CB-NK cells engineered to express a chimeric receptor that binds to Adhesion G Protein-Coupled Receptor E2, ADGRE2 (e.g., human ADGRE2) demonstrate target specific cytotoxicity.

ADGRE2, also known as EMR2, CD312, VBU or CD97, is a cell surface receptor that is a member of the adhesion G-protein coupled receptor (GPCR) family. ADGRE2 binds to the chondroitin sulfate moiety of glycosaminoglycan chains and promotes cell attachment. ADGRE2 is expressed by monocytes, macrophages, dendritic cells and granulocytes playing a role in chemotaxis, cell adhesion and degranulation. In macrophages, ADGRE2 promotes the release of inflammatory cytokines, including IL8 and TNF, signaling through G-proteins.

ADGRE2 is highly expressed in cancerous cells, including for example in relapsed and refractory acute myeloid leukemia. In some embodiments, the ADGRE2 CB-NK cells are used to treat cancers. In some embodiments, ADGRE2 CB-NK cells are used to treat relapsed and refractory acute myeloid leukemia.

### Cells

In one aspect, the invention provides a cord-blood natural killer (CB-NK) cell genetically engineered to express a ADGRE2 targeting chimeric receptor (e.g., a CAR or CCR polypeptide described herein). CB-NK cells are immune effector cells. An “immune effector cell,” is any cell of the immune system that has one or more effector functions (e.g., cytotoxic cell killing activity, secretion of cytokines, induction of ADCC and/or CDC). As described herein, the CB-NK cell is transformed with a polynucleotide encoding the chimeric receptor (e.g., CAR or CCR construct) such that the chimeric receptor is expressed on the cell surface. The CB-NK cells described herein exhibit an antitumor property.

Methods for making the CB-NK cells that express a chimeric receptor contemplated herein are provided. In one embodiment, the method comprises transfecting or transducing CB-NK cells isolated from an individual such that the CB-NK cells express one or more chimeric receptors contemplated herein. In certain embodiments, the CB-NK cells are isolated from an individual and genetically modified without further manipulation in vitro. Such cells can then be directly readministered into the individual. In further embodiments, the CB-NK cells are first activated and stimulated to proliferate in vitro prior to being genetically modified to express a chimeric receptor. In this regard, the CB-NK cells may be cultured before and/or after being genetically modified (i.e., transduced or transfected to express a ADGRE2 targeting chimeric receptor contemplated herein).

For example, in some embodiments, the CB-NK cells are transduced with a viral vector encoding a chimeric receptor polypeptide of the present invention. In some embodiments, the viral vector is a retroviral vector, such as a lentiviral vector and an AAV vector. In other examples, the CB-NK cell is transfected with a nucleic acid molecule, e.g., mRNA, cDNA, DNA, encoding a chimeric receptor polypeptide of the present invention.

### Sources of Cells

In accordance with the present invention, in some embodiments, immune effector cells are obtained from a subject for expansion and genetic modification to express the chimeric receptor polypeptides of the present invention.

Immune effector cells can be autologous/autogenic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells are autologous.

In some embodiments, immune effector cells are NK cells. NK cells may be derived from cord blood, peripheral blood, induced pluripotent stem cells, hematopoietic stem cells, bone marrow. The NK cell may be derived from a cord blood mononuclear cell. The NK cell may be a CD56+ NK cell.

In some aspects, any immune effector cell lines available in the art, may be used. For example, NK cells may be derived from the NK-92 cell line.

In some embodiments, cells transduced with a polynucleotide encoding the chimeric receptor construct as described herein are expanded. In some embodiments, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days).

In some embodiments, the cells are expanded in an appropriate media that includes one or more interleukin that result in at least a 100-fold, at least a 150-fold, at least a 200-fold (e.g., 200-fold, 250-fold, 300-fold, 350-fold) increase in cells over a 14-day expansion period, e.g., as measured by a method such as flow cytometry.

In some embodiments, a population of modified immune effector cells for the treatment of cancer comprises a ADGRE2 targeting chimeric receptor contemplated herein. For example, a population of modified immune effector cells a population of NK cells.

### Chimeric Receptors

In one aspect, the present invention relates to cells expressing ADGRE2 targeting chimeric receptor polypeptides and polynucleotides encoding the same. The ADGRE2 targeting chimeric receptor comprises an extracellular antigen binding region that specifically binds to ADGRE2, a hinge domain, a transmembrane domain, and a costimulatory domain. In some embodiments, the chimeric receptor further comprises at least one intracellular activation/signaling domain. The ADGRE2 targeting chimeric receptor may further comprise one or more additional polypeptides such as a cytokine (e.g., IL-15). Each component within the chimeric receptor may be linked by one or more linker sequences.

In some embodiments, the ADGRE2 targeting chimeric receptor is a chimeric antigen receptor (CAR). In some embodiments, the ADGRE2 targeting chimeric receptor is a chimeric co-stimulating receptor (CCR). In some embodiments, the chimeric receptor is a TCR like fusion molecule.

### Chimeric Antigen Receptor (CAR)

In certain embodiments, the chimeric receptor is a CAR. CARs are engineered receptors, which graft or confer a specificity of interest onto an immune effector cell (e.g., a CB-NK cell). In some embodiments, the CAR is composed of an extracellular antigen-binding domain (e.g., an scFv), which is fused to a transmembrane domain, which is fused to cytoplasmic/intracellular signaling domain. In some embodiments, the CAR further comprises intracellular signaling domains from a co-stimulatory molecule (e.g., CD28, DAP10, 4-1BB, ICOS, OX40). In some embodiments, the CAR comprises a co-stimulatory molecule. In some embodiments, the co-stimulatory molecule is CD28 or DAP10. In some embodiments, the co-stimulatory molecule is CD28. In some embodiments, the co-stimulatory molecule is DAP10. When the CAR comprises a co-stimulatory domain, additional signals are added to the effector cell. In some embodiments, the CAR comprises both co-stimulation (e.g., CD28 or 4-IBB) and activation (CD3ξ). In some embodiments, the CAR comprises multiple co-stimulation (e.g., CD28 and 4-IBB) domains and activation (CD3ξ) signals.

### Chimeric Co-Stimulatory Receptor (CCR)

In certain embodiments, the chimeric receptor is a CCR. The presently disclosed CCR binds to an antigen (e.g., ADGRE2) and provides co-stimulatory signals, but does not alone provide an activation signal. In certain embodiments, the CCR does not comprise a CD3ξ polypeptide. CCRs provide co-stimulation, e.g., a CD28-like signal, in the absence of the natural co-stimulatory ligand on the antigen-presenting cell. A combinatorial antigen recognition, i.e., use of a CCR in combination with a CAR, can augment T-cell reactivity against the dual-antigen expressing T cells, thereby improving selective tumor targeting. Kloss et al., describe a strategy that integrates combinatorial antigen recognition, split signaling, and, critically, balanced strength of T-cell activation and co-stimulation to generate T cells that eliminate target cells that express a combination of antigens while sparing cells that express each antigen individually (Kloss et al., Nature Biotechnology (2013);31(1):71-75, the content of which is incorporated by reference in its entirety). With this approach, T-cell activation requires CAR-mediated recognition of one antigen, whereas co-stimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen.

In certain embodiments, the CCR comprises an extracellular antigen-binding domain that binds to an antigen (e.g., ADGRE2), a transmembrane domain, and a co-stimulatory signaling region that comprises an intracellular domain of at least one co-stimulatory molecule or a fragment thereof. In certain embodiments, the CCR does not alone deliver an activation signal to an immunoresponsive cell. Non-limiting examples of co-stimulatory molecules include CD28, 4-1BB, OX40, ICOS, DAP-10, CD27, CD40, and NKGD2. In certain embodiments, the co-stimulatory signaling region of the CCR comprises an intracellular domain of a co-stimulatory signaling molecule or a fragment thereof. In certain embodiments, the one co-stimulatory signaling molecule is CD28. In certain embodiments, the one co-stimulatory signaling molecule is 4-1BB. In certain embodiments, the one co-stimulatory signaling molecule is OX40. In certain embodiments, the co-stimulatory signaling region of the CCR comprises an intracellular domain of a first co-stimulatory signaling molecule or a fragment thereof and an intracellular domain of a second co-stimulatory signaling molecule or a fragment thereof. In certain embodiments, the first and second co-stimulatory signaling molecules are CD28 and 4-1BB.

Similar to a CAR, the extracellular antigen-binding domain of the CCR can be an scFv, a Fab, a F(ab)2, or a fusion protein with a heterologous sequence to form the extracellular antigen binding domain of the CCR.

TCR like Fusion Molecules

In certain embodiments, the chimeric receptor is a TCR like fusion molecule. Nonlimiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), T cell receptor fusion constructs (TRuCs) (e.g., those disclosed in Baeuerle et al., “Synthetic TruC receptors engaging the complete T cell receptor for potent anti-tumor response,” Nature Communications volume 10, Article number: 2087 (2019), which is incorporated by reference in its entirety), synthetic T cell receptor and antigen receptor (STAR) (e.g., those disclosed in Liu et al. Science Translational Medicine (2021);13(586):eabb5191, which is incorporated by reference in its entirety), antibody-T-cell receptor (AbTCR) (e.g., those disclosed in Xu et al. Cell Discovery (2018) 4:62, which is incorporated by reference in its entirety), and T cell antigen coupler (TAC) (e.g., those disclosed in Helsen et al. Nature Communications (2018);9:3049, which is incorporated by reference in its entirety).

In certain embodiments, the TCR like fusion molecule comprises an antigen binding chain that comprises an extracellular antigen-binding domain and a constant domain, wherein the TCR like fusion molecule binds to an antigen in an HLA-independent manner. In certain embodiments, the constant domain comprises a T cell receptor constant region selected from the group consisting of a native or modified TRAC polypeptide, a native or modified TRBC polypeptide, a native or modified TRDC polypeptide, a native or modified TRGC polypeptide and any variants or functional fragments thereof. In certain embodiments, the constant domain comprises a native or modified TRAC polypeptide. In certain embodiments, the constant domain comprises a native or modified TRBC polypeptide. In certain embodiments, the constant domain is capable of forming a homodimer or a heterodimer with another constant domain. In certain embodiments, the antigen binding chain is capable of associating with a CD3ξ polypeptide. In certain embodiments, the antigen binding chain, upon binding to an antigen (e.g., ADGRE2), is capable of activating the CD3ξ polypeptide associated to the antigen binding chain. In certain embodiments, the activation of the CD3ξ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR.

In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (VH) of an antibody. In certain embodiments, the extracellular antigen-binding domain of the TCR like fusion molecule comprises a light chain variable region (VL) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a VH of an antibody, wherein the VH is capable of dimerizing with another extracellular antigen-binding domain comprising a VL of the antibody and form a fragment variable (Fv). In certain embodiments, the extracellular antigen-binding domain of the TCR like fusion molecule comprises a VL of an antibody, wherein the VL is capable of dimerizing with another extracellular antigen-binding domain comprising a VH of the antibody and form a fragment variable (Fv).

### ADGRE2 Antigen Binding Domains

The antigen binding domain of the chimeric receptors described herein specifically bind to ADGRE2 antigen. In some embodiments, the anti-ADGRE2 chimeric receptors binds human ADGRE2. ADGRE2 is described in the art, for example, in Uniprot sequence reference Q9UHX3-1. In some embodiments, human ADGRE2 comprises an EGF-like 1 domain, an EGF-like 2 domain, an EGF-like 3 domain, an EGF-like 4 domain, an EGF-like 5 domain, and a GPS domain.

The ADGRE2 binding domain can be any protein that binds to ADGRE2 or a portion of ADGRE2 (e.g., an ADGRE2 binder). The ADGRE2 binding domain may be an antibody that specifically binds to human ADGRE2, or antigen binding fragment thereof. The antibody or antigen-binding fragment that specifically binds to ADGRE2 may be a monoclonal antibody; a monospecific antibody; a humanized antibody; a human antibody; a single chain antibody; a domain-specific antibody; a single domain antibody; a domain-deleted antibody; an scFc fusion protein; a single-chain antibody; a chimeric antibody; a synthetic antibody; a recombinant antibody; a hybrid antibody; a mutated antibody, a CDR-grafted antibody; an antibody fragment such as an Fab; an Ftab\hat{ } fragment; an Fab′ fragment; an F(ab)2 fragment; a Fv fragment; a single-chain Fv (scFv) fragment; an Fd fragment; a dAb fragment; a diabody; a nanobody; a bivalent nanobody; a shark variable IgNAR domain; a VHH antibody; a camelid antibody; and a minibody.

In some embodiments, a suitable heavy chain constant region is derived from IgG1 or IgG4. A light chain can include a light chain variable domain and a light chain constant domain. A light chain constant domain can include either a kappa light chain or a lambda light chain. A heavy chain variable domain of a heavy chain and a light chain variable domain of a light chain can typically be further subdivided into regions of variability, termed complementarity determining regions (CDRs), interspersed with regions that are more conserved, termed framework regions (FR). Such heavy chain and light chain variable domains can each include three CDRs and four framework regions, arranged from amino-terminus to carboxyl-terminus in the following order: FR1, CDR1, FR2, CDR2, FR3, CDR3, FR4, one or more of which can be engineered as described herein. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989). As used herein, CDRs are referred to for each of the heavy (HCDR1, HCDR2, HCDR3) and light (LCDR1, LCDR2, LCDR3) chains.

In some embodiments, the ADGRE2 binder comprises three heavy chain complementarity determining regions (HCDRs), i.e., HCDR1, HCDR2 and HCDR3 in the heavy chain variable region (VH), and/or three light chain complementarity determining regions (LCDRs), i.e., LCDR1, LCDR2 and LCDR3, in the light chain variable region (VL). In some embodiments, the ADGRE2 binder comprises a VH and/or a VL.

In some embodiments, the anti-ADGRE2 scFv comprises the amino acid sequence set forth in SEQ ID NO: 19, SEQ ID NO: 20, SEQ ID NO: 21, SEQ ID NO: 22 or SEQ ID NO: 68. In some embodiments, the anti-ADGRE2 scFv comprises SEQ ID NO: 19.

In some embodiments, the anti-ADGRE2 scFv comprises a VH and a VL that is disclosed in WO2017/087800, which is incorporated by reference in its entirety.

Embodiments of the invention include antibodies and antigen binding fragments comprising the CDRs found in the VH and VL domains described herein that are identified using conventional numbering systems, such as the IMGT, Kabat and Chothia numbering systems. Such numbering systems are well-known in the art. In certain embodiments, the CDRs are identified or numbered according to the IMGT numbering system.

### Exemplary ADGRE2 Antigen-Binding Domains

In some embodiments, the ADGRE2 antigen binding domain or fragments thereof described herein comprises heavy chain variable region (VH) complementarity determining region (CDR) sequences: VH CDR1: GYTFTNYW (SEQ ID NO: 1), VH CDR2: VYPGDGDT (SEQ ID NO: 2) and VH CDR3: ARGFTAYGMDY (SEQ ID NO: 3).

In some embodiments, the heavy chain variable region comprises an amino acid sequence of

(SEQ ID NO: 7)
QVQLQQSGAEVAKPGASVKLSCKASGYTFTNYWMQWIKQA
PGQGLEWIGAVYPGDGDTRHTQKFKGKATLTADKSTSTAY
MEVSSLRSEDTAVYYCARGFTAYGMDYWGQGTTVTVSS.

In some embodiments, the heavy chain variable region comprises an amino acid sequence of

(SEQ ID NO: 9)
QVQLQQSGAEVKKPGASVKLSCKASGYTFTNYWMQWIRQA
PGQGLEWIGAVYPGDGDTRYTQKFQGRATLTADTSISTAY
MEVSRLRSDDTAVYYCARGFTAYGMDYWGQGTTVTVSS.

In some embodiments, the heavy chain variable region comprises an amino acid sequence of

(SEQ ID NO: 11)
QVQLQQSGAEVKKPGASVKLSCKASGYTFTNYWMQWVRQA
PGQGLEWIGAVYPGDGDTRYTQKFQGRATLTADTSTSTVY
MEVSSLRSEDTAVYYCARGFTAYGMDYWGQGTTVTVSS.

In some embodiments, the heavy chain variable region comprises an amino acid sequence of

(SEQ ID NO: 13)
QVQLQQSGAEVKKPGASVKVSCKASGYTFTNYWMQWVRQA
PGQGLEWMGAVYPGDGDTRHTQKFKGRVTMTRDTSTSTVY
MELSSLRSEDTAVYYCARGFTAYGMDYWGQGTLVTVSS.

In some embodiments, the heavy chain variable region comprises an amino acid sequence of

(SEQ ID NO: 15)
QVQLQQSGAEVKKPGASVKVSCKASGYTFTNYWMQWVRQA
PGQGLEWIGAVYPGDGDTRHTQKFKGRVTMTADKSTSTVY
MELSSLRSEDTAVYYCARGFTAYGMDYWGQGTLVTVSSAS
T.

In some embodiments, the heavy chain variable region comprises an amino acid sequence of

(SEQ ID NO: 17)
QVQLQQSGAEVKKPGASVKLSCKASGYTFTNYWMQWIRQA
PGQGLEWIGAVYPGDGDTRYTQKFQGRATLTADTSTSTAY
MEVSSLRSEDTAVYYCARGFTAYGMDYWGQGTTVTVSS.

In some embodiments, the heavy chain variable region comprises an amino acid sequence of

(SEQ ID NO: 30)
QVQLVQSGAEVAKPGASVKLSCKASGYTFTNYWMQWIKQA
PGQGLEWIGAVYPGDGDTRHTQKFKGKATLTADKSTSTAY
MEVSSLRSEDTAVYYCARGFTAYGMDYWGQGTTVTVSS.

In some embodiments, the ADGRE2 antigen binding domain comprises a variable heavy chain amino acid sequence having at least about 85%, about 90%, about 91%, about 92%, about 93%, about 94%, about 95%, about 96%, about 97%, about 98%, or about 99% sequence identity to SEQ ID NO: 7, 9, 11, 13, 15, 17, or 30.

In some embodiments, the ADGRE2 antigen binding domain comprises a heavy chain variable region amino acid sequence having at least about 85%, about 90%, about 91%, about 92%, about 93%, about 94%, about 95%, about 96%, about 97%, about 98%, or about 99% sequence identity to SEQ ID NO: 7, 9, 11, 13, 15, 17, or 30 while also including one or more of the VH CDR1, vHCDR2, and/or vHCDR3 sequences described herein.

In some embodiments, the ADGRE2 antigen binding domain comprises a heavy chain variable region amino acid sequence identical to SEQ ID NO: 7, 9, 11, 13, 15, 17 or 30. In certain embodiments, the V H comprises an amino acid sequence that is at least about 80% (e.g., at least about 85%, at least about 90%, or at least about 95%) identical or homologous to the amino acid sequence set forth in SEQ ID NO: 7, 9, 11, 13, 15, 17 or 30. For example, the VH comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical or homologous to the amino acid sequence set forth in SEQ ID NO: 7, 9, 11, 13, 15, 17 or 30. In some embodiments, the anti- ADGRE2 antigen binding domain comprises no more than 30, 29, 28, 27, 26, 25, 24, 23, 22, 21, 20, 19, 18, 17, 16, 15, 14, 13, 12, 11, 10, 9, 8, 7, 6, 5, 4, 3, or 2 amino acid substitutions relative to SEQ ID NO: 7, 9, 11, 13, 15, 17 or 30.

As will be understood by those of skill in the art, any such heavy chain constant domain sequence may be readily combined, e.g., by techniques of molecular biology, with any other antibody sequences or domains provided herein or otherwise known in the art, including any framework regions, CDRs, or constant domains, or portions thereof as disclosed herein or otherwise known in the art, as may be present in an antibody or an antigen-binding fragment thereof of any format as disclosed herein or otherwise known in the art.

The present invention additionally provides an ADGRE2 antigen binding domain or fragment thereof comprising various specified sequences in one or more light chain variable regions, including in the light chain complementary determining regions LCDR1-3. In various embodiments, molecules with specified light chain variable regions are provided with heavy chain sequences as discussed above. In certain embodiments, the CDRs are identified according to the IMGH numbering system.

Thus, in one aspect, the present invention provides an ADGRE2 antigen binding domain or fragment thereof comprising a light chain variable region with complementarity determining region (CDR) sequences of SSVSY (SEQ ID NO: 4), an LCDR2 comprising an amino acid sequence of DTS (SEQ ID NO: 5), and an LCDR3 comprising an amino acid sequence of QQWSSNPLT (SEQ ID NO: 6).

In some embodiments, the ADGRE2 antigen binding domain or fragment thereof comprises an immunoglobulin light chain variable (VL) region comprising an amino acid sequence that is at least about 90% identical to SEQ ID NO: 8, 10, 12, 14, 16, 18, or 31 and an immunoglobulin heavy chain variable (VH) region comprising an amino acid sequence that is at least about 90% identical to SEQ ID NO: 7, 9, 11, 13, 15, 17 or 30.

In some embodiments, the VL region comprises an amino acid sequence that is at least about 95% identical to SEQ ID NOs: 8, 10, 12, 14, 16, 18, or 31.

In some embodiments, the ADGRE2 antigen binding domain or fragment thereof comprises a light chain variable region (VL) comprising an amino acid sequence of

(SEQ ID NO: 8)
EIVLTQSPATLSLSPGERATLSCSASSSVSYMHWYQQKPG
LAPRLLIYDTSKLASGIPDRFSGSGSGTDFTLTISRLEPE
DFAVYYCQQWSSNPLTFGQGTKVEIK.

In some embodiments, the light chain variable comprises an amino acid sequence of

(SEQ ID NO: 10)
EIVLTQSPATMSASPGERVTMSCSASSSVSYMHWYQQKSG
LSPKRWIYDTSKLASGVPDRFSGSGSGTDYTFTISRMEPE
DFATYYCQQWSSNPLTFGGGTKLEIK.

In some embodiments, the light chain variable comprises an amino acid sequence of

(SEQ ID NO: 12)
EIVLTQSPATMSASPGERVTMSCSASSSVSYMHWYQQKSG
LSPKRWIYDTSKLASGVPDRFSGSGSGTDYTFTISRMEPE
DFATYYCQQWSSNPLTFGGGTKLEIK.

In some embodiments, the light chain variable comprises an amino acid sequence of

(SEQ ID NO: 14)
EIVLTQSPATMSASPGERVTMSCSASSSVSYMHWYQQKSGQSPKRWIYDT
SKLASGVPARFSGSGSGTDYTFTISSMEPEDFATYYCQQWSSNPLTFGGG
TKLEIK.

In some embodiments, the light chain variable comprises an amino acid sequence of

(SEQ ID NO: 16)
QIVLTQSPATLSLSPGERATLTCSASSSVSYMHWYQQKPGLSPKRWIYDT
SKLASGVPDRFSGSGSGTDYTFTIRRLEPEDFATYYCQQWSSNPLTFGQG
TKVEIK.

In some embodiments, the light chain variable comprises an amino acid sequence of

(SEQ ID NO: 18)
EIVLTQSPATLSASPGERVTMSCSASSSVSYMHWYQQKPGLAPRRWIYDT
SKLASGVPDRFSGSGSGTDYTFTISRMEPEDFATYYCQQWSSNPLTFGGG
TKLEIK.

In some embodiments, the light chain variable comprises an amino acid sequence of

(SEQ ID NO: 31)
EIVLTQSPATMSASPGERVTMSCSASSSVSYMHWYQQKSGQSPKRWIYDT
SKLASGVPARFSGSGSGTDYTFTISSMEPEDFATYYCQQWSSNPLTFGGG
TKLEIK.

In some embodiments, the ADGRE2 antigen binding domain comprises a light chain amino acid sequence having at least about 85%, about 90%, about 91%, about 92%, about 93%, about 94%, about 95%, about 96%, about 97%, about 98%, or about 99% sequence identity to SEQ ID NOs: 8, 10, 12, 14, 16, 18 or 31.

In some embodiments, the ADGRE2 antigen binding domain comprises a light chain amino acid sequence having at least about 85%, about 90%, about 95%, about 98%, or about 99% sequence identity to SEQ ID NOs: 8, 10, 12, 14, 16, 18 or 31 while also including one or more of the VL CDR1, vLCDR2, and/or vLCDR3 sequences described herein.

In some embodiments, the ADGRE2 antigen binding domain or fragment thereof comprises a light chain amino acid sequence identical to SEQ ID NOs: 8, 10, 12, 14, 16, 18 or 31. In some embodiments, the ADGRE2 antigen binding domain comprises no more than 30, 29, 28, 27, 26, 25, 24, 23, 22, 21, 20, 19, 18, 17, 16, 15, 14, 13, 12, 11, 10, 9, 8, 7, 6, 5, 4, 3, or 2 amino acid substitutions relative to SEQ ID NOs: 8, 10, 12, 14, 16, 18 or 31.

As will be understood by those of skill in the art, any such light chain CDR sequence may be readily combined, e.g., by techniques of molecular biology, with any other antibody sequences or domains provided herein or otherwise known in the art, including any framework regions, CDRs, or constant domains, or portions thereof as disclosed herein or otherwise known in the art, as may be present in an antibody or an antigen-binding fragment thereof of any format as disclosed herein or otherwise known in the art.

In some embodiments, an ADGRE2 antigen binding domain described herein includes a light chain that includes any light chain constant domain sequence, e.g., a constant sequence of a light chain known to those of skill in the art. As those of skill in the art will be aware, a light chain constant domain may be a kappa light chain constant domain or a lambda light chain constant domain. In certain embodiments, the constant domain of a light chain as disclosed herein is a kappa light chain constant domain. In various embodiments, an ADGRE2 antigen binding domain described herein includes a light chain constant domain.

In one aspect, the present invention provides an ADGRE2 antigen binding domain or fragment thereof comprising a heavy chain variable region with complementarity determining region (CDR) sequences of VH CDR1: GYTFTNYW (SEQ ID NO: 1), VH CDR2: VYPGDGDT (SEQ ID NO: 2) and VH CDR3: ARGFTAYGMDY (SEQ ID NO: 3), and light chain variable region with complementarity determining region (CDR) sequences of SSVSY (SEQ ID NO: 4), an LCDR2 comprising an amino acid sequence of DTS (SEQ ID NO: 5), and an LCDR3 comprising an amino acid sequence of QQWSSNPLT (SEQ ID NO: 6).

The table below shows exemplary heavy chain variable region (VH) and light chain variable region (VL) sequences of humanized ADGRE2 antibodies disclosed herein.

| | Anti-ADGRE2 Variable Heavy and |
|---|---|
| Domain | Light Chain Sequences |
| VH_1 | QVQLQQSGAEVAKPGASVKLSCKASGYTFTNYWMQWIKQAP |
| | GQGLEWIGAVYPGDGDTRHTQKFKGKATLTADKSTSTAYME |
| | VSSLRSEDTAVYYCARGFTAYGMDYWGQGTTVTVSS (SEQ |
| | ID NO: 7) |
| VL_1 | EIVLTQSPATMSASPGERVTMSCSASSSVSYMHWYQQKSGQ |
| | SPKRWIYDTSKLASGVPARFSGSGSGTDYTFTISSMEPEDF |
| | ATYYCQQWSSNPLTFGGGTKLEIK (SEQ ID NO: 14) |
| VH_2 | QVQLQQSGAEVKKPGASVKLSCKASGYTFTNYWMQWIRQAP |
| | GQGLEWIGAVYPGDGDTRYTQKFQGRATLTADTSISTAYME |
| | VSRLRSDDTAVYYCARGFTAYGMDYWGQGTTVTVSS (SEQ |
| | ID NO: 9) |
| VL_2 | EIVLTQSPATMSASPGERVTMSCSASSSVSYMHWYQQKSGL |
| | SPKRWIYDTSKLASGVPDRFSGSGSGTDYTFTISRMEPEDF |
| | ATYYCQQWSSNPLTFGGGTKLEIK (SEQ ID NO: 10) |
| VH_3 | QVQLQQSGAEVKKPGASVKLSCKASGYTFTNYWMQWVRQAP |
| | GQGLEWIGAVYPGDGDTRYTQKFQGRATLTADTSTSTVYME |
| | VSSLRSEDTAVYYCARGFTAYGMDYWGQGTTVTVSS (SEQ |
| | ID NO: 11) |
| VL_3 | EIVLTQSPATMSASPGERVTMSCSASSSVSYMHWYQQKSGL |
| | SPKRWIYDTSKLASGVPDRFSGSGSGTDYTFTISRMEPEDF |
| | ATYYCQQWSSNPLTFGGGTKLEIK (SEQ ID NO: 12) |
| VH_4 | QVQLQQSGAEVKKPGASVKVSCKASGYTFTNYWMQWVRQAP |
| | GQGLEWMGAVYPGDGDTRHTQKFKGRVTMTRDTSTSTVYME |
| | LSSLRSEDTAVYYCARGFTAYGMDYWGQGTLVTVSS (SEQ |
| | ID NO: 13) |
| VL_4 | EIVLTQSPATLSLSPGERATLSCSASSSVSYMHWYQQKPGL |
| | APRLLIYDTSKLASGIPDRFSGSGSGTDFTLTISRLEPEDF |
| | AVYYCQQWSSNPLTFGQGTKVEIK (SEQ ID NO: 8) |
| VH_5 | QVQLQQSGAEVKKPGASVKVSCKASGYTFTNYWMQWVRQAP |
| | GQGLEWIGAVYPGDGDTRHTQKFKGRVTMTADKSTSTVYME |
| | LSSLRSEDTAVYYCARGFTAYGMDYWGQGTLVTVSSAST |
| | (SEQ ID NO: 15) |
| VL_5 | QIVLTQSPATLSLSPGERATLTCSASSSVSYMHWYQQKPGL |
| | SPKRWIYDTSKLASGVPDRESGSGSGTDYTFTIRRLEPEDF |
| | ATYYCQQWSSNPLTFGQGTKVEIK (SEQ ID NO: 16) |
| VH_6 | QVQLQQSGAEVKKPGASVKLSCKASGYTFTNYWMQWIRQAP |
| | GQGLEWIGAVYPGDGDTRYTQKFQGRATLTADTSTSTAYME |
| | VSSLRSEDTAVYYCARGFTAYGMDYWGQGTTVTVSS (SEQ |
| | ID NO: 17) |
| VL_6 | EIVLTQSPATLSASPGERVTMSCSASSSVSYMHWYQQKPGL |
| | APRRWIYDTSKLASGVPDRESGSGSGTDYTFTISRMEPEDF |
| | ATYYCQQWSSNPLTFGGGTKLEIK (SEQ ID NO: 18). |
| VH_7 | QVQLVQSGAEVAKPGASVKLSCKASGYTFTNYWMQWIKQAP |
| | GQGLEWIGAVYPGDGDTRHTQKFKGKATLTADKSTSTAYME |
| | VSSLRSEDTAVYYCARGFTAYGMDYWGQGTTVTVSS (SEQ |
| | ID NO: 30) |
| VL_7 | EIVLTQSPATMSASPGERVTMSCSASSSVSYMHWYQQKSGQ |
| | SPKRWIYDTSKLASGVPARFSGSGSGTDYTFTISSMEPEDF |
| | ATYYCQQWSSNPLTFGGGTKLEIK (SEQ ID NO: 31). |

In various embodiments, an ADGRE2 antigen binding domain disclosed herein is a homodimeric monoclonal antibody. In various embodiments, an ADGRE2 antigen binding domain disclosed herein is a heterodimeric antibody. In various embodiments, an ADGRE2 antigen binding domain is, e.g., a typical antibody or a diabody, triabody, tetrabody, minibody, maxibody, tandab, DVD, BiTe, scFv, TandAb scFv, Fab, Fab₂, Fab₃, F(ab′)₂, or the like, or any combination thereof.

In some embodiments, the disclosure provides fusion proteins comprising one or more variable domains or engineered antibodies as described herein, or portion thereof, and one or more additional polypeptides.

### Exemplary Single Chain Variable Fragments

In some embodiments, the ADGRE2 binder according to the present invention may be a scFv that specifically binds to ADGRE2. A “Single-chain Fv” or “scFv” comprises the VH and VL domains of antibody, wherein these domains are present in a single polypeptide chain and in either orientation (e.g., VL-VH or VH-VL). Typically, scFv can be in a form of VH-linker-VL or VL-linker-VH.

The linker to link the VH and VL chain may comprise an amino acid sequence of (GGGGS)n (n is an integer of at least 1, 2, 3, 4, 5, 6, 7, 8, 9, or 10). In some embodiments, the linker comprises an amino acid sequence at least about 70%, at least about 75%, at least 85%, at least about 85%, at least about 90%, at least about 91%, at least about 92%, at least about 93%, at least about 94%, at least about 95%, at least 96%, at least about 97%, at least about 98%, at least about 99%, or 100% identical to one of SEQ ID NOs: 24-27.

In some embodiments, the anti-ADGRE2 scFv comprises a linker comprising amino acid sequence GGGGSGGGGSGGGGS (SEQ ID NO: 24).

In some embodiments, the linker comprises GGGGSGGGGSGGGSGGGGS (SEQ ID NO: 25).

In some embodiments, the linker comprises

| (SEQ ID NO: 26) | |
|---|---|
| | GGGGSGGGGSGGGGSGGGSGGGGS |

In some embodiments, the linker comprises

| (SEQ ID NO: 27) | |
|---|---|
| | GGGGSGGGGSGGGGSGGGGSGGGSGGGGS. |

In some embodiments, the disclosure provides a single-chain variable fragment. In some embodiments, the scFv is a human scFv. A “single-chain variable fragment” or “scFv” refers to a fusion protein of the variable regions of the heavy (V_{H}) and light chains (V_{L}) of an immunoglobulin (e.g., mouse or human) covalently linked to form a V_{H}::VL heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the VH with the N-terminus of the V_{L}. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. In certain embodiments, the linker is a G4S linker (GGGGS-SEQ ID NO: 45).

Alternatively or additionally, the scFv may be derived from Fab's (instead of from an antibody, e.g., obtained from Fab libraries). In certain embodiments, the ADGRE2 antigen binding domain or fragment thereof is a Fab. In certain embodiments, the Fab is crosslinked. In certain embodiments, the ADGRE2 antigen binding domain or fragment thereof is a F(ab)₂. Any of the foregoing molecules may be comprised in a fusion protein with a heterologous sequence to form an anti-ADGRE2 antigen antibody or an antigen-binding fragment thereof.

In certain embodiments, the ADGRE2 antigen binding domain or fragment thereof binds to ADGRE2 (e.g., human ADGRE2) with a dissociation constant (K_{D}) of at least about 1×10⁻⁶ M, at least about 1×10⁻⁷ M, at least about 1×10⁻⁸ M, at least about 1×10⁻⁹ M, or at least about 1×10⁻¹⁰ M. In certain embodiments, the ADGRE2 antigen binding domain or fragment thereof binds to ADGRE2 (e.g., human ADGRE2) with a dissociation constant (K_{D}) of at least about 2×10⁻⁸ M. In certain embodiments, the ADGRE2 antigen binding domain or fragment thereof binds to ADGRE2 (e.g., human ADGRE2) with a dissociation constant (K_{D}) of between about 2×10⁻⁸ M and about 8×10⁻⁹ M.

In some embodiments, the ADGRE2 antigen binding domain or fragment thereof binds to ADGRE2 (e.g., human ADGRE2) with a dissociation constant (K_{D}) between about 1 nM and 50 nM, about 5 nM and 30 nM, about 5 nM and 25 nM, or about 8 nM and 20 nM. In some embodiments, the ADGRE2 antigen binding domain or fragment thereof binds to ADGRE2 (e.g., human ADGRE2) with a dissociation constant (K_{D}) of at least about 50 nM, at least about 40 nM, at least about 35 nM, at least about 30 nM, at least about 25 nM, at least about 20 nM, at least about 19 nM, at least about 18 nM, at least about 17 nM, at least about 16 nM, at least about 15 nM, at least about 14 nM, at least about 13 nM, at least about 12 nM, at least about 11 nM, at least about 10 nM, at least about 9 nM, at least about 8 nM, at least about 7 nM, at least about 6 nM, at least about 5 nM.

In some embodiments, the anti-ADGRE2 scFv comprises a heavy chain variable region and/or a light chain variable region comprising the amino acid sequence set forth in SEQ ID NO: 19, SEQ ID NO: 20, SEQ ID NO: 21, SEQ ID NO: 22 or SEQ ID NO: 68.

In some embodiments, the ADGRE2 antigen binding domain or fragment thereof comprises a conservative sequence modification (e.g., ADGRE2 antigen binding domain or fragment thereof described herein). In some embodiments, the conservative sequence modification is an amino acid modification that does not significantly affect or alter the binding characteristics of the presently disclosed ADGRE2 antigen binding domain or fragment thereof (e.g., the antibody or fragment thereof) comprising the amino acid sequence. Conservative modifications can include amino acid substitutions, additions and deletions. Modifications can be introduced into the anti-ADGRE2 antibodies or fragments thereof by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function. In certain embodiments, no more than one, no more than two, no more than three, no more than four, no more than five residues within a specified sequence or a CDR region are altered.

In some embodiments, the light chain and/or heavy chain of the anti-ADGRE2 scFv comprise a signal peptide or signal sequence. The terms signal peptide and signal sequence are used interchangeably herein. In some embodiments, the signal peptide comprises an amino acid sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% homology or identity to the amino acid sequence

| (SEQ ID NO: 50) | |
|---|---|
| | MDWTWRILFLVAAATGAHS, |
| (SEQ ID NO: 51) | |
| | MALPVTALLLPLALLLHA, |
| (SEQ ID NO: 33) | |
| | METDTLLLWVLLLWVPGSTG, |
| (SEQ ID NO: 34) | |
| | MYRMQLLSCIALSLALVTNS, |
| (SEQ ID NO: 35) | |
| | METPAQLLFLLLLWLPDTTG, |
| (SEQ ID NO: 36) | |
| | MALPVTALLLPLALLLHAARP, |
| (SEQ ID NO: 37) | |
| | MKWVTFISLLESSAYS, |
| (SEQ ID NO: 38) | |
| | MDSKGSSQKGSRLLLLLVVSNLLLCQGVVS, |
| (SEQ ID NO: 28) | |
| | MDMRVPAQLLGLLLLWLPDTRC, |
| | or |
| (SEQ ID NO: 29) | |
| | MEFGLSWVFLVALLRGVQC. |

In some embodiments, the signal peptide comprises MDWTWRILFLVAAATGAHS (SEQ ID NO: 50). In some embodiments, the signal peptide comprises MALPVTALLLPLALLLHA (SEQ ID NO: 51). In some embodiments, the signal peptide comprises METDTLLLWVLLLWVPGSTG (SEQ ID NO: 33). In some embodiments, the signal peptide comprises MYRMQLLSCIALSLALVTNS (SEQ ID NO: 34). In some embodiments, the signal peptide comprises METPAQLLFLLLLWLPDTTG (SEQ ID NO: 35). In some embodiments, the signal peptide comprises MALPVTALLLPLALLLHAARP (SEQ ID NO: 36). In some embodiments, the signal peptide comprises MKWVTFISLLFSSAYS (SEQ ID NO: 37). In some embodiments, the signal peptide comprises MDSKGSSQKGSRLLLLLVVSNLLLCQGVVS (SEQ ID NO: 38). In some embodiments, the signal peptide comprises MDMRVPAQLLGLLLLWLPDTRC (SEQ ID NO: 28). In some embodiments, the signal peptide comprises MEFGLSWVFLVALLRGVQC (SEQ ID NO: 29).

In some embodiments, the anti-ADGRE2 scFv comprises an amino acid sequence of

(scFv “K”)
(SEQ ID NO: 19)
QVQLQQSGAEVAKPGASVKLSCKASGYTFTNYWMQWIKQAPGQGLEWIGA
VYPGDGDTRHTQKFKGKATLTADKSTSTAYMEVSSLRSEDTAVYYCARGF
TAYGMDYWGQGTTVTVSSGGGGSGGGGSGGGGSEIVLTQSPATMSASPGE
RVTMSCSASSSVSYMHWYQQKSGQSPKRWIYDTSKLASGVPARFSGSGSG
TDYTFTISSMEPEDFATYYCQQWSSNPLTFGGGTKLEIK;
(scFv “B”)
(SEQ ID NO: 20)
QVQLQQSGAEVKKPGASVKLSCKASGYTFTNYWMQWIRQAPGQGLEWIGA
VYPGDGDTRYTQKFQGRATLTADTSISTAYMEVSRLRSDDTAVYYCARGF
TAYGMDYWGQGTTVTVSSGGGGSGGGGSGGGGSEIVLTQSPATMSASPGE
RVTMSCSASSSVSYMHWYQQKSGLSPKRWIYDTSKLASGVPDRESGSGSG
TDYTFTISRMEPEDFATYYCQQWSSNPLTFGGGTKLEIK;
(scFv “N”)
(SEQ ID NO: 21)
QVQLQQSGAEVKKPGASVKLSCKASGYTFTNYWMQWVRQAPGQGLEWIGA
VYPGDGDTRYTQKFQGRATLTADTSTSTVYMEVSSLRSEDTAVYYCARGF
TAYGMDYWGQGTTVTVSSGGGGSGGGGSGGGGSEIVLTQSPATMSASPGE
RVTMSCSASSSVSYMHWYQQKSGLSPKRWIYDTSKLASGVPDRFSGSGSG
TDYTFTISRMEPEDFATYYCQQWSSNPLTFGGGTKLEIK;
or
(scFv “A”)
(SEQ ID NO: 22)
QVQLQQSGAEVKKPGASVKVSCKASGYTFTNYWMQWVRQAPGQGLEWMGA
VYPGDGDTRHTQKFKGRVTMTRDTSTSTVYMELSSLRSEDTAVYYCARGF
TAYGMDYWGQGTLVTVSSASTGGGGSGGGGSGGGGSEIVLTQSPATLSLS
PGERATLSCSASSSVSYMHWYQQKPGLAPRLLIYDTSKLASGIPDRESGS
GSGTDFTLTISRLEPEDFAVYYCQQWSSNPLTFGQGTKVEIK.
(SEQ ID NO: 68)
QVQLQQSGAEVKKPGASVKVSCKASGYTFTNYWMQWVRQAPGQGLEWIGA
VYPGDGDTRHTQKFKGRVTMTADKSTSTVYMELSSLRSEDTAVYYCARGF
TAYGMDYWGQGTLVTVSSASTGGGGSGGGGSGGGGSQIVLTQSPATLSLS
PGERATLTCSASSSVSYMHWYQQKPGLSPKRWIYDTSKLASGVPDRFSGS
GSGTDYTFTIRRLEPEDFATYYCQQWSSNPLTFGQGTKVEIK

In some embodiments, anti-ADGRE2 scFv comprises an amino acid sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% homology or identity to SEQ ID NO: 19, 20, 21 22 or 68.

In some embodiments, the anti-ADGRE2 scFv comprises the amino acid sequence set forth in SEQ ID NO: 19, 20, 21, 22 or 68. In some embodiments, the anti-ADGRE2 scFv comprises the amino acid sequence set forth in SEQ ID NO: 19. In some embodiments, the anti-ADGRE2 scFv comprises the amino acid sequence set forth in SEQ ID NO: 20. In some embodiments, the anti-ADGRE2 scFv comprises the amino acid sequence set forth in SEQ ID NO: 21. In some embodiments, the anti-ADGRE2 scFv comprises the amino acid sequence set forth in SEQ ID NO: 22. In some embodiments, the anti-ADGRE2 scFv comprises the amino acid sequence set forth in SEQ ID NO: 68.

### Measuring Interactions of Binding Moieties and Targets

The binding properties of an antibody or fragment thereof described herein (e.g., an ADGRE2 antigen binding domain described herein) to a target (e.g., ADGRE2) can be measured by methods known in the art, e.g., one of the following methods: BIACORE analysis, Enzyme Linked Immunosorbent Assay (ELISA), x-ray crystallography, sequence analysis and scanning mutagenesis. The binding interaction of an antibody and ADGRE2 and/or FcRn can be analyzed using surface plasmon resonance (SPR). SPR or Biomolecular Interaction Analysis (BIA) detects bio-specific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Cum Opin. Struct. Biol. 5:699-705 and on-line resources provide by BIAcore International AB (Uppsala, Sweden). Additionally, a KinExA® (Kinetic Exclusion Assay) assay, available from Sapidyne Instruments (Boise, Id.) can also be used.

Information from SPR can be used to provide an accurate and quantitative measure of the equilibrium dissociation constant (K_{D}), and kinetic parameters, including Kₒₙ and K_{off}, for the binding of a binding moiety to a target (e.g., an ADGRE2 antigen binding domain to ADGRE2). Such data can be used to compare different molecules. Information from SPR can also be used to develop structure-activity relationships (SAR). For example, the kinetic and equilibrium binding parameters of particular binding moieties to targets at various pH levels can be evaluated. Variant amino acids at given positions can be identified that correlate with particular binding parameters, e.g., high affinity, low affinity, and slow K_{off}, at particular pH levels.

In some embodiments, provided herein is a chimeric receptor that binds to human ADGRE2 with higher affinity than to another species of ADGRE2. In certain embodiments, provided herein is an antibody or fragment thereof that binds to human ADGRE2 with a 5%, 10%, 15%, 20%, 25%, 30%, 35%, 40%, 45%, 50%, 55%, 60%, 65%, 70%, 75%, 80%, 85%, 90%, 95% or higher affinity than to another species of ADGRE2 as measured by, e.g., a radioimmunoassay, surface plasmon resonance, or kinetic exclusion assay. In a specific embodiment, a chimeric antigen receptor comprising a ADGRE2 binder, which binds to human ADGRE2, will bind to another species of ADGRE2 protein with less than 10%, 15%, or 20% of the binding of the antibody or fragment thereof to the human ADGRE2 protein as measured by, e.g., a radioimmunoassay, surface plasmon resonance, or kinetic exclusion assay.

### Hinge Region

In certain aspects, the chimeric receptor (e.g., CAR or CCR) polypeptide of the present invention can include a hinge domain positioned between the extracellular antigen binding domain and the transmembrane domain. A hinge domain may be included in chimeric receptor polypeptides to provide adequate distance between the antigen binding domain and the cell surface or to alleviate possible steric hindrance that could adversely affect antigen binding or effector function of chimeric receptor -gene modified CB-NK cells. For example, the hinge domain can position the antigen binding domain away from the effector cell surface to enable proper cell/cell contact, antigen binding and activation.

In some embodiments, the hinge domain is of a particular length, such as 10-50, 10-40, 10-30, 10-20, 10-15, 20-50, 20-40, 20-30, 15-50, 15-45, 15-30, 15-20, 12-20, 12-15, or 15-20 amino acids in length.

In some embodiments, the hinge domain is derived from human CD28 hinge domain. As a non-limiting example, the hinge domain of the present chimeric receptor polypeptide comprises a CD28 hinge domain having the amino acid sequence of SEQ ID NO: 53: IEVMYPPPYLDNEKSNGTIIHVKGKHLCPSPLFPGPSKP. In certain specific aspects, the hinge domain comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least 98%, or at least about 99% identical to SEQ ID NO: 53.

In some embodiments, the hinge domain is derived from human CD8 hinge domain. As a non-limiting example, the hinge domain of the present chimeric receptor polypeptide comprises a CD8 hinge domain having the amino acid sequence of SEQ ID NO: 69: TTTPAPRPPTPAPTIASQPLSLRPEACRPAAGGAVHTRGLDFACD. In some aspects, the hinge domain comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least 98%, or at least about 99% identical to SEQ ID NO: 69.

Alternatively, and optionally, one or more hinge or spacer domains derived from other protein may be used. As used herein, the term “spacer domain” refers to the region that moves the antigen binding domain away from the effector cell surface to enable proper cell/cell contact, antigen binding and activation (Patel et al., Gene Therapy, 1999; 6: 412-419). The spacer domain may be derived either from a natural, synthetic, semi-synthetic, or recombinant source. For example, the hinge and spacer domains can be derived from a human IgG hinge domain, a CD8a hinge domain, or an Fc domain from a human immunoglobulin (e.g., IgG1, IgG2, IgG3, IgG4, IgAl, IgA2, IgM, IgD or IgE) that binds to an Fc receptor.

In some embodiments, the ADGRE2 targeting chimeric receptors contemplated herein may comprise a modified hinge domain and/or spacer domain. The modified hinge domain and/or spacer domain may comprise up to 30% amino acid changes (e.g., up to 25%, 20%, 15%, 10%, or 5% amino acid substitutions or deletions), or a portion of a naturally occurring hinge region that is at least 10 amino acids (e.g, at least 12, 13, 14 or 15 amino acids) in length with up to 30% amino acid changes (e.g., up to 25%, 20%, 15%, 10%, or 5% amino acid substitutions or deletions), or a portion of a naturally occurring hinge region that comprises the core hinge region (which may be 4, 5, 6, 7, 8, 9, 10, 11, 12, 13, 14, or 15, or at least 4, 5, 6, 7, 8, 9, 10, 11, 12, 13, 14, or 15 amino acids in length).

### Transmembrane Domain

In various embodiments, the extracellular antigen binding domain and the intracellular signaling domain of the ADGRE2 targeting chimeric receptors (CAR or CCR) of the present invention may be fused by a transmembrane domain. The transmembrane domain can also anchor the chimeric receptors to the plasma membrane of the CB-NK cell. The transmembrane domain may be derived from any membrane -bound or transmembrane protein. The transmembrane domain may be derived from a natural, synthetic, semi-synthetic, or recombinant source. In some aspects, the amino acid sequence of the transmembrane domain may be modified or substituted so as to minimize, e.g., interactions with the binding domains of the native binding partner present in the same CAR-expressing cell.

In some embodiments, the transmembrane domain of the ADGRE2 targeting CAR or CCR of the present invention is derived from the transmembrane domain of human CD28. For example, the CD28 transmembrane domain of the ADGRE2 targeting CAR polypeptide may comprise the amino acid sequence of SEQ ID NO: 54: FWVLVVVGGVLACYSLLVTVAFIIFWV. In some aspects, for example, the transmembrane domain comprises a sequence at least 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO: 54.

In some embodiments, the transmembrane domain of the ADGRE2 targeting CAR or CCR of the present invention is derived from the transmembrane domain of human CD8. For example, the CD8 transmembrane domain of the ADGRE2 targeting CAR polypeptide may comprise the amino acid sequence of SEQ ID NO: 70: IYIWAPLAGTCGVLLLSLVITLYCNL. In some embodiments, the transmembrane domain comprises a sequence at least 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO: 70.

Alternatively, the transmembrane domain of the CAR may include the transmembrane region(s) of the alpha, beta or zeta chain of the T-cell receptor, CD3 epsilon, CD45, CD4, CDS, CD8, CD9, CD16, CD22, CD33, CD37, CD64, CD80, CD86, CD134, CD137, CD154.

### Cytoplasmic Domains

The cytoplasmic region of a CAR polypeptide includes an intracellular activation signaling domain. The intracellular signaling domain of the chimeric antigen receptor is responsible for activation of at least one of the normal effector functions of the immune cell engineered to express a chimeric antigen receptor. The term “effector function” in general refers to a specialized function of a differentiated cell. Effector function of an immune cell (e.g., CB-NK cell) can be the cytolytic activity, cytotoxic activity or helper activity including the secretion of cytokines, to kill tumor cells. In this context, the term “intracellular signaling domain” refers to the portion of a protein that transduces the effector function signal and directs the cell to perform a specialized function. In some embodiments, the intracellular signaling domain is derived from the intracellular signaling domain of a native activating protein. Examples of such native activating proteins such as native receptors including the zeta chain of the T-cell receptor or any of its homologs (e.g., eta, delta, gamma, or epsilon), MB1 chain, B29, Fc RIII, Fc RI, signaling molecules such as CD3ξ, CD28, CD27, 4-IBB, DAP10, OX40, and other similar molecules. While usually the entire intracellular signaling domain will be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term “intracellular signaling domain” is thus meant to include a truncated portion of the intracellular signaling domain sufficient to transduce the effector function signal, upon CAR binding to a target.

In one preferred embodiment, the human CD3ξ intracellular signaling domain is used as the intracellular signaling domain for a ADGRE2 targeting CAR polypeptide of the present invention. An example of the CD3ξ intracellular signaling domain comprises the amino acid sequence of SEQ ID NO: 55:

RVKFSRSADAPAYQQGQNQLYNELNLGRREEYDVLDKRRGRDPEMGGK PRRKNPQEGLYNELQKDKMAEAYSEIGMKGERRRGKGHDGLYQGLSTATKDTYDALH MQALPPR (SEQ ID NO: 55). In certain specific aspects, the intracellular signaling domain comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least 98%, or at least about 99% identical to SEQ ID NO: 55.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs. In some embodiments, the human CD3ξ intracellular signaling domain comprise ITAM1, ITAM2 and ITAM3 motifs. In some embodiments, the modified CD3ξ polypeptide comprises a native ITAM1 motif, a variant ITAM2 motif comprising two loss-of-function mutations, and a variant ITAM3 motif comprising two loss-of-function mutations.

In some embodiments, the ITAM1 motif comprises SEQ ID NO: 62: QNQLYNELNLGRREEYDVLDKR. In some embodiments, the variant ITAM1 motif comprises SEQ ID NO: 63: QNQLFNELNLGRREEFDVLDKR.

In some embodiments, the ITAM2 motif comprises SEQ ID NO: 64: QEGLYNELQKDKMAEAYSEIGMK. In some embodiments, the variant ITAM2 motif comprises SEQ ID NO: 65: QEGLFNELQKDKMAEAFSEIGMK.

In some embodiments, the ITAM3 motif comprises SEQ ID NO: 66: HDGLYQGLSTATKDTYDALHMQ. In some embodiments, the variant ITAM3 motif comprises SEQ ID NO: 67: HDGLFQGLSTATKDTFDALHMQ.

In some embodiments, the CAR polypeptide may comprise one or more co-stimulatory signaling domains. A costimulatory intracellular signaling domain refers to the intracellular portion of a costimulatory molecule. The term “costimulatory molecule” refers to the cognate binding partner on an immune cell that specifically binds with a costimulatory ligand, thereby mediating a costimulatory response by the immune cell, such as, but not limited to, proliferation.

In some embodiments, the present ADGRE2 targeting CAR or CCR comprises a costimulatory domain derived from DAP10. An example of the DAP10 derived costimulatory domain comprises the amino acid sequence of SEQ ID NO: 56: LCARPRRSPAQEDGKVYINMPGRG. In certain aspects, the DAP10 costimulatory domain comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least 98%, or at least about 99% identical to SEQ ID NO: 56.

In some embodiments, the present ADGRE2 targeting CAR or CCR comprises a costimulatory domain derived from OX40. An example of the OX40 derived costimulatory domain comprises the amino acid sequence of SEQ ID NO: 71: ALYLLRRDQRLPPDAHKPPGGGSFRTPIQEEQADAHSTLAKI. In certain aspects, the OX40 costimulatory domain comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least 98%, or at least about 99% identical to SEQ ID NO: 71.

The costimulatory domain derived from CD28 may be utilized in the present ADGRE2 targeting CAR polypeptide. An example of CD28 co-stimulatory domain comprises the amino acid sequence of SED ID NO: 57:

| (SEQ ID NO: 57) | |
|---|---|
| | RSKRSRLLHSDYMNMTPRRPGPTRKHYQPYAPPRDFAAYRS |

In addition to the DAP10 derived costimulatory domain and the CD3ξ intracellular signaling domain, a ADGRE2 targeting CAR or CCR polypeptide as described herein may comprise one or more costimulatory domains, and/or one or more intracellular activation signaling domains. The costimulatory domains and intracellular activation signaling domains may be derived from, e.g., an MHC class I molecule, TNF receptor proteins, Immunoglobulin-like proteins, cytokine receptors, integrins, signaling lymphocytic activation molecules (SLAM proteins), activating NK cell receptors, BTLA, Toll ligand receptor, OX40, 4-1BB/CD137, CD2, CD7, CD27, CD28, CD30, CD40, programmed death-1 (PD-1), inducible T cell costimulator (ICOS), CDS gamma, CD3 delta, CD3 epsilon, CD247, CD276 (B7-H3), LIGHT (tumor necrosis factor superfamily member 14; TNFSF1.4), NKG2C, 2B4, Ig alpha (CD79a), Fc gamma receptor, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD 19, CD4, CDSalpha, CDSbeta, 11.2 beta, IL2R gamma, IL7R alpha, ITGA4, VLA1, CD49a, ITGA4, IA4, CD49D, ITGA6, VLA-6, CD49f, ITGAD, ITGAE, CD103, ITGAL, LFA-1, ITGAM, ITGAX, ITGB1, CD29, ITGB2, ICOS, CD18, LFA-1, ITGB7, NKG2D, TNFR2, TRANCE RANKL, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), CD69, SLAMF6 (NTB-A, Ly108), BLAME (SLAMF8), SELPLG (CD 162), LTBR, LAT, GADS, SLP-76, PAG/Cbp, CD 19a, a ligand that specifically binds with CD83, or any combination thereof.

### Other Components

In some embodiments, one or more other polypeptides and/or proteins may be incorporated into the ADGRE2 targeting CAR constructs as described herein. The additional proteins and polypeptides may be utilized for any function, e.g., the activity of the CAR polypeptide and/or any cells expressing the CAR.

In some embodiments, the ADGRE2 targeting CAR polypeptide as described herein may further comprise one or more cytokines. The CAR and the other protein(s) may be separated by a cleavable 2A sequence, for example.

In some embodiments, the cytokine IL-15 is incorporated into the ADGRE2 targeting CAR construct. IL-15 is a proinflammatory cytokine, important for the differentiation and proliferation of T-cells, NK/T-cells, and the development of dendritic cells. An example of IL15 comprises the amino acid sequence of SEQ ID NO: 58:

(SEQ ID NO: 58)
MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS

In some embodiments, the IL15 protein incorporated into the ADGRE2 targeting CAR construct comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least 98%, or at least about 99% identical to SEQ ID NO: 58.

The IL-15 peptide is encoded by the nucleic acid sequence of SEQ ID NO: 59:

(SEQ ID NO: 59)
atgcgcattagcaagccccacctgcggagcatcagcatccagtgctacct
gtgcctgctgctgaacagccacttcctgaccgaggccggcatccacgtgt
tcatcctgggctgcttcagcgccggactgcccaagaccgaggccaactgg
gtgaacgtgatcagcgacctgaagaagatcgaggacctgatccagagcat
gcacatcgacgccaccctgtacaccgagagcgacgtgcaccccagctgca
aggtgaccgccatgaagtgctttctgctggaactgcaggtgatcagcctg
gaaagcggcgacgccagcatccacgacaccgtggagaacctgatcatcct
ggccaacaacagcctgagcagcaacggcaacgtgaccgagagcggctgca
aagagtgcgaggaactggaagagaagaacatcaaagagtttctgcagagc
ttcgtgcacatcgtgcagatgttcatcaacaccagc

In some aspects, the CAR polypeptide and other proteins (e.g., IL15) in the same construct are intended to be produced into two different polypeptides, a cleavable 2A sequence may be utilized (e.g., T2A, F2A, and E2A). The 2A self-cleaving peptides (i.e., 2A peptides), are a class of 18-22 AA -long peptides, which can induce ribosomal skipping during translation of a protein in a cell. These peptides share a core sequence motif of DxExNPGP, and are found in a wide range of viral families. They help generating polyproteins by causing the ribosome to fail at making a peptide bond. The members of 2A peptides are named after the virus in which they have been first described. For example, F2A, the first described 2A peptide, is derived from foot-and-mouth disease virus. The name “2A” itself comes from the gene numbering scheme of this virus.

In some embodiments, the cleavable peptide is E2A. As a non-limiting example, the E2A comprises the sequence of GPQCTNYALLKLAGDVESNPGP (SEQ ID NO: 60). In some embodiments, the cleavable peptide is positioned between the CAR polypeptide and IL-15.

The ADGRE2 targeting CAR as described herein may further comprise a signal peptide. The signal peptide may comprise 3-30, 3-20, 3-15, 5-30, 5-20, 5-15, 10-30, 10-20, or 10-15 amino acid residues. An example of a signal peptide comprises the sequence of SEQ ID NO: 61: MEFGLSWLFLVAILKGVQC.

In some embodiments, the ADGRE2 targeting CAR comprises a signal peptide, a ADGRE2 binding domain, a hinge and a transmembrane domain, a DAP10 costimulatory domain and at least one intracellular signaling domain.

In some embodiments, the ADGRE2 targeting CCR comprises a signal peptide, a ADGRE2 binding domain, a hinge and a transmembrane domain, and a OX40 costimulatory domain.

### Linkers

The ADGRE2 targeting CARs or CCR contemplated herein may comprise linker residues between the various domains. In some embodiments, the ADGRE2 targeting CARs contemplated herein, may comprise one, two, three, four, or five or more linkers. In particular embodiments, the length of a linker is about 1 to about 30 amino acids, about 1 to about 25 amino acids, about 5 to about 30 amino acids, about 5 to about 25 amino acids, bout 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acids in length. The ordinarily skilled artisan will recognize that design of a CAR in particular embodiments can include linkers that are all or partially flexible, such that the linker can include a flexible linker as well as one or more portions that confer less flexible structure to provide for a desired CAR structure.

### Exemplary ADGRE2 Targeting Chimeric Receptor Constructs

Exemplary ADGRE2 targeting chimeric receptors disclosed herein are expressed as a polypeptide construct comprising the chimeric receptor and one or more other components. In some embodiments, the present invention provides a nucleic acid molecule encoding, from N-terminal to C-terminal end:

[symbol]

[symbol]

[symbol]

[symbol]

In some embodiments, the nucleic acid molecule encodes, from N-terminal to C-terminal end:

[symbol]

[symbol]

[symbol]

[symbol]

In some embodiments, the nucleic acid molecule encodes, from N-terminal to C-terminal end:

[symbol]

[symbol]

In some embodiments, the nucleic acid molecule encodes, from N-terminal to C-terminal end:

[symbol]

[symbol]

[symbol]

In some embodiments, the nucleic acid molecule encodes an ADGRE2 targeting CAR or CCR polypeptide construct comprising an amino acid sequence selected from SEQ ID NO: 48, 72-82.

| SEQ ID NO: | Sequence |
|---|---|
| 48 | MDWTWRILELVAAATGAHSQVQLQQSGAEVAKPGASVKLSCKASGYTFTN |
| | YWMQWIKQAPGQGLEWIGAVYPGDGDTRHTQKFKGKATLTADKSTSTAYM |
| | EVSSLRSEDTAVYYCARGFTAYGMDYWGQGTTVTVSSGGGGSGGGGSGGG |
| | GSEIVLTQSPATMSASPGERVIMSCSASSSVSYMHWYQQKSGQSPKRWIY |
| | DTSKLASGVPARFSGSGSGTDYTFTISSMEPEDFATYYCQQWSSNPLTFG |
| | GGTKLEIKRAAAIEVMYPPPYLDNEKSNGTIIHVKGKHLCPSPLFPGPSK |
| | PKDPKFWVLVVVGGVLACYSLLVTVAFIIFWVLCARPRRSPAQEDGKVYI |
| | NMPGRGRVKFSRSADAPAYQQGQNQLYNELNLGRREEYDVLDKRRGRDPE |
| | MGGKPRRKNPQEGLYNELQKDKMAEAYSEIGMKGERRRGKGHDGLYQGLS |
| | TATKDTYDALHMQALPPRGPQCTNYALLKLAGDVESNPGPMRISKPHLRS |
| | ISIQCYLCLLLNSHELTEAGIHVFILGCFSAGLPKTEANWVNVISDLKKI |
| | EDLIQSMHIDATLYTESDVHPSCKVTAMKCFLLELQVISLESGDASIHDT |
| | VENLIILANNSLSSNGNVTESGCKECEELEEKNIKEFLQSFVHIVQMFIN |
| | TS |
| 72 | MDWTWRILFLVAAATGAHSQVQLQQSGAEVAKPGASVKLSCKASGYTFTN |
| | YWMQWIKQAPGQGLEWIGAVYPGDGDTRHTQKFKGKAILTADKSTSTAYM |
| | EVSSLRSEDTAVYYCARGFTAYGMDYWGQGTTVTVSSGGGGSGGGGSGGG |
| | GSEIVLTQSPATMSASPGERVTMSCSASSSVSYMHWYQQKSGQSPKRWIY |
| | DTSKLASGVPARFSGSGSGTDYTFTISSMEPEDFATYYCQQWSSNPLTFG |
| | GGTKLEIKRAAAPTTTPAPRPPTPAPTIASQPLSLRPEACRPAAGGAVHT |
| | RGLDFACDIYIWAPLAGTCGVLLLSLVITLYCNLCARPRRSPAQEDGKVY |
| | INMPGRGRVKFSRSADAPAYQQGQNQLYNELNLGRREEYDVLDKRRGRDP |
| | EMGGKPRRKNPQEGLYNELQKDKMAEAYSEIGMKGERRRGKGHDGLYQGL |
| | STATKDTYDALHMQALPPRGPQCINYALLKLAGDVESNPGPMRISKPHLR |
| | SISIQCYLCLLLNSHELTEAGIHVFILGCFSAGLPKTEANWVNVISDLKK |
| | IEDLIQSMHIDATLYTESDVHPSCKVTAMKCFLLELQVISLESGDASIHD |
| | TVENLIILANNSLSSNGNVTESGCKECEELEEKNIKEFLQSFVHIVQMFI |
| | NTS |
| 73 | MDWTWRILFLVAAATGAHSQVQLQQSGAEVAKPGASVKLSCKASGYTFIN |
| | YWMQWIKQAPGQGLEWIGAVYPGDGDTRHTQKFKGKATLTADKSTSTAYM |
| | EVSSLRSEDTAVYYCARGFTAYGMDYWGQGTTVTVSSGGGGSGGGGSGGG |
| | GSEIVLTQSPATMSASPGERVTMSCSASSSVSYMHWYQQKSGQSPKRWIY |
| | DTSKLASGVPARFSGSGSGTDYTFTISSMEPEDFATYYCQQWSSNPLTFG |
| | GGTKLEIKRAAAPTTTPAPRPPTPAPTIASQPLSLRPEACRPAAGGAVHT |
| | RGLDFACDIYIWAPLAGTCGVLLLSLVITLYCNRDQRLPPDAHKPPGGGS |
| | FRTPIQEEQADAHSTLAKIGSGEGRGSLLTCGDVEENPGPMYRMQLLSCI |
| | ALSLALVINSATSNWVNVISDLKKIEDLIQSMHIDATLYTESDVHPSCKV |
| | TAMKCFLLELQVISLESGDASIHDTVENLIILANNSLSSNGNVTESGCKE |
| | CEELEEKNIKEFLQSFVHIVQMFINTS |
| 74 | MDWTWRILFLVAAATGAHSQVQLQQSGAEVAKPGASVKLSCKASGYTFTN |
| | YWMQWIKQAPGQGLEWIGAVYPGDGDTRHTQKFKGKATLTADKSTSTAYM |
| | EVSSLRSEDTAVYYCARGFTAYGMDYWGQGTTVTVSSGGGGSGGGGSGGG |
| | GSEIVLTQSPATMSASPGERVTMSCSASSSVSYMHWYQQKSGQSPKRWIY |
| | DTSKLASGVPARFSGSGSGTDYTFTISSMEPEDFATYYCQQWSSNPLTFG |
| | GGTKLEIKRAAAIEVMYPPPYLDNEKSNGTIIHVKGKHLCPSPLFPGPSK |
| | PKDPKFWVLVVVGGVLACYSLLVTVAFIIFWVALYLLRRDQRLPPDAHKP |
| | PGGGSFRTPIQEEQADAHSTLAKIGPQCTNYALLKLAGDVESNPGPMRIS |
| | KPHLRSISIQCYLCLLLNSHELTEAGIHVFILGCFSAGLPKTEANWVNVI |
| | SDLKKIEDLIQSMHIDATLYTESDVHPSCKVTAMKCFLLELQVISLESGD |
| | ASIHDTVENLIILANNSLSSNGNVTESGCKECEELEEKNIKEFLQSFVHI |
| | VQMFINTS |
| 75 | MDWTWRILFLVAAATGAHSQVQLQQSGAEVKKPGASVKLSCKASGYTFTN |
| | YWMQWIRQAPGQGLEWIGAVYPGDGDTRYTQKFQGRATLTADTSISTAYM |
| | EVSRLRSDDTAVYYCARGFTAYGMDYWGQGTTVTVSSGGGGSGGGGSGGG |
| | GSEIVLIQSPATMSASPGERVTMSCSASSSVSYMHWYQQKSGLSPKRWIY |
| | DTSKLASGVPDRFSGSGSGTDYTFTISRMEPEDFATYYCQQWSSNPLTFG |
| | GGTKLEIKRAAAIEVMYPPPYLDNEKSNGTIIHVKGKHLCPSPLFPGPSK |
| | PKDPKFWVLVVVGGVLACYSLLVTVAFIIFWVLCARPRRSPAQEDGKVYI |
| | NMPGRGRVKFSRSADAPAYQQGQNQLYNELNLGRREEYDVLDKRRGRDPE |
| | MGGKPRRKNPQEGLYNELQKDKMAEAYSEIGMKGERRRGKGHDGLYQGLS |
| | TATKDTYDALHMQALPPRGPQCTNYALLKLAGDVESNPGPMRISKPHLRS |
| | ISIQCYLCLLLNSHELTEAGIHVFILGCFSAGLPKTEANWVNVISDLKKI |
| | EDLIQSMHIDATLYTESDVHPSCKVTAMKCFLLELQVISLESGDASIHDT |
| | VENLIILANNSLSSNGNVTESGCKECEELEEKNIKEFLQSFVHIVQMFIN |
| | TS |
| 76 | MDWTWRILFLVAAATGAHSQVQLQQSGAEVKKPGASVKLSCKASGYTFTN |
| | YWMQWIRQAPGQGLEWIGAVYPGDGDTRYTQKFQGRATLTADTSISTAYM |
| | EVSRLRSDDTAVYYCARGFTAYGMDYWGQGTTVTVSSGGGGSGGGGSGGG |
| | GSEIVLTQSPATMSASPGERVIMSCSASSSVSYMHWYQQKSGLSPKRWIY |
| | DTSKLASGVPDRFSGSGSGTDYTFTISRMEPEDFATYYCQQWSSNPLTFG |
| | GGTKLEIKRAAAPTTTPAPRPPTPAPTIASQPLSLRPEACRPAAGGAVHT |
| | RGLDFACDIYIWAPLAGTCGVLLLSLVITLYCNLCARPRRSPAQEDGKVY |
| | INMPGRGRVKFSRSADAPAYQQGQNQLYNELNLGRREEYDVLDKRRGRDP |
| | EMGGKPRRKNPQEGLYNELQKDKMAEAYSEIGMKGERRRGKGHDGLYQGL |
| | STATKDTYDALHMQALPPRGPQCTNYALLKLAGDVESNPGPMRISKPHLR |
| | SISIQCYLCLLLNSHFLTEAGIHVFILGCFSAGLPKTEANWVNVISDLKK |
| | IEDLIQSMHIDATLYTESDVHPSCKVTAMKCFLLELQVISLESGDASIHD |
| | TVENLIILANNSLSSNGNVTESGCKECEELEEKNIKEFLQSFVHIVQMFI |
| | NTS |
| 77 | MDWTWRILFLVAAATGAHSQVQLQQSGAEVKKPGASVKLSCKASGYTFTN |
| | YWMQWIRQAPGQGLEWIGAVYPGDGDTRYTQKFQGRATLTADTSISTAYM |
| | EVSRLRSDDTAVYYCARGFTAYGMDYWGQGTTVTVSSGGGGSGGGGSGGG |
| | GSEIVLTQSPATMSASPGERVIMSCSASSSVSYMHWYQQKSGLSPKRWIY |
| | DTSKLASGVPDRESGSGSGTDYTFTISRMEPEDFATYYCQQWSSNPLTFG |
| | GGTKLEIKRAAAPTTTPAPRPPTPAPTIASQPLSLRPEACRPAAGGAVHT |
| | RGLDFACDIYIWAPLAGTCGVLLLSLVITLYCNRDQRLPPDAHKPPGGGS |
| | FRTPIQEEQADAHSTLAKIGSGEGRGSLLTCGDVEENPGPMYRMQLLSCI |
| | ALSLALVINSATSNWVNVISDLKKIEDLIQSMHIDATLYTESDVHPSCKV |
| | TAMKCFLLELQVISLESGDASIHDTVENLIILANNSLSSNGNVTESGCKE |
| | CEELEEKNIKEFLQSFVHIVQMFINTS |
| 78 | MDWTWRILFLVAAATGAHSQVQLQQSGAEVKKPGASVKLSCKASGYTFTN |
| | YWMQWIRQAPGQGLEWIGAVYPGDGDTRYTQKFQGRATLTADTSISTAYM |
| | EVSRLRSDDTAVYYCARGFTAYGMDYWGQGTTVTVSSGGGGSGGGGSGGG |
| | GSEIVLTQSPATMSASPGERVIMSCSASSSVSYMHWYQQKSGLSPKRWIY |
| | DTSKLASGVPDRFSGSGSGTDYTFTISRMEPEDFATYYCQQWSSNPLTFG |
| | GGTKLEIKRAAAIEVMYPPPYLDNEKSNGTIIHVKGKHLCPSPLFPGPSK |
| | PKDPKFWVLVVVGGVLACYSLLVTVAFIIFWVALYLLRRDQRLPPDAHKP |
| | PGGGSFRTPIQEEQADAHSTLAKIGPQCTNYALLKLAGDVESNPGPMRIS |
| | KPHLRSISIQCYLCLLLNSHELTEAGIHVFILGCFSAGLPKTEANWVNVI |
| | SDLKKIEDLIQSMHIDATLYTESDVHPSCKVTAMKCFLLELQVISLESGD |
| | ASIHDTVENLIILANNSLSSNGNVTESGCKECEELEEKNIKEFLQSFVHI |
| | VQMFINTS |
| 79 | MDWTWRILFLVAAATGAHSQVQLQQSGAEVKKPGASVKVSCKASGYTFIN |
| | YWMQWVRQAPGQGLEWIGAVYPGDGDTRHTQKFKGRVTMTADKSTSTVYM |
| | ELSSLRSEDTAVYYCARGFTAYGMDYWGQGTLVTVSSASTGGGGSGGGGS |
| | GGGGSQIVLTQSPATLSLSPGERATLTCSASSSVSYMHWYQQKPGLSPKR |
| | WIYDTSKLASGVPDRFSGSGSGTDYTFTIRRLEPEDFATYYCQQWSSNPL |
| | TFGQGTKVEIKRAAAIEVMYPPPYLDNEKSNGTIIHVKGKHLCPSPLFPG |
| | PSKPKDPKFWVLVVVGGVLACYSLLVTVAFIIFWVLCARPRRSPAQEDGK |
| | VYINMPGRGRVKFSRSADAPAYQQGQNQLYNELNLGRREEYDVLDKRRGR |
| | DPEMGGKPRRKNPQEGLYNELQKDKMAEAYSEIGMKGERRRGKGHDGLYQ |
| | GLSTATKDTYDALHMQALPPRGPQCTNYALLKLAGDVESNPGPMRISKPH |
| | LRSISIQCYLCLLLNSHELTEAGIHVFILGCFSAGLPKTEANWVNVISDL |
| | KKIEDLIQSMHIDATLYTESDVHPSCKVTAMKCFLLELQVISLESGDASI |
| | HDTVENLIILANNSLSSNGNVTESGCKECEELEEKNIKEFLQSFVHIVQM |
| | FINTS |
| 80 | MDWTWRILFLVAAATGAHSQVQLQQSGAEVKKPGASVKVSCKASGYTFTN |
| | YWMQWVRQAPGQGLEWIGAVYPGDGDTRHTQKFKGRVTMTADKSTSTVYM |
| | ELSSLRSEDTAVYYCARGFTAYGMDYWGQGTLVTVSSASTGGGGSGGGGS |
| | GGGGSQIVLTQSPATLSLSPGERATLTCSASSSVSYMHWYQQKPGLSPKR |
| | WIYDTSKLASGVPDRESGSGSGTDYTFTIRRLEPEDFATYYCQQWSSNPL |
| | TFGQGTKVEIKRAAAPTITPAPRPPTPAPTIASQPLSLRPEACRPAAGGA |
| | VHTRGLDFACDIYIWAPLAGTCGVLLLSLVITLYCNLCARPRRSPAQEDG |
| | KVYINMPGRGRVKFSRSADAPAYQQGQNQLYNELNLGRREEYDVLDKRRG |
| | RDPEMGGKPRRKNPQEGLYNELQKDKMAEAYSEIGMKGERRRGKGHDGLY |
| | QGLSTATKDTYDALHMQALPPRGPQCINYALLKLAGDVESNPGPMRISKP |
| | HLRSISIQCYLCLLLNSHELTEAGIHVFILGCFSAGLPKTEANWVNVISD |
| | LKKIEDLIQSMHIDATLYTESDVHPSCKVTAMKCFLLELQVISLESGDAS |
| | IHDTVENLIILANNSLSSNGNVTESGCKECEELEEKNIKEFLQSFVHIVQ |
| | MFINTS |
| 81 | MDWTWRILFLVAAATGAHSQVQLQQSGAEVKKPGASVKVSCKASGYTFTN |
| | YWMQWVRQAPGQGLEWIGAVYPGDGDTRHTQKFKGRVTMTADKSTSTVYM |
| | ELSSLRSEDTAVYYCARGFTAYGMDYWGQGTLVTVSSASTGGGGSGGGGS |
| | GGGGSQIVLIQSPATLSLSPGERATLTCSASSSVSYMHWYQQKPGLSPKR |
| | WIYDTSKLASGVPDRFSGSGSGTDYTFTIRRLEPEDFATYYCQQWSSNPL |
| | TFGQGTKVEIKRAAAPTTTPAPRPPTPAPTIASQPLSLRPEACRPAAGGA |
| | VHIRGLDFACDIYIWAPLAGTCGVLLLSLVITLYCNRDQRLPPDAHKPPG |
| | GGSFRTPIQEEQADAHSTLAKIGSGEGRGSLLTCGDVEENPGPMYRMQLL |
| | SCIALSLALVINSATSNWVNVISDLKKIEDLIQSMHIDATLYTESDVHPS |
| | CKVTAMKCFLLELQVISLESGDASIHDTVENLIILANNSLSSNGNVTESG |
| | CKECEELEEKNIKEFLQSFVHIVQMFINTS |
| 82 | MDWTWRILFLVAAATGAHSQVQLQQSGAEVKKPGASVKVSCKASGYTFTN |
| | YWMQWVRQAPGQGLEWIGAVYPGDGDTRHTQKFKGRVTMTADKSTSTVYM |
| | ELSSLRSEDTAVYYCARGFTAYGMDYWGQGTLVTVSSASTGGGGSGGGGS |
| | GGGGSQIVLTQSPATLSLSPGERATLTCSASSSVSYMHWYQQKPGLSPKR |
| | WIYDISKLASGVPDRFSGSGSGTDYTFTIRRLEPEDFATYYCQQWSSNPL |
| | TFGQGTKVEIKRAAAIEVMYPPPYLDNEKSNGTIIHVKGKHLCPSPLFPG |
| | PSKPKDPKFWVLVVVGGVLACYSLLVTVAFIIFWVALYLLRRDQRLPPDA |
| | HKPPGGGSFRTPIQEEQADAHSTLAKIGPQCTNYALLKLAGDVESNPGPM |
| | RISKPHLRSISIQCYLCLLLNSHELTEAGIHVFILGCFSAGLPKTEANWV |
| | NVISDLKKIEDLIQSMHIDATLYTESDVHPSCKVTAMKCFLLELQVISLE |
| | SGDASIHDTVENLIILANNSLSSNGNVTESGCKECEELEEKNIKEFLQSF |
| | VHIVQMFINTS |

In some embodiments, the ADGRE2 targeting CAR or CCR polypeptide construct comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical, or 100% identical to any one of amino acid sequences SEQ ID NO: 48, or 72-82.

In some embodiments, the ADGRE2 targeting CAR or CCR polypeptide construct comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical, or 100% identical to any one of amino acid sequences SEQ ID NO: 48.

In some embodiments, the ADGRE2 targeting CAR or CCR polypeptide construct comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical, or 100% identical to any one of amino acid sequences SEQ ID NO: 72.

In some embodiments, the ADGRE2 targeting CAR or CCR polypeptide construct comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical, or 100% identical to any one of amino acid sequences SEQ ID NO: 73.

In some embodiments, the ADGRE2 targeting CAR or CCR polypeptide construct comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical, or 100% identical to any one of amino acid sequences SEQ ID NO: 74.

In some embodiments, the ADGRE2 targeting CAR or CCR polypeptide construct comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical, or 100% identical to any one of amino acid sequences SEQ ID NO: 75.

In some embodiments, the ADGRE2 targeting CAR or CCR polypeptide construct comprises amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical, or 100% identical to any one of amino acid sequences SEQ ID NO: 76.

In some embodiments, the ADGRE2 targeting CAR or CCR polypeptide construct comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical, or 100% identical to any one of amino acid sequences SEQ ID NO: 77.

In some embodiments, the ADGRE2 targeting CAR or CCR polypeptide construct comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical, or 100% identical to any one of amino acid sequences SEQ ID NO: 78.

In some embodiments, the ADGRE2 targeting CAR or CCR polypeptide construct comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical, or 100% identical to any one of amino acid sequences SEQ ID NO: 79.

In some embodiments, the ADGRE2 targeting CAR or CCR polypeptide construct comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical, or 100% identical to any one of amino acid sequences SEQ ID NO: 80.

In some embodiments, the ADGRE2 targeting CAR or CCR polypeptide construct comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical, or 100% identical to any one of amino acid sequences SEQ ID NO: 81.

In some embodiments, the ADGRE2 targeting CAR or CCR polypeptide construct comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical, or 100% identical to any one of amino acid sequences SEQ ID NO: 82.

### Polynucleotides Encoding ADGRE2 Targeting Chimeric Receptors

In another aspect, the present invention provides nucleic acid molecules encoding one or more chimeric receptor polypeptides as described herein. As used herein, the terms “nucleic acid molecule” and “polynucleotide” are used interchangeably. In some embodiments, the nucleic acid molecule is provided as a messenger RNA (mRNA) molecule. In other embodiments, the nucleic acid molecule is provided as a DNA construct. In some aspects, the DNA construct is a non-viral vector such as a plasmid, a cosmid or an artificial chromosome. In other aspects, the DNA construct is a viral based vector such as AAV, lentivirus and retrovirus.

Accordingly, the present invention provides a polynucleotide encoding a ADGRE2 chimeric receptor polypeptide (e.g., a CAR or CCR). In some embodiments, the chimeric receptor is a CAR comprising an ADGRE2 binding domain (e.g., a human anti-ADGRE2 binding domain), a hinge, a transmembrane domain, a DAP10 costimulatory domain and an intracellular signaling domain comprising a primary signaling domain of CD3ξ. In some embodiments, the polynucleotide further comprises a nucleic acid sequence encoding one or more polypeptides that are incorporated into the CAR construct. For example, the polynucleotide further comprises a nucleic acid sequence encoding a cytokine such as IL-15. In some embodiments, the polynucleotide further comprises nucleic acid sequences for a single peptide and/or a linker sequence (e.g., E2A).

In some embodiments, the chimeric receptor is a CCR comprising an ADGRE2 binding domain (e.g., a human anti-ADGRE2 binding domain), a hinge, a transmembrane domain, and an intracellular domain.

In some embodiments, the ADGRE2 binding domain is an anti-ADGRE2 binding domain described herein. In some embodiments, the anti-ADGRE2 binding domain is encoded by a nucleic acid sequence of any one of SEQ ID NOs.: 30-35. In some embodiments, the nucleic acid sequence encoding the anti-ADGRE2 binding domain of the chimeric receptor comprises a nucleic acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to one of the sequence of SEQ ID NOs: 30-35. An exemplary nucleic acid sequence encoding the ADGRE2 binding domain comprises SEQ ID NO: 35, or a sequence that is about 70%, 75%, 80%, 85%, 90%, 91%, 92%, 93%, 94%, 95%, 96%, 97%, 98%, or 99% identical to the sequence of SEQ ID NO: 35.

The polynucleotide encoding the CAR may be codon optimized. In some embodiments, the polynucleotide comprises at least one modified nucleotide. In other embodiments, the polynucleotide comprises unmodified nucleotides only.

The polynucleotide encoding the CAR polypeptide of the present invention can be obtained using recombinant methods known in the art, for example by screening libraries from cells expressing the CAR construct, by deriving the CAR construct from a vector known to include the same, or by isolating directly from cells and tissues containing the same, using standard techniques. Alternatively, the polynucleotide can be produced synthetically in vitro.

In alternative embodiments, the polynucleotide sequence encoding various components of a ADGRE2 targeting CAR can be disposed on the different nucleic acid molecules, e.g., different plasmids or vectors, e.g., viral vector, e.g., lentiviral vector. For example, the (i) sequence encoding an antigen binding member can be present on a first nucleic acid, e.g., a first vector, and the (ii) sequence encoding an intracellular signaling member can be present on the second nucleic acid, e.g., the second vector.

In some embodiments, the polynucleotide encoding the ADGRE2 chimeric receptor comprises a sequence that is at least 70%, 75%, 80%, 85%, 90%, 91%, 92%, 93%, 94%, 95%, 96%, 97%, 98%, 99% identical or identical to SEQ ID NO: 49 and 83-86.

In some embodiments, the polynucleotide encoding the ADGRE2 chimeric receptor comprises a sequence that is at least 70%, 75%, 80%, 85%, 90%, 91%, 92%, 93%, 94%, 95%, 96%, 97%, 98%, 99% identical or identical to SEQ ID NO: 49.

In some embodiments, the polynucleotide encoding the ADGRE2 chimeric receptor comprises a sequence that is at least 70%, 75%, 80%, 85%, 90%, 91%, 92%, 93%, 94%, 95%, 96%, 97%, 98%, 99% identical or identical to SEQ ID NO: 83.

In some embodiments, the polynucleotide encoding the ADGRE2 chimeric receptor comprises a sequence that is at least 70%, 75%, 80%, 85%, 90%, 91%, 92%, 93%, 94%, 95%, 96%, 97%, 98%, 99% identical or identical to SEQ ID NO: 84.

In some embodiments, the polynucleotide encoding the ADGRE2 chimeric receptor comprises a sequence that is at least 70%, 75%, 80%, 85%, 90%, 91%, 92%, 93%, 94%, 95%, 96%, 97%, 98%, 99% identical or identical to SEQ ID NO: 85.

In some embodiments, the polynucleotide encoding the ADGRE2 chimeric receptor comprises a sequence that is at least 70%, 75%, 80%, 85%, 90%, 91%, 92%, 93%, 94%, 95%, 96%, 97%, 98%, 99% identical or identical to SEQ ID NO: 86.

| SEQ ID NO: | Sequence |
|---|---|
| 49 | TGAAAGACCCCACCTGTAGGTTTGGCAAGCTAGCTTAAGTAACGCCATTTT |
| | GCAAGGCATGGAAAAATACATAACTGAGAATAGAAAAGTTCAGATCAAGGT |
| | CAGGAACAGATGGAACAGCTGAATATGGGCCAAACAGGATATCTGTGGTAA |
| | GCAGTTCCTGCCCCGGCTCAGGGCCAAGAACAGATGGAACAGCTGAATATG |
| | GGCCAAACAGGATATCTGTGGTAAGCAGTTCCTGCCCCGGCTCAGGGCCAA |
| | GAACAGATGGTCCCCAGATGCGGTCCAGCCCTCAGCAGTTTCTAGAGAACC |
| | ATCAGATGTTTCCAGGGTGCCCCAAGGACCTGAAATGACCCTGTGCCTTAT |
| | TTGAACTAACCAATCAGTTCGCTTCTCGCTTCTGTTCGCGCGCTTATGCTC |
| | CCCGAGCTCAATAAAAGAGCCCACAACCCCTCACTCGGGGCGCCAGTCCTC |
| | CGATTGACTGAGTCGCCCGGGTACCCGTGTATCCAATAAACCCTCTTGCAG |
| | TTGCATCCGACTTGTGGTCTCGCTGTTCCTTGGGAGGGTCTCCTCTGAGTG |
| | ATTGACTACCCGTCAGCGGGGGTCTTTCATTTGGGGGCTCGTCCGGGATCG |
| | GGAGACCCCTGCCCAGGGACCACCGACCCACCACCGGGAGGTAAGCTGGCC |
| | AGCAACTTATCTGTGTCTGTCCGATTGTCTAGTGTCTATGACTGATTTTAT |
| | GCGCCTGCGTCGGTACTAGTTAGCTAACTAGCTCTGTATCTGGCGGACCCG |
| | TGGTGGAACTGACGAGTTCGGAACACCCGGCCGCAACCCTGGGAGACGTCC |
| | CAGGGACTTCGGGGGCCGTTTTTGTGGCCCGACCTGAGTCCTAAAATCCCG |
| | ATCGTTTAGGACTCTTTGGTGCACCCCCCTTAGAGGAGGGATATGTGGTTC |
| | TGGTAGGAGACGAGAACCTAAAACAGTTCCCGCCTCCGTCTGAATTTTTGC |
| | TTTCGGTTTGGGACCGAAGCCGCGCCGCGCGTCTTGTCTGCTGCAGCATCG |
| | TTCTGTGTTGTCTCTGTCTGACTGTGTTTCTGTATTTGTCTGAAAATATGG |
| | GCCCGGGCTAGCCTGTTACCACTCCCTTAAGTTTGACCTTAGGTCACTGGA |
| | AAGATGTCGAGCGGATCGCTCACAACCAGTCGGTAGATGTCAAGAAGAGAC |
| | GTTGGGTTACCTTCTGCTCTGCAGAATGGCCAACCTTTAACGTCGGATGGC |
| | CGCGAGACGGCACCTTTAACCGAGACCTCATCACCCAGGTTAAGATCAAGG |
| | TCTTTTCACCTGGCCCGCATGGACACCCAGACCAGGTGGGGTACATCGTGA |
| | CCTGGGAAGCCTTGGCTTTTGACCCCCCTCCCTGGGTCAAGCCCTTTGTAC |
| | ACCCTAAGCCTCCGCCTCCTCTTCCTCCATCCCCCCGTCTCTCCCCCTTGA |
| | ACCTCCTCGTTCGACCCCGCCTCGATCCTCCCTTTATCCAGCCCTCACTCC |
| | TTCTCTAGGCGCCCCCATATGGCCATATGAGATCTTATATGGGGCACCCCC |
| | GCCCCTTGTAAACTTCCCTGACCCTGACATGACAAGAGTTACTAACAGCCC |
| | CTCTCTCCAAGCTCACTTACAGGCTCTCTACTTAGTCCAGCACGAAGTCTG |
| | GAGACCTCTGGCGGCAGCCTACCAAGAACAACTGGACCGACCGGTGGTACC |
| | TCACCCTTACCGAGTCGGCGACACAGTGTGGGTCCGCCGACACCAGACTAA |
| | GAACCTAGAACCTCGCTGGAAAGGACCTTACACAGTCCTGCTGACCACCCC |
| | CACCGCCCTCAAAGTAGACGGCATCGCAGCTTGGATACACGCCGCCCACGT |
| | GAAGGCTGCCGACCCCGGGGGTGGACCATCCTCTAGACTGCCATGCTCGAG |
| | TCGCGACCATGGACTGGACCTGGAGAATCCTGTTCCTGGTGGCCGCCGCTA |
| | CAGGAGCCCACAGCCAGGTGCAGTTGCAGCAGAGCGGCGCTGAGGTGGCCA |
| | AGCCTGGAGCTAGCGTGAAGCTGAGCTGCAAGGCCAGCGGCTACACCTTCA |
| | CCAACTACTGGATGCAGTGGATCAAGCAGGCCCCCGGACAAGGCTTGGAGT |
| | GGATTGGCGCCGTGTACCCCGGAGATGGAGACACCAGGCACACCCAGAAGT |
| | TCAAGGGCAAGGCCACCCTGACAGCCGACAAGAGCACCAGCACCGCCTACA |
| | TGGAGGTGAGCAGCCTGAGGAGCGAGGACACCGCCGTGTACTACTGCGCCA |
| | GGGGCTTCACCGCCTACGGCATGGACTATTGGGGCCAGGGCACCACAGTGA |
| | CCGTGAGCTCTGGAGGAGGAGGCAGCGGAGGAGGAGGATCTGGAGGAGGCG |
| | GAAGCGAGATCGTGCTGACCCAGAGCCCTGCCACCATGAGCGCTTCTCCCG |
| | GCGAAAGGGTGACCATGAGCTGCAGCGCCAGCAGCAGCGTGAGCTACATGC |
| | ACTGGTACCAGCAGAAGAGCGGCCAGAGCCCCAAGAGGTGGATCTACGACA |
| | CCAGCAAGCTGGCCAGCGGCGTGCCTGCCAGATTCTCTGGCAGCGGATCTG |
| | GCACCGACTACACATTCACCATCAGCAGCATGGAGCCCGAGGACTTCGCCA |
| | CCTACTACTGCCAGCAGTGGAGCAGCAACCCCCTGACCTTCGGAGGCGGCA |
| | CCAAGCTGGAGATCAAGAGGGCCGCCGCCATCGAGGTGATGTATCCCCCTC |
| | CCTACCTGGACAACGAGAAGAGCAACGGCACCATCATCCACGTGAAGGGCA |
| | AGCACCTGTGCCCCAGCCCTCTGTTCCCTGGCCCTAGCAAGCCCAAGGACC |
| | CCAAGTTCTGGGTGCTCGTGGTGGTGGGAGGAGTGCTGGCCTGCTACAGCC |
| | TGCTGGTGACCGTGGCCTTCATCATCTTCTGGGTGCTGTGCGCCAGGCCCA |
| | GAAGATCTCCTGCCCAGGAGGACGGCAAGGTGTACATCAACATGCCCGGCA |
| | GGGGCAGGGTGAAGTTCTCTAGGAGCGCTGACGCCCCTGCTTACCAGCAGG |
| | GCCAGAACCAGCTGTATAACGAGCTGAACCTGGGCAGAAGAGAGGAGTACG |
| | ACGTGCTGGACAAGAGGAGGGGCAGGGACCCCGAAATGGGCGGCAAGCCCA |
| | GAAGGAAGAACCCCCAGGAGGGCCTGTACAACGAGCTGCAGAAGGACAAGA |
| | TGGCCGAGGCCTACAGCGAGATCGGCATGAAGGGCGAGAGGAGAAGAGGCA |
| | AGGGCCACGACGGCCTGTACCAGGGCCTGAGCACAGCTACCAAGGACACCT |
| | ACGACGCCCTGCACATGCAAGCCCTTCCCCCCAGAGGACCCCAGTGCACCA |
| | ACTACGCCCTGCTGAAGCTGGCCGGCGACGTGGAAAGCAACCCTGGCCCCA |
| | TGAGAATCAGCAAGCCCCACCTGAGGAGCATCAGCATCCAGTGCTACCTGT |
| | GCCTGCTGCTGAACAGCCACTTCCTGACCGAGGCCGGCATCCACGTGTTCA |
| | TCCTGGGCTGCTTCAGCGCCGGCCTGCCCAAGACCGAAGCCAACTGGGTGA |
| | ACGTGATCAGCGACCTGAAGAAGATCGAGGACCTGATCCAGAGCATGCACA |
| | TCGACGCCACCCTGTACACCGAGAGCGACGTGCACCCCAGCTGCAAGGTGA |
| | CCGCCATGAAGTGCTTCCTGCTGGAGCTGCAGGTGATCAGCCTGGAGAGCG |
| | GCGACGCCAGCATCCACGACACCGTGGAGAACCTGATCATCCTGGCCAACA |
| | ACAGCCTGAGCAGCAACGGCAACGTGACCGAGAGCGGCTGCAAGGAGTGCG |
| | AGGAGCTGGAGGAGAAGAACATCAAGGAGTTCCTGCAGAGCTTCGTGCACA |
| | TCGTGCAGATGTTCATCAACACCAGCTGACAATTGCGCGTCATCATCGATC |
| | CGGATTAGTCCAATTTGTTAAAGACAGGATATCAGTGGTCCAGGCTCTAGT |
| | TTTGACTCAACAATATCACCAGCTGAAGCCTATAGAGTACGAGCCATAGAT |
| | AAAATAAAAGATTTTATTTAGTCTCCAGAAAAAGGGGGGAATGAAAGACCC |
| | CACCTGTAGGTTTGGCAAGCTAGCTTAAGTAACGCCATTTTGCAAGGCATG |
| | GAAAAATACATAACTGAGAATAGAGAAGTTCAGATCAAGGTCAGGAACAGA |
| | TGGAACAGCTGAATATGGGCCAAACAGGATATCTGTGGTAAGCAGTTCCTG |
| | CCCCGGCTCAGGGCCAAGAACAGATGGAACAGCTGAATATGGGCCAAACAG |
| | GATATCTGTGGTAAGCAGTTCCTGCCCCGGCTCAGGGCCAAGAACAGATGG |
| | TCCCCAGATGCGGTCCAGCCCTCAGCAGTTTCTAGAGAACCATCAGATGTT |
| | TCCAGGGTGCCCCAAGGACCTGAAATGACCCTGTGCCTTATTTGAACTAAC |
| | CAATCAGTTCGCTTCTCGCTTCTGTTCGCGCGCTTCTGCTCCCCGAGCTCA |
| | ATAAAAGAGCCCACAACCCCTCACTCGGGGCGCCAGTCCTCCGATTGACTG |
| | AGTCGCCCGGGTACCCGTGTATCCAATAAACCCTCTTGCAGTTGCATCCGA |
| | CTTGTGGTCTCGCTGTTCCTTGGGAGGGTCTCCTCTGAGTGATTGACTACC |
| | CGTCAGCGGGGGTCTTTCACACATGCAGCATGTATCAAAATTAATTTGGTT |
| | TTTTTTCTTAAGTATTTACATTAAATGGCCATAGTACTTAAAGTTACATTG |
| | GCTTCCTTGAAATAAACATGGAGTATTCAGAATGTGTCATAAATATTTCTA |
| | ATTTTAAGATAGTATCTCCATTGGCTTTCTACTTTTTCTTTTATTTTTTTT |
| | TGTCCTCTGTCTTCCATTTGTTGTTGTTGTTGTTTGTTTGTTTGTTTGTTG |
| | GTTGGTTGGTTAATTTTTTTTTAAAGATCCTACACTATAGTTCAAGCTAGA |
| | CTATTAGCTACTCTGTAACCCAGGGTGACCTTGAAGTCATGGGTAGCCTGC |
| | TGTTTTAGCCTTCCCACATCTAAGATTACAGGTATGAGCTATCATTTTTGG |
| | TATATTGATTGATTGATTGATTGATGTGTGTGTGTGTGATTGTGTTTGTGT |
| | GTGTGACTGTGAAAATGTGTGTATGGGTGTGTGTGAATGTGTGTATGTATG |
| | TGTGTGTGTGAGTGTGTGTGTGTGTGTGTGCATGTGTGTGTGTGTGACTGT |
| | GTCTATGTGTATGACTGTGTGTGTGTGTGTGTGTGTGTGTGTGTGTGTGTG |
| | TGTTGTGAAAAAATATTCTATGGTAGTGAGAGCCAACGCTCCGGCTCAGGT |
| | GTCAGGTTGGTTTTTGAGACAGAGTCTTTCACTTAGCTTGGAATTCACTGG |
| | CCGTCGTTTTACAACGTCGTGACTGGGAAAACCCTGGCGTTACCCAACTTA |
| | ATCGCCTTGCAGCACATCCCCCTTTCGCCAGCTGGCGTAATAGCGAAGAGG |
| | CCCGCACCGATCGCCCTTCCCAACAGTTGCGCAGCCTGAATGGCGAATGGC |
| | GCCTGATGCGGTATTTTCTCCTTACGCATCTGTGCGGTATTTCACACCGCA |
| | TATGGTGCACTCTCAGTACAATCTGCTCTGATGCCGCATAGTTAAGCCAGC |
| | CCCGACACCCGCCAACACCCGCTGACGCGCCCTGACGGGCTTGTCTGCTCC |
| | CGGCATCCGCTTACAGACAAGCTGTGACCGTCTCCGGGAGCTGCATGTGTC |
| | AGAGGTTTTCACCGTCATCACCGAAACGCGCGAGACGAAAGGGCCTCGTGA |
| | TACGCCTATTTTTATAGGTTAATGTCATGATAATAATGGTTTCTTAGACGT |
| | CAGGTGGCACTTTTCGGGGAAATGTGCGCGGAACCCCTATTTGTTTATTTT |
| | TTTAAATACATTCAAATATGTATCCGCTCATGAGACAATAACCCTGATAAA |
| | TGCTTCAATAATATTGAAAAAGGAAGAGTATGAGCCATATTCAACGGGAAA |
| | CGTCGAGGCCGCGATTAAATTCCAACATGGATGCTGATTTATATGGGTATA |
| | AATGGGCTCGCGATAATGTCGGGCAATCAGGTGCGACAATCTATCGCTTGT |
| | ATGGGAAGCCCGATGCGCCAGAGTTGTTTCTGAAACATGGCAAAGGTAGCG |
| | TTGCCAATGATGTTACAGATGAGATGGTCAGACTAAACTGGCTGACGGAAT |
| | TTATGCCTCTTCCGACCATCAAGCATTTTATCCGTACTCCTGATGATGCAT |
| | GGTTACTCACCACTGCGATCCCCGGAAAAACAGCATTCCAGGTATTAGAAG |
| | AATATCCTGATTCAGGTGAAAATATTGTTGATGCGCTGGCAGTGTTCCTGC |
| | GCCGGTTGCATTCGATTCCTGTTTGTAATTGTCCTTTTAACAGCGATCGCG |
| | TATTTCGTCTCGCTCAGGCGCAATCACGAATGAATAACGGTTTGGTTGATG |
| | CGAGTGATTTTGATGACGAGCGTAATGGCTGGCCTGTTGAACAAGTCTGGA |
| | AAGAAATGCATAAACTTTTGCCATTCTCACCGGATTCAGTCGTCACTCATG |
| | GTGATTTCTCACTTGATAACCTTATTTTTGACGAGGGGAAATTAATAGGTT |
| | GTATTGATGTTGGACGAGTCGGAATCGCAGACCGATACCAGGATCTTGCCA |
| | TCCTATGGAACTGCCTCGGTGAGTTTTCTCCTTCATTACAGAAACGGCTTT |
| | TTCAAAAATATGGTATTGATAATCCTGATATGAATAAATTGCAGTTTCATT |
| | TGATGCTCGATGAGTTTTTCTAACTGTCAGACCAAGTTTACTCATATATAC |
| | TTTAGATTGATTTAAAACTTCATTTTTAATTTAAAAGGATCTAGGTGAAGA |
| | TCCTTTTTGATAATCTCATGACCAAAATCCCTTAACGTGAGTTTTCGTTCC |
| | ACTGAGCGTCAGACCCCGTAGAAAAGATCAAAGGATCTTCTTGAGATCCTT |
| | TTTTTCTGCGCGTAATCTGCTGCTTGCAAACAAAAAAACCACCGCTACCAG |
| | CGGTGGTTTGTTTGCCGGATCAAGAGCTACCAACTCTTTTTCCGAAGGTAA |
| | CTGGCTTCAGCAGAGCGCAGATACCAAATACTGTTCTTCTAGTGTAGCCGT |
| | AGTTAGGCCACCACTTCAAGAACTCTGTAGCACCGCCTACATACCTCGCTC |
| | TGCTAATCCTGTTACCAGTGGCTGCTGCCAGTGGCGATAAGTCGTGTCTTA |
| | CCGGGTTGGACTCAAGACGATAGTTACCGGATAAGGCGCAGCGGTCGGGCT |
| | GAACGGGGGGTTCGTGCACACAGCCCAGCTTGGAGCGAACGACCTACACCG |
| | AACTGAGATACCTACAGCGTGAGCTATGAGAAAGCGCCACGCTTCCCGAAG |
| | GGAGAAAGGCGGACAGGTATCCGGTAAGCGGCAGGGTCGGAACAGGAGAGC |
| | GCACGAGGGAGCTTCCAGGGGGAAACGCCTGGTATCTTTATAGTCCTGTCG |
| | GGTTTCGCCACCTCTGACTTGAGCGTCGATTTTTGTGATGCTCGTCAGGGG |
| | GGCGGAGCCTATGGAAAAACGCCAGCAACGCGGCCTTTTTACGGTTCCTGG |
| | CCTTTTGCTGGCCTTTTGCTCACATGTTCTTTCCTGCGTTATCCCCTGATT |
| | CTGTGGATAACCGTATTACCGCCTTTGAGTGAGCTGATACCGCTCGCCGCA |
| | GCCGAACGACCGAGCGCAGCGAGTCAGTGAGCGAGGAAGCGGAAGAGCGCC |
| | CAATACGCAAACCGCCTCTCCCCGCGCGTTGGCCGATTCATTAATGCAGCT |
| | GGCACGACAGGTTTCCCGACTGGAAAGCGGGCAGTGAGCGCAACGCAATTA |
| | ATGTGAGTTAGCTCACTCATTAGGCACCCCAGGCTTTACACTTTATGCTTC |
| | CGGCTCGTATGTTGTGTGGAATTGTGAGCGGATAACAATTTCACACAGGAA |
| | ACAGCTATGACCATGATTACGCCAAGCTTTGCTCTTAGGAGTTTCCTAATA |
| | CATCCCAAACTCAAATATATAAAGCATTTGACTTGTTCTATGCCCTAGGGG |
| | GCGGGGGGAAGCTAAGCCAGCTTTTTTTAACATTTAAAATGTTAATTCCAT |
| | TTTAAATGCACAGATGTTTTTATTTCATAAGGGTTTCAATGTGCATGAATG |
| | CTGCAATATTCCTGTTACCAAAGCTAGTATAAATAAAAATAGATAAACGTG |
| | GAAATTACTTAGAGTTTCTGTCATTAACGTTTCCTTCCTCAGTTGACAACA |
| | TAAATGCGCTGCTGAGCAAGCCAGTTTGCATCTGTCAGGATCAATTTCCCA |
| | TTATGCCAGTCATATTAATTACTAGTCAATTAGTTGATTTTTATTTTTGAC |
| | ATATACATGTGAA |
| 83 | ATGGACTGGACCTGGAGAATCCTGTTCCTGGTGGCCGCCGCTACAGGAGCC |
| | CACAGCCAGGTGCAGTTGCAGCAGAGCGGCGCTGAGGTGGCCAAGCCTGGA |
| | GCTAGCGTGAAGCTGAGCTGCAAGGCCAGCGGCTACACCTTCACCAACTAC |
| | TGGATGCAGTGGATCAAGCAGGCCCCCGGACAAGGCTTGGAGTGGATTGGC |
| | GCCGTGTACCCCGGAGATGGAGACACCAGGCACACCCAGAAGTTCAAGGGC |
| | AAGGCCACCCTGACAGCCGACAAGAGCACCAGCACCGCCTACATGGAGGTG |
| | AGCAGCCTGAGGAGCGAGGACACCGCCGTGTACTACTGCGCCAGGGGCTTC |
| | ACCGCCTACGGCATGGACTATTGGGGCCAGGGCACCACAGTGACCGTGAGC |
| | TCTGGAGGAGGAGGCAGCGGAGGAGGAGGATCTGGAGGAGGCGGAAGCGAG |
| | ATCGTGCTGACCCAGAGCCCTGCCACCATGAGCGCTTCTCCCGGCGAAAGG |
| | GTGACCATGAGCTGCAGCGCCAGCAGCAGCGTGAGCTACATGCACTGGTAC |
| | CAGCAGAAGAGCGGCCAGAGCCCCAAGAGGTGGATCTACGACACCAGCAAG |
| | CTGGCCAGCGGCGTGCCTGCCAGATTCTCTGGCAGCGGATCTGGCACCGAC |
| | TACACATTCACCATCAGCAGCATGGAGCCCGAGGACTTCGCCACCTACTAC |
| | TGCCAGCAGTGGAGCAGCAACCCCCTGACCTTCGGAGGCGGCACCAAGCTG |
| | GAGATCAAGAGGGCCGCCGCCATCGAGGTGATGTATCCCCCTCCCTACCTG |
| | GACAACGAGAAGAGCAACGGCACCATCATCCACGTGAAGGGCAAGCACCTG |
| | TGCCCCAGCCCTCTGTTCCCTGGCCCTAGCAAGCCCAAGGACCCCAAGTTC |
| | TGGGTGCTCGTGGTGGTGGGAGGAGTGCTGGCCTGCTACAGCCTGCTGGTG |
| | ACCGTGGCCTTCATCATCTTCTGGGTGCTGTGCGCCAGGCCCAGAAGATCT |
| | CCTGCCCAGGAGGACGGCAAGGTGTACATCAACATGCCCGGCAGGGGCAGG |
| | GTGAAGTTCTCTAGGAGCGCTGACGCCCCTGCTTACCAGCAGGGCCAGAAC |
| | CAGCTGTATAACGAGCTGAACCTGGGCAGAAGAGAGGAGTACGACGTGCTG |
| | GACAAGAGGAGGGGCAGGGACCCCGAAATGGGCGGCAAGCCCAGAAGGAAG |
| | AACCCCCAGGAGGGCCTGTACAACGAGCTGCAGAAGGACAAGATGGCCGAG |
| | GCCTACAGCGAGATCGGCATGAAGGGCGAGAGGAGAAGAGGCAAGGGCCAC |
| | GACGGCCTGTACCAGGGCCTGAGCACAGCTACCAAGGACACCTACGACGCC |
| | CTGCACATGCAAGCCCTTCCCCCCAGAGGACCCCAGTGCACCAACTACGCC |
| | CTGCTGAAGCTGGCCGGCGACGTGGAAAGCAACCCTGGCCCCATGAGAATC |
| | AGCAAGCCCCACCTGAGGAGCATCAGCATCCAGTGCTACCTGTGCCTGCTG |
| | CTGAACAGCCACTTCCTGACCGAGGCCGGCATCCACGTGTTCATCCTGGGC |
| | TGCTTCAGCGCCGGCCTGCCCAAGACCGAAGCCAACTGGGTGAACGTGATC |
| | AGCGACCTGAAGAAGATCGAGGACCTGATCCAGAGCATGCACATCGACGCC |
| | ACCCTGTACACCGAGAGCGACGTGCACCCCAGCTGCAAGGTGACCGCCATG |
| | AAGTGCTTCCTGCTGGAGCTGCAGGTGATCAGCCTGGAGAGCGGCGACGCC |
| | AGCATCCACGACACCGTGGAGAACCTGATCATCCTGGCCAACAACAGCCTG |
| | AGCAGCAACGGCAACGTGACCGAGAGCGGCTGCAAGGAGTGCGAGGAGCTG |
| | GAGGAGAAGAACATCAAGGAGTTCCTGCAGAGCTTCGTGCACATCGTGCAG |
| | ATGTTCATCAACACCAGC |
| 84 | ATGGACTGGACCTGGAGAATCCTGTTCCTGGTTGCTGCTGCTACAGGAGCC |
| | CATAGCCAGGTGCAACTGCAACAAAGCGGAGCAGAGGTGGCTAAACCCGGA |
| | GCTAGCGTGAAGTTGAGCTGTAAGGCCAGCGGCTACACATTCACCAACTAC |
| | TGGATGCAGTGGATCAAACAGGCCCCTGGACAAGGATTGGAGTGGATTGGC |
| | GCTGTGTATCCTGGCGATGGAGATACAAGGCACACCCAGAAGTTCAAGGGC |
| | AAAGCCACCTTGACAGCCGACAAGAGCACAAGCACCGCCTACATGGAGGTG |
| | AGCTCTCTGAGGAGCGAAGATACCGCCGTGTACTATTGTGCCAGGGGCTTC |
| | ACAGCTTACGGCATGGACTATTGGGGCCAGGGAACAACCGTTACCGTTAGC |
| | TCTGGTGGAGGTGGATCAGGTGGAGGAGGATCAGGAGGAGGTGGTAGCGAG |
| | ATCGTGCTGACACAATCTCCCGCCACCATGTCTGCTTCACCCGGAGAGAGA |
| | GTGACAATGAGCTGCTCTGCTTCTAGCAGCGTGAGCTACATGCATTGGTAT |
| | CAGCAGAAGAGCGGACAGTCTCCCAAAAGATGGATCTACGACACCAGCAAG |
| | CTGGCTTCTGGAGTTCCTGCTAGGTTCTCTGGATCTGGAAGCGGCACAGAC |
| | TACACCTTCACCATCAGCAGCATGGAACCCGAGGACTTCGCTACCTATTAC |
| | TGCCAGCAGTGGAGCTCTAACCCTCTGACATTCGGAGGAGGCACCAAGTTG |
| | GAGATCAAGAGAGCTGCTGCTCCCACCACAACACCCGCTCCTAGGCCTCCT |
| | ACACCAGCCCCTACCATCGCCTCTCAGCCCCTGTCTCTTAGGCCTGAGGCC |
| | TGTAGACCTGCTGCTGGAGGAGCTGTGCACACAAGGGGCCTGGATTTTGCC |
| | TGCGACATCTACATCTGGGCTCCCTTGGCTGGAACCTGCGGAGTGCTGCTG |
| | CTGTCTCTGGTGATCACCCTGTACTGCAACCTGTGCGCCAGACCCAGAAGA |
| | TCTCCTGCACAGGAAGACGGCAAGGTGTACATCAACATGCCCGGCAGAGGC |
| | AGGGTGAAGTTCTCTAGGAGTGCAGACGCTCCTGCTTACCAGCAAGGCCAG |
| | AACCAGCTCTACAACGAGCTGAATCTGGGACGCAGAGAGGAGTACGATGTG |
| | CTGGACAAACGGAGGGGAAGAGATCCTGAGATGGGAGGCAAACCCAGACGG |
| | AAGAACCCTCAAGAGGGCCTGTACAATGAGCTGCAGAAGGACAAGATGGCC |
| | GAAGCCTACAGCGAGATCGGAATGAAGGGCGAAAGAAGGAGAGGAAAAGGC |
| | CACGATGGGCTGTATCAAGGCCTGAGCACAGCTACCAAGGACACCTACGAC |
| | GCTCTGCACATGCAAGCTCTGCCTCCTAGAGGCCCCCAGTGTACCAACTAC |
| | GCCTTGCTGAAACTGGCTGGCGACGTGGAATCTAACCCCGGACCCATGCGG |
| | ATCAGCAAGCCCCATCTGAGGTCCATCAGCATCCAGTGCTACCTGTGCCTG |
| | CTGCTGAACAGCCACTTCCTCACCGAAGCCGGCATCCACGTGTTCATTCTG |
| | GGCTGCTTCTCTGCTGGCCTGCCCAAGACAGAGGCTAACTGGGTCAACGTG |
| | ATCAGCGACCTGAAGAAGATCGAGGACCTGATCCAGAGCATGCACATTGAC |
| | GCCACCCTGTACACCGAGAGCGACGTTCACCCCTCTTGCAAGGTGACCGCC |
| | ATGAAGTGCTTCCTGCTTGAGCTGCAGGTGATTAGCCTGGAGAGCGGAGAC |
| | GCTTCCATCCATGACACCGTGGAGAACCTGATTATCCTGGCCAACAACAGC |
| | CTGAGCAGCAACGGCAATGTGACCGAAAGCGGCTGCAAGGAATGCGAGGAG |
| | CTCGAGGAGAAGAACATCAAGGAGTTCCTGCAGAGCTTCGTGCACATCGTG |
| | CAGATGTTCATCAACACCAGC |
| 85 | ATGGACTGGACTTGGAGAATATTGTTTTTGGTTGCTGCTGCTACAGGCGCT |
| | CACTCACAGGTGCAGTTGCAGCAGAGCGGCGCTGAGGTGGCCAAGCCTGGA |
| | GCTAGCGTGAAGCTGAGCTGCAAGGCCAGCGGCTACACCTTCACCAACTAC |
| | TGGATGCAGTGGATCAAGCAGGCCCCCGGACAAGGCTTGGAGTGGATTGGC |
| | GCCGTGTACCCCGGAGATGGAGACACCAGGCACACCCAGAAGTTCAAGGGC |
| | AAGGCCACCCTGACAGCCGACAAGAGCACCAGCACCGCCTACATGGAGGTG |
| | AGCAGCCTGAGGAGCGAGGACACCGCCGTGTACTACTGCGCCAGGGGCTTC |
| | ACCGCCTACGGCATGGACTATTGGGGCCAGGGCACCACAGTGACCGTGAGC |
| | TCTGGAGGAGGAGGCAGCGGAGGAGGAGGATCTGGAGGAGGCGGAAGCGAG |
| | ATCGTGCTGACCCAGAGCCCTGCCACCATGAGCGCTTCTCCCGGCGAAAGG |
| | GTGACCATGAGCTGCAGCGCCAGCAGCAGCGTGAGCTACATGCACTGGTAC |
| | CAGCAGAAGAGCGGCCAGAGCCCCAAGAGGTGGATCTACGACACCAGCAAG |
| | CTGGCCAGCGGCGTGCCTGCCAGATTCTCTGGCAGCGGATCTGGCACCGAC |
| | TACACATTCACCATCAGCAGCATGGAGCCCGAGGACTTCGCCACCTACTAC |
| | TGCCAGCAGTGGAGCAGCAACCCCCTGACCTTCGGAGGCGGCACCAAGCTG |
| | GAGATCAAGCGGgcggccgcaCCGACAACAACCCCTGCCCCGAGGCCCCCA |
| | ACACCAGCCCCTACAATAGCTAGCCAGCCATTGTCCCTCAGACCCGAGGCC |
| | TGCCGGCCTGCTGCTGGCGGGGCAGTGCACACACGGGGCCTGGACTTTGCT |
| | TGCGACATCTATATTTGGGCCCCGCTGGCGGGAACTTGCGGCGTTCTGCTC |
| | CTGAGTTTGGTTATCACCTTGTACTGTaacAGAGACCAAAGACTGCCACCC |
| | GATGCCCATAAACCCCCAGGAGGAGGATCATTTAGAACACCAATACAAGAA |
| | GAACAAGCCGACGCCCACTCAACCCTCGCTAAGATCGGATCTGGCGAGGGA |
| | AGAGGATCTCTGCTGACCTGTGGCGATGTGGAGGAGAATCCAGGCCCTATG |
| | TATAGAATGCAACTTCTCTCTTGTATAGCTCTGTCCCTTGCTCTTGTTACT |
| | AATAGCGCTACCTCCAACTGGGTTAACGTTATCTCTGACCTCAAAAAAATC |
| | GAGGATCTGATACAATCTATGCACATTGATGCTACCCTCTATACTGAGTCC |
| | GATGTCCATCCCTCCTGTAAAGTGACTGCTATGAAGTGTTTCCTCCTCGAA |
| | CTGCAAGTTATCTCCCTGGAATCCGGCGATGCTTCTATACATGACACCGTT |
| | GAAAACCTCATAATCCTGGCAAATAATTCCCTCTCTAGCAATGGCAACGTC |
| | ACAGAATCCGGCTGTAAGGAATGTGAAGAACTGGAAGAGAAAAATATTAAG |
| | GAGTTCCTGCAGTCTTTCGTGCACATAGTTCAGATGTTCATCAACACTTCT |
| 86 | ATGGACTGGACCTGGAGAATCCTGTTCCTGGTTGCTGCTGCTACAGGAGCC |
| | CATAGCCAGGTGCAACTGCAACAAAGCGGAGCAGAGGTGGCTAAACCCGGA |
| | GCTAGCGTGAAGTTGAGCTGTAAGGCCAGCGGCTACACATTCACCAACTAC |
| | TGGATGCAGTGGATCAAACAGGCCCCTGGACAAGGATTGGAGTGGATTGGC |
| | GCTGTGTATCCTGGCGATGGAGATACAAGGCACACCCAGAAGTTCAAGGGC |
| | AAAGCCACCTTGACAGCCGACAAGAGCACAAGCACCGCCTACATGGAGGTG |
| | AGCTCTCTGAGGAGCGAAGATACCGCCGTGTACTATTGTGCCAGGGGCTTC |
| | ACAGCTTACGGCATGGACTATTGGGGCCAGGGAACAACCGTTACCGTTAGC |
| | TCTGGTGGAGGTGGATCAGGTGGAGGAGGATCAGGAGGAGGTGGTAGCGAG |
| | ATCGTGCTGACACAATCTCCCGCCACCATGTCTGCTTCACCCGGAGAGAGA |
| | GTGACAATGAGCTGCTCTGCTTCTAGCAGCGTGAGCTACATGCATTGGTAT |
| | CAGCAGAAGAGCGGACAGTCTCCCAAAAGATGGATCTACGACACCAGCAAG |
| | CTGGCTTCTGGAGTTCCTGCTAGGTTCTCTGGATCTGGAAGCGGCACAGAC |
| | TACACCTTCACCATCAGCAGCATGGAACCCGAGGACTTCGCTACCTATTAC |
| | TGCCAGCAGTGGAGCTCTAACCCTCTGACATTCGGAGGAGGCACCAAGTTG |
| | GAGATCAAGAGAGCTGCTGCTATCGAGGTGATGTATCCCCCTCCCTACCTG |
| | GACAACGAGAAGAGCAACGGCACCATCATCCACGTGAAGGGCAAGCACCTG |
| | TGCCCCAGCCCTCTGTTCCCTGGCCCTAGCAAGCCCAAGGACCCCAAGTTC |
| | TGGGTGCTGGTGGTGGTGGGAGGAGTGCTGGCCTGCTACAGCCTGCTGGTG |
| | ACCGTGGCCTTCATCATCTTCTGGGTGGCCCTGTATCTTCTGCGGAGGGAT |
| | CAGAGACTTCCCCCTGACGCTCACAAACCTCCTGGAGGCGGTAGCTTTAGG |
| | ACACCCATCCAAGAGGAGCAGGCTGATGCTCACTCCACACTGGCCAAGATC |
| | GGCCCCCAGTGTACCAACTACGCCTTGCTGAAACTGGCTGGCGACGTGGAA |
| | TCTAACCCCGGACCCATGCGGATCAGCAAGCCCCATCTGAGGTCCATCAGC |
| | ATCCAGTGCTACCTGTGCCTGCTGCTGAACAGCCACTTCCTCACCGAAGCC |
| | GGCATCCACGTGTTCATTCTGGGCTGCTTCTCTGCTGGCCTGCCCAAGACA |
| | GAGGCTAACTGGGTCAACGTGATCAGCGACCTGAAGAAGATCGAGGACCTG |
| | ATCCAGAGCATGCACATTGACGCCACCCTGTACACCGAGAGCGACGTTCAC |
| | CCCTCTTGCAAGGTGACCGCCATGAAGTGCTTCCTGCTTGAGCTGCAGGTG |
| | ATTAGCCTGGAGAGCGGAGACGCTTCCATCCATGACACCGTGGAGAACCTG |
| | ATTATCCTGGCCAACAACAGCCTGAGCAGCAACGGCAATGTGACCGAAAGC |
| | GGCTGCAAGGAATGCGAGGAGCTCGAGGAGAAGAACATCAAGGAGTTCCTG |
| | CAGAGCTTCGTGCACATCGTGCAGATGTTCATCAACACCAGC |

In some embodiments, the polynucleotide encoding the present CAR or CCR is an mRNA molecule. The mRNA may further comprise a poly (A) sequence, e.g., a sequence encompassing 50-5000, 100-5000, 50-2000, 100-2000, 50-1000, or 100-1000 adenines.

### Pharmaceutical Compositions and Formulations

In accordance with the present invention, compositions comprising CB-NK cells expressing ADGRE2 targeting chimeric receptors, and nucleic acid molecules encoding the same are provided. The compositions contemplated herein may comprise one or more ADGRE2 targeting chimeric receptor polypeptides, polynucleotides, vectors comprising same, or genetically modified CB-NK cell as contemplated herein. Compositions include, but are not limited to pharmaceutical compositions.

In some embodiments, a composition comprises one or more cells modified to express one or more ADGRE2 targeting chimeric receptor polypeptides as described herein. In some embodiments, pharmaceutical compositions comprising an effective amount of cells, compositions comprising immune effector cells (e.g., NK cells) as described herein are provided. In some embodiments, a pharmaceutical composition comprises a pharmaceutically acceptable carrier, diluent or excipient and cells expressing a ADGRE2 targeting CAR polypeptide as contemplated herein.

The pharmaceutically acceptable carrier, diluent or excipient includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as com starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, com oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In some embodiments, pharmaceutical compositions comprise an effective amount of CAR-expressing immune effector cells (e.g., CB-NK cells) contemplated herein. As used herein, the term “an effective amount” refers to an amount effective of a genetically modified therapeutic cell, e.g., NK cell, to achieve a beneficial or desired prophylactic or therapeutic result, including clinical results (e.g., anti-cancer).

Pharmaceutical compositions comprising CB-NK cell populations modified to express a ADGRE2 targeting chimeric receptor of the present invention may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions are preferably formulated for parenteral administration, e.g., intravascular (intravenous or intraarterial), intraperitoneal or intramuscular administration.

The compositions may be liquid compositions. The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In some embodiments, the CB-NK cells discussed herein, and compositions contemplated herein are formulated in a pharmaceutically acceptable cell culture medium. Such compositions are suitable for administration to human subjects. In particular embodiments, the pharmaceutically acceptable cell culture medium is a serum free medium. Serum-free medium has several advantages over serum containing medium, including a simplified and better-defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

In other embodiments, compositions comprising immune effector cells as described herein are formulated in a solution comprising a cryopreservation medium. For example, cryopreservation media with cryopreservation agents may be used to maintain a high cell viability outcome post-thaw.

In some embodiments, compositions comprise an effective amount of immune effector cells modified to express a ADGRE2 targeting chimeric receptor polypeptide described herein, alone or in combination with one or more therapeutic agents. Thus, the chimeric receptor-expressing CB-NK cell compositions may be administered alone or in combination with other known cancer treatments, such as radiation therapy, chemotherapy, transplantation, immunotherapy, hormone therapy, photodynamic therapy, etc.

### Packages and Kits

Any of the compositions described herein may be comprised in a package and/or a kit for clinical use. In a non-limiting example, cells, reagents to produce cells, vectors, and reagents to produce vectors and/or components thereof may be comprised in a kit. In certain embodiments, NK cells may be comprised in a kit, and they may or may not yet express a ADGRE2-chimeric receptor comprising (a) CD28 hinge, an optional cytokine (e.g., IL-15), or an optional suicide gene. Such a kit may or may not have one or more reagents for manipulation of cells. Such reagents include small molecules, proteins, nucleic acids, antibodies, buffers, primers, nucleotides, salts, and/or a combination thereof, for example. Nucleotides that encode one or more chimeric receptors, suicide gene products, and/or cytokines may be included in the kit. Proteins, such as cytokines or antibodies, including monoclonal antibodies, may be included in the kit. Nucleotides that encode components of engineered chimeric receptors may be included in the kit, including reagents to generate same.

### Methods Use and Treatment

In some embodiments, the CB-NK cell comprising an ADGRE2 chimeric receptor described herein (e.g., a chimeric receptor comprising an ADGRE2 antigen binding domain as described herein) is used in a method of treating one or more ADGRE2-associated conditions. In some embodiments, the CB-NK cell comprising an ADGRE2 chimeric receptor is for use as a medicament. ADGRE2-associated conditions can include, without limitation, conditions that are caused by, include, include symptoms resulting in whole or in part from, or are known to occur in conjunction with ADGRE2 expression.

In some aspects, the present invention provides a method for treating a cancer comprising administering a CB-NK cell comprising an ADGRE2 chimeric receptor. A cancer is a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. In some embodiments, the “cancer” or “cancer tissue” comprises a solid tumor. Examples of cancers that can be treated by the methods of the present invention include, but are not limited to, cancers of the immune system including lymphoma, leukemia, myeloma, and other leukocyte malignancies. In some embodiments, the cancer is a acute myeloid leukemia.

In some embodiments, the lymphoma is selected from the group consisting of Acute Lymphoblastic Leukemia (ALL), AIDS-related lymphoma, ALK-positive large B-cell lymphoma, Burkitt's lymphoma, Chronic lymphocytic leukemia (CLL), Classical Hodgkin lymphoma, Diffuse large B-cell lymphoma (DLBCL), Follicular lymphoma, Intravascular large B-cell lymphoma, Large B-cell lymphoma arising in HHV8-associated multicentric Castleman's disease, Lymphomatoid granulomatosis, Lymphoplasmacytic lymphoma, Mantle cell lymphoma (MCL), Marginal zone B-cell lymphoma (MZL), Mucosa-Associated Lymphatic Tissue lymphoma (MALT), Nodal marginal zone B cell lymphoma (NMZL), Nodular lymphocyte predominant Hodgkin's lymphoma, Non-Hodgkin's lymphoma, Plasmablastic lymphoma, Primary central nervous system lymphoma, Primary effusion lymphoma, Splenic marginal zone lymphoma (SMZL), and Waldenstrom's macroglobulinemia. In some embodiments, the lymphoma is selected from the group consisting of Acute Lymphoblastic Leukemia (ALL), Chronic lymphocytic leukemia, CLL), Diffuse large B-cell lymphoma (DLBCL), Follicular lymphoma, Mantle cell lymphoma (MCL), Marginal zone B-cell lymphoma (MZL), Mucosa-Associated Lymphatic Tissue lymphoma (MALT), and Non-Hodgkin's lymphoma. In some embodiments, the lymphoma is Non-Hodgkin's lymphoma. In some embodiments, the cancer is relapsed and refractory acute myeloid leukemia.

In certain embodiments, the tumor is a cancer. In certain embodiments, the tumor is blood cancer. In certain embodiments, the tumor is selected from the group consisting of multiple myeloma, leukemia, lymphomas, and myeloid malignancies. Non-limiting examples of blood cancer include multiple myeloma, leukemia, and lymphomas. Non-limiting examples of leukemia include acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia. The lymphoma can be Hodgkin's lymphoma or non-Hodgkin's lymphoma. Non-limiting examples of myeloid malignancies include myelodysplastic syndromes (MDS), myeloproliferative neoplasms (MPN), myeloid/lymphoid neoplasms (e.g., myeloid/lymphoid neoplasms with eosinophilia and rearrangement of Platelet Derived Growth Factor Receptor Alpha (PDGFRA), Platelet Derived Growth Factor Receptor Beta (PDGFRB), or Fibroblast Growth Receptor 1 (FGFR1), or with PCM1-JAK2), acute myeloid leukemia (AML), blastic plasmacytoid dendritic cell neoplasm, B-lymphoblastic leukemia/lymphoma, and T-lymphoblastic leukemia/lymphoma. In certain embodiments, the myeloid malignancies comprises myelodysplastic syndromes.

In certain embodiments, the tumor is a B cell malignancy. Non-limiting examples of B cell malignancy include B cell lymphoma (BCL), B cell acute lymphocytic leukemia (ALL), B cell chronic lymphocytic leukemia (CLL), multiple myeloma (MM), CLL with Richter's transformation, and CNS lymphoma. B cell lymphoma includes B cell non-Hodgkin lymphoma (NHL) and B cell Hodgkin's lymphoma.

In various embodiments, administration of a CB-NK cell comprising an ADGRE2 chimeric receptor described herein results in a decrease in the prevalence, frequency, level, and/or amount of one or more symptoms or biomarkers of a ADGRE2-associated condition as described herein or otherwise known in the art, e.g., a decrease of at least about 3%, about 4%, about 5%, about 6%, about 7%, about 8%, about 9%, about 10%, about 15%, about 20%, about 25%, about 30%, about 35%, about 40%, about 45%, about 50%, about 60%, about 70%, about 80%, about 90%, 95%, about 99%, or about 100% of one or more symptoms or biomarkers as compared to a prior measurement in the subject or to a reference value.

In some embodiments, administration of a CB-NK cell comprising an ADGRE2 chimeric receptor to a subject having cancer results in a greater decrease or improvement in one or more symptoms or biomarkers of cancer than does a reference chimeric receptor expressing cell e.g., a cell that expresses an ADGRE2 chimeric receptor that cross-competes for ADGRE2 binding, under comparable conditions.

In some embodiments, a CB-NK cell comprising an ADGRE2 chimeric receptor (e.g., comprising an ADGRE2 antigen binding domain described herein) can be physically introduced to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, including a topical, epidermal or mucosal route of administration, for example, intranasally, vaginally, rectally, sublingually or topically. Administering can also be performed, for example, once, a plurality of times, and/or over one or more extended periods.

In another aspect of the present invention, methods, among other things, of use of ADGRE2 targeting, cells and compositions comprising the same are provided.

The ADGRE2 targeting chimeric receptors, and genetically modified CB-NK cells expressing a ADGRE2 targeting chimeric receptors contemplated herein provide improved methods of adoptive immunotherapy for use in the prevention, treatment, and amelioration of ADGRE2 related conditions. The genetically modified immune effector cells expressing a ADGRE2 targeting chimeric receptor described herein, provide improved methods of immunotherapy for use in increasing the cytotoxicity in cancer cells in a subject or for use in decreasing the number of cancer cells in a subject.

In some embodiments, a type of cellular therapy where NK cells are genetically modified to express the present chimeric receptor polypeptide that targets ADGRE2 expressing cancer cells, and the NK cells are infused to a recipient in need thereof is provided. The infused cell is able to kill disease causing cells, e.g., tumor cells, in the recipient. NK cells that express the present ADGRE2 targeting chimeric receptor can undergo robust in vivo cell expansion and can persist for an extended amount of time.

In some embodiments, methods of treating a cancer such as a ADGRE2 associated cancer in a subject in need are provided; the methods involve in administering to the subject in need a therapeutically effective amount of compositions as described in the present disclosure. The therapeutically effective amount of the composition comprising a genetically modified therapeutic cell may vary according to factors such as the disease state, age, sex, and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects.

In accordance with the present invention, it can generally be stated that a pharmaceutical composition comprising the CB-NK cells described herein may be administered at a dosage of 10⁴ to 10¹⁰ cells/kg body weight, preferably 10⁵ to 10⁶ cells/kg body weight, including all integer values within those ranges. The number of cells will depend upon the ultimate use for which the composition is intended as will the type of cells included therein. For uses provided herein, the cells are generally in a volume of a liter or less, 950 mL or less, 900 mL or less, 850 mL or less, 800 mL or less, 750 mL or less, 700 mL or less, 650 mL or less, 600 mL or less, 500 mLs or less, even 250 mLs or 100 mLs or less. Hence the density of the desired cells is typically greater than 10⁶ cells/ml and generally is greater than 10⁷ cells/ml, generally 10⁸ cells/ml, generally 10⁹ or greater. The clinically relevant number of immune cells can be apportioned into multiple infusions that cumulatively equal or exceed 10⁵, 10⁶, 10⁷, 10⁸ , 10⁹, 10¹⁰, 10¹¹, or 10¹² cells. In some aspects, particularly since all the infused cells will be redirected to a particular target antigen, lower numbers of cells, in the range of 10⁶/kilogram (10⁶-10¹¹ per patient) may be administered. Compositions may be administered multiple times at dosages within these ranges.

In some embodiments, the immune effector cells, e.g, NK cells that express a ADGRE2 targeting chimeric receptor of the present invention, are administered to a subject in need at a dose of at least 0.1×10⁵ cells, at least 0.5×10⁵ cells, at least 1×10⁵ cells, at least 5×10⁵ cells, at least 1×10⁶ cells, at least 0.5×10⁷ cells, at least 1×10⁷ cells, at least 0.5×10⁸ cells, at least 1×10⁸ cells, at least 0.5×10⁹ cells, at least 1×10⁹ cells, at least 2 x 10 9 cells, at least 3×10⁹ cells, at least 4×10⁹ cells, at least 5×10⁹ cells, or at least 1×10¹⁰ cells.

As non-limiting examples, the NK cells engineered to express a ADGRE2 targeting CAR of the present invention are administered at a dose of about 1×10⁶ to 2×10⁷, or about 1×10⁶ to about 1.5×10⁷, or about 1×10⁶ to 1×10⁷, or about 5×10⁶ to 2×10⁷, or about 5×10⁶ to 1.5×10⁷, or about 5×10⁶ to 1×10⁷ cells.

In some embodiments, the amount of CB-NK cells that express a ADGRE2 targeting chimeric receptor (e.g., CAR or CCR) of the present invention, are administered to a subject is at least 0.1×10⁴ cells/kg of body weight, at least 0.5×10⁴ cells/kg of body weight, at least 1×10⁴ cells/kg of body weight, at least 5×10⁴ cells/kg of body weight, at least 1×10⁵ cells/kg of body weight, at least 0.5×10⁶ cells/kg of body weight, at least 1×10⁶ cells/kg of body weight, at least 0.5×10⁷ cells/kg of body weight, at least 1×10⁷ cells/kg of body weight, at least 0.5×10⁸ cells/kg of body weight, at least 1×10⁸ cells/kg of body weight, at least 2×10⁸ cells/kg of body weight, at least 3×10⁸ cells/kg of body weight, at least 4×10⁸ cells/kg of body weight, at least 5×10⁸ cells/kg of body weight, at least 1×10⁹ cells/kg, or at least 1×10¹⁰ cells/kg of body weight. In particular embodiments, about 1×10⁶ NK cells/kg of body weight to about 1×10⁸ NK cells/kg of body weight, about 2×10⁶ NK cells/kg of body weight to about 0.9×10⁸ NK cells/kg of body weight, about 3×10⁶ NK cells/kg of body weight to about 0.8×10⁸ NK cells/kg of body weight, about 4×10⁶ NK cells/kg of body weight to about 0.7×10⁸ NK cells/kg of body weight, about 5×10⁶ NK cells/kg of body weight to about 0.6×10⁸ NK cells/kg of body weight, or about 5×10⁶ NK cells/kg of body weight to about 0.5×10⁸ NK cells/kg of body weight are administered to a subject.

In some embodiments, the amount of CB-NK cells that express a ADGRE2 targeting chimeric receptor (e.g., CAR or CCR) are administered to a subject is between about 20×10⁶ and about 150×10⁷ cells.

In some embodiments, the amount of CB-NK cells that express a ADGRE2 targeting chimeric receptor (e.g., CAR or CCR) are administered to a subject is about 20×10⁶, about 100×10⁶ or about 500×10⁶, or about 150×10⁷ cells.

It is recognizable that multiple administrations of the compositions contemplated herein may be required to affect the desired therapy. For example, a composition may be administered 1, 2, 3, 4, 5, 6, 7, 8, 9, 10, or more times over a span of 1 week, 2 weeks, 3 weeks, 1 month, 2 months, 3 months, 4 months, 5 months, 6 months, 1 year, 2 years, 5 years, 10 years, or during the life time of a subject in need.

The administration of the present compositions may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrastemal injection and infusion. In one embodiment, the compositions contemplated herein are administered to a subject by direct injection into a tumor, lymph node, or site of infection.

### Combination Therapies

The ADGRE2 targeting chimeric receptor and the immune effector cells expressing the same may be used in combination with other known agents and therapies. The ADGRE2 targeting chimeric receptor therapy and the at least one additional therapeutic agent can be administered simultaneously, in the same or in separate compositions, or sequentially. For sequential administration, the chimeric receptor -expressing cells described herein can be administered first, and the additional agent can be administered second, or the order of administration can be reversed. The administrations may be in intervals ranging from concurrently to minutes to days to weeks to months.

In some embodiments, the additional therapy is the administration of side-effect limiting agents (e.g., agents intended to lessen the occurrence and/or severity of side effects of treatment, such as anti-nausea agents, etc.).

In some embodiments, the additional therapy may be another specific anti-cancer therapy such as radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, hormone therapy, oncolytic viruses, or a combination of the foregoing.

A wide variety of chemotherapeutic agents may be used in combination with the present composition. A “chemotherapeutic agent” is used to connote a compound or composition that is administered in the treatment of cancer.

Additional immunotherapies may be used in combination or in conjunction with composition and methods described herein. Exemplary immunotherapeutic agents may include antibodies, antibody-drug conjugates, cancer vaccines, immune effector cells and immune checkpoint inhibitors.

The present compositions and methods may be used in combination with surgery. Approximately 60% of persons with cancer will undergo surgery of some type, which includes preventative, diagnostic or staging, curative, and palliative surgery. After and/or before surgery, a patient in need may be treated immune effector cells.

In some embodiments, other agents may be used in combination with certain aspects of the present embodiments to improve the therapeutic efficacy of treatment. Exemplary therapeutic agents that can be combined with the present composition may include small molecule enzymatic inhibitors, anti-metastatic agents, cytokines, growth factors, steroids, NSAIDs, DMARDs, anti-inflammatories, chemotherapeutics, radiotherapeutics, therapeutic antibodies, or other active and ancillary agents.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

The following examples describe some of the preferred modes of making and practicing the present invention. However, it should be understood that these examples are for illustrative purposes only and are not meant to limit the scope of the invention.

### Example 1

### ADGRE2 CAR or CCR CB-NK Cells Demonstrate In Vitro Cytotoxicity Activity

Cord blood (CB) units for research were obtained from the MD Anderson Cancer Center Cord Blood Bank. CB mononuclear cells were isolated from frozen CB units by Ficoll density gradient centrifugation. CB NK were then isolated using EasySep Human NK Isolation Kit (Stem Cell Technologies #17955). Isolated CB-NK cells were activated on Day 0 by co-culturing with irradiated uAPC cells and were grown in NK media [StemSpan™ SFEM II media (STEMCELL Technologies Cat #09655)+2 mM L-Glutamine (1% GIBCO L-Glutamine 200 mM REF 25030-081)+10% GIBCO Heat Inactivated Fetal Bovine Serum Certified One Shot™ Performance Plus (REF A38400-02)] and contained 200 IU/mL recombinant IL-2 (Miltenyi 130-097-743). During expansion cells were fed with fresh IL-2 every 2 days. On Day 6, cells were transduced with RD114 virus using spinoculation. Cells were stimulated with a second round of uAPC addition on Day 8 and fed IL-2 every 2 days until they were used for in vivo or in vitro studies on Day 15.

Acute myeloid leukemia (AML) cell lines, MOLM-13 and KG-la were transduced with virus carrying a firefly luciferase gene under a constitutive promoter and a selectable puromycin resistance gene. MV-4-11 cells were transduced with virus carrying a firefly luciferase gene and GFP gene under a constitutive promoter and a selectable puromycin resistance gene. The resulting cells were initially cultured in the presence of puromycin to select for a highly enriched population of luciferase-expressing cells. Luciferase-expres sing MOLM-13, KG-1a, and MV-4-11 cells were routinely cultured in the following media:

MOLM-13: GIBCO RPMI 1640 (REF 11875-093)+20% GIBCO Heat Inactivated Fetal Bovine Serum Certified One Shot™ Performance Plus (REF A38400-02)

KG-1a: GIBCO IMDM (REF 12440-053)+10% GIBCO Heat Inactivated Fetal Bovine Serum Certified One Shot™ Performance Plus (REF A38400-02)

MV-4-11: GIBCO IMDM (REF 12440-053)+10% GIBCO Heat Inactivated Fetal Bovine Serum Certified One Shot™ Performance Plus (REF A38400-02)

Cells growing in log phase at a typical density of 2-20×10⁶ live cells per mL were harvested by centrifugation at 350×g for 4 minutes. The supernatant was removed by aspiration and the cell pellet was resuspended in 10-20 mL of NK cell media which consisted of STEMCELL Technologies StemSpan™ SFEM II media (Cat #09655) with 2 mM L-Glutamine (1% GIBCO L-Glutamine 200 mM REF 25030-081) and 10% GIBCO Heat Inactivated Fetal Bovine Serum Certified One Shot™ Performance Plus (REF A38400-02), without IL-2. AML cell density and viability were counted using a Via1-Casette™ on a NucleoCounter NC200™ cell counting instrument. AML cells were then diluted with additional NK cell media to a live cell density of 333.3×10³ cells per mL. AML cells were dispensed using a MultiDrop™ Combi reagent dispenser with a standard dispensing cassette into Corning white 384-well flat-bottom poly-D-lysine-treated plates (#354661) to achieve a plating density of 10,000 live cells per well. The resulting plates were then incubated in a water-jacked incubator at 37° C. with 5% CO₂ atmosphere for 1-2 hours.

Transduced or untransduced Cord-blood NK effector cells were counted and diluted with NK cell media (without IL-2) to a live cell density of 3×10⁶ cells per mL. Effector cells were pipetted into a 384-well V-bottom polypropylene plate (Greiner, catalogue #781280) and serial diluted 1 in 3 in NK media (without IL-2) across for 4 point titration. Serial diluted effector cells were transfer to plate containing requisite AML cell line, with effector-to-target ratios (E:T) of 3:1, 1:1, 0.3:1 and 0.1:1 with final number of 30,000, 10,000, 3,333 and 1,111 effector cells, respectively. The resulting co-culture plate is then incubated at 37° C. with 5% CO₂ for 20-24 hours. Following incubation, Promega ONE-Glo™ Luciferase Assay System reagent (Promega #E6120) is added via a MultiDrop™ Combi to each plate.

The luminescent signal from each well is then determined using a BMG LABTECH PHERAstar FSX plate reader with a LUM Plus optics module. The signal from each well is normalized relative to the average signal of the AML cell line only controls. This normalized signal is then inverted to obtain a percent specific killing as determined by the decrease in AML luminescence.

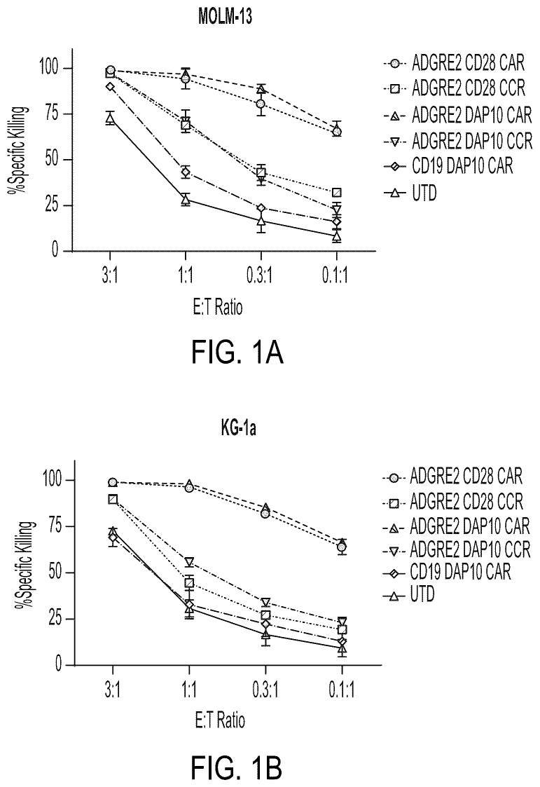

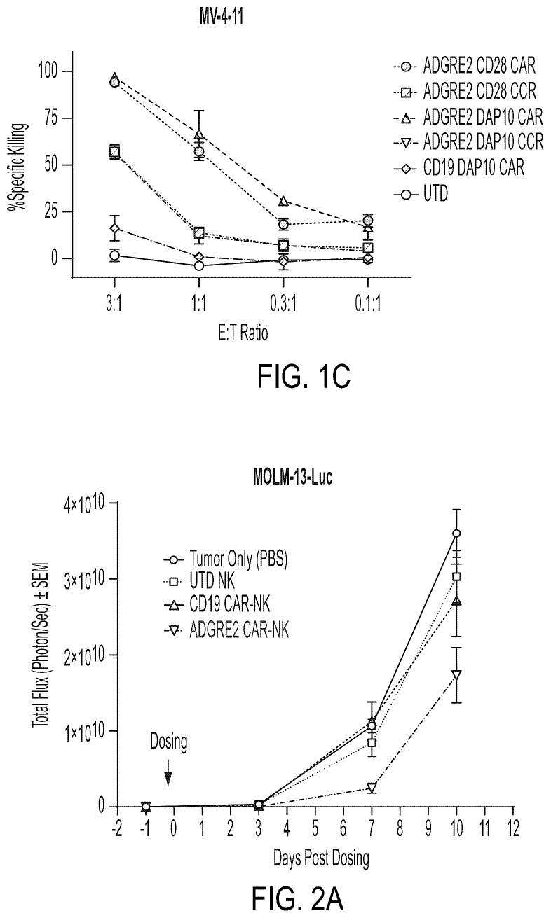

FIG. 1A-1C shows exemplary results of in vitro cytotoxicity activity of ADGRE2 CAR or CCR CB-NK against acute myeloid leukemia cell lines MOLM-13 (FIG. 1A), KG-1a (FIG. 1B), and MV-4-11 (FIG. 1C).

### Example 2

### ADGRE2 CAR CB-NK Cells Demonstrate In Vivo Efficacy

This example demonstrates efficacy of ADGRE2 CAR expressing CB-NK cells against acute myeloid leukemia cells in vivo.

NSG mice (NOD.Cg-Prkdcscid Il2rgtm1Wj1/SzJ) were purchased from The Jackson Laboratory. Female mice aged between 10 and 12 weeks old were used in this study. All animals were exposed to 1.5Gy of X-Ray whole body irradiation using Precision X-Ray SmART+ (North Branford, CT, USA) and 24 h later each animal intravenously (i.v.) injected with 0.5×10⁶ MOLM-13-RFluc, 5×10⁶ MV4-11-eGFP/1uc and 5×10⁶ KG-1a-1uc (cells in 200 μl PBS. The tumors were allowed to grow for 2 days (MOLM-13-RFluc), 3 days (MV4-11-eGFP/1uc), and 6 days (KG-1a-1uc) and randomized the mice into the groups (n=4 animals/group) using the individual whole body Bioluminescence imaging (BLI) signal and BW by the Studylog (Studylog Systems [Pacifica, CA, USA]) data acquisition and reporting system. On the day 3, 4 and 7 post-tumor (MOLM-13-RFluc, MV4-11-eGFP/1uc, and KG-1a-1uc, respectively) engraftments, mice were dosed with CAR expressing NK (i.e., CAR CB-NK) cells and untransduced NK (UTD-NK) in the relevant concentrations in 200 μL PBS/animal by tail i.v. injection. To observe the tumor burden and efficacy of the test articles, mice were imaged twice weekly (MOLM-13-RFluc model) and once weekly (MV4-11-eGFP/1uc and KG-1a-1uc model) and BW were taken twice weekly. On the day of imaging, mice were injected intraperitoneally (IP) with 200 μL RediJect D-Luciferin (150 mg/kg BW) (Perkin Elmer, MA, USA) and subjected to IVIS® SpectrumCT (Perkin Elmer, MA) in the ventral position. Total flux (photons/second) for the entire mouse was quantified using Living Image software (PerkinElmer, USA). The data were analyzed and expressed as Mean ±SEM (n=4) using Graph-Pad Prism 8.

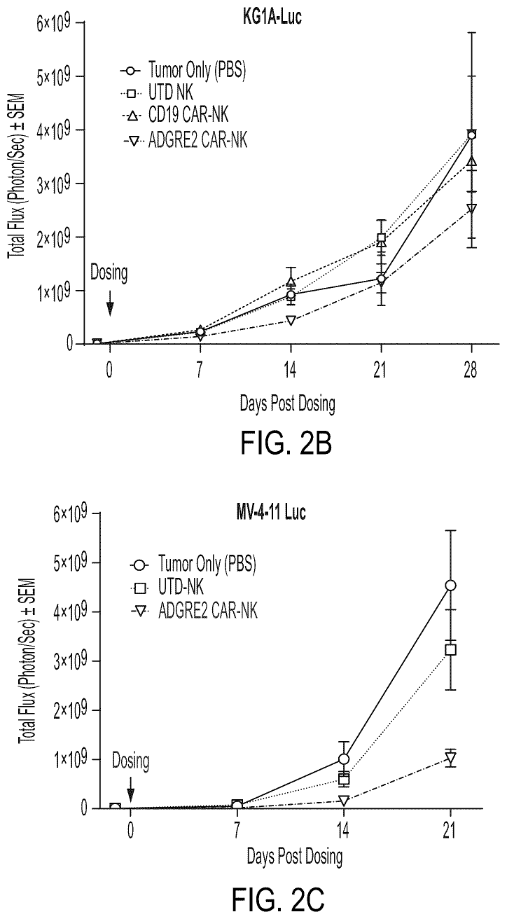

FIG. 2A-2C shows exemplary results of in vivo efficacy of ADGRE2 CAR CB-NK against acute monocytic leukemia cell lines MOLM-13 (FIG. 2A), KG-1a (FIG. 2B), and MV-4-11 (FIG. 2C).

### Example 3

### OX40 Co-Stimulatory Domain CCR

This Example probed the effect of exemplary costimulatory domains in CCR constructs (chimeric receptors without an intracellular CD3z domain) using an in vitro Repeat Antigen Stimulatory Assay.

Cord blood (CB) units for research were obtained from the MD Anderson Cancer Center Cord Blood Bank. CB mononuclear cells were isolated from frozen CB units by Ficoll density gradient centrifugation. CB NK cells were then isolated using EasySep Human NK Isolation Kit (Stem Cell Technologies #17955). Isolated CB-NK cells were activated on Day 0 by co-culturing with irradiated uAPC cells and were grown in NK media [StemSpan™ SFEM II media (STEMCELL Technologies Cat #09655)+2 mM L-Glutamine (1% GIBCO L-Glutamine 200 mM REF 25030-081)+10% GIBCO Heat Inactivated Fetal Bovine Serum Certified One Shot™ Performance Plus (REF A38400-02)] and contained 200 IU/mL recombinant IL-2 (Miltenyi 130-097-743). During expansion cells were fed with fresh IL-2 every 2 days. On Day 6, cells were transduced with RD114 virus using spinoculation. CD123 tool binder 26292 was used for each design. Cells were stimulated with a second round of uAPC addition on Day 8 and fed IL-2 every 2 days until they were used for in vivo or in vitro studies on Day 15.

Acute myeloid leukemia (AML) cell line, MOLM-13 was transduced with virus carrying a firefly luciferase gene under a constitutive promoter and a selectable puromycin resistance gene. The resulting cells were initially cultured in the presence of puromycin to select for a highly enriched population of luciferase-expressing cells. Luciferase-expressing MOLM-13 cells were routinely cultured in the following media:

MOLM-13: GIBCO RPMI 1640 (REF 11875-093)+20% GIBCO Heat Inactivated Fetal Bovine Serum Certified One Shot™ Performance Plus (REF A38400-02)

MOLM-13 cells growing in log phase at a typical density of 2-20×106 live cells per mL were harvested by centrifugation at 350×g for 4 minutes. The supernatant was removed by aspiration and the cell pellet was resuspended in 10-20 mL of NK cell media without IL-2. AML cell density and viability were counted using a Via1-Casette™ on a NucleoCounter NC-200™ cell counting instrument. AML cells were then re-suspended in NK cell media containing 2× concentrations of recombinant IL-2 (200 IU/mL) (Miltenyi 130-097-743) and recombinant IL-15 (2 ng/mL) (Peprotech 200-15-10UG) to make a live cell density of 4×105 cells per mL. 100 uL of AML cells (40,000 total per well) were dispensed using a MultiDrop™ Combi reagent dispenser with a standard dispensing cassette into Corning white 96-well flat-bottom non-cell culture treated sterile plates. The resulting plates were then incubated in a water-jacked incubator at 37° C. with 5% CO2 atmosphere for 2-3 hours.

Transduced or untransduced CB-NK effector cells were counted and diluted with NK cell media (without IL-2/15) to a live cell density of 4×105 cells per mL. 100 uL of effector cells (40,000 total per well) were pipetted into a 96-well plate containing AML for a 1:1 E:T ratio. Co-culture plates were placed in a 37 ° C. 5% CO2 humidified incubator.

On Days 2, 5, 7, 9, 12, and 14 post initiation of co-culture, the effector-target co-culture plates are removed from the incubator and shaken briefly using a MultiDrop Combi. A Dynamic Devices Lynx liquid handler with 96SV head and 200 uL Filtered Tip Racks (one rack per co-culture plate) is used mix each well to fully resuspend target and effector cells. Next a sample of 20 uL cell suspension from each plate is transferred into a 384-well V-bottom Polypropylene plate (Greiner) to flow cytometry analysis. To each well, FACS stain mix (1:75 BioLegend anti-Nectin-2-PE (1:125 final, Clone TX31) and 1:75 BioLegend anti-CD56-B V711 (1:125 final, Clone HCD56). is added. Stain mix is incubated 30 minutes at 4 deg C. After 30 min, cells are the washed FACS buffer. After wash, sytox blue stain mixture (1:1000; Invitrogen) is added to each well and plate is read on the Novocyte Advanteon. After sample is withdrawn for FACS analysis, the remaining NK cells are re-challenged by centrifuging and removing supernatant and adding 40,000 fresh AML cells in NK media containing 100IU/mL IL-2 and 1 ng/mL IL-15. The plates are then returned to 37 ° C. 5% CO2 incubation until the next time point.

For flow cytometry analysis, compensation was performed using UltraComp Plus eBeads (Invitrogen) for anti-CD56-BV711 and anti-Nectin-2-PE and cells were used for sytox blue. Each sample is gated to exclude cell debris using SSC-H vs FSC-H and then gated to exclude cell clumps and doublets using FSC-A vs FSC-H. Next FSC-H vs Sytox Blue-H is used to gate for live cells. For wells containing MOLM-13 target cells, live cells are gated using anti-Nectin-2-PE-H vs FSC-H where large and Nectin-2+ cells are gated as target and small and Nectin-2− cells are gated as effector. The number of live target cells and live effector cells at each time point is quantitated and ability to continue to control tumor at each subsequent challenge is monitored over time.

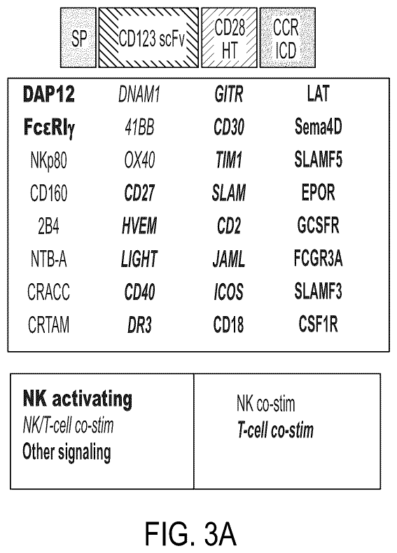

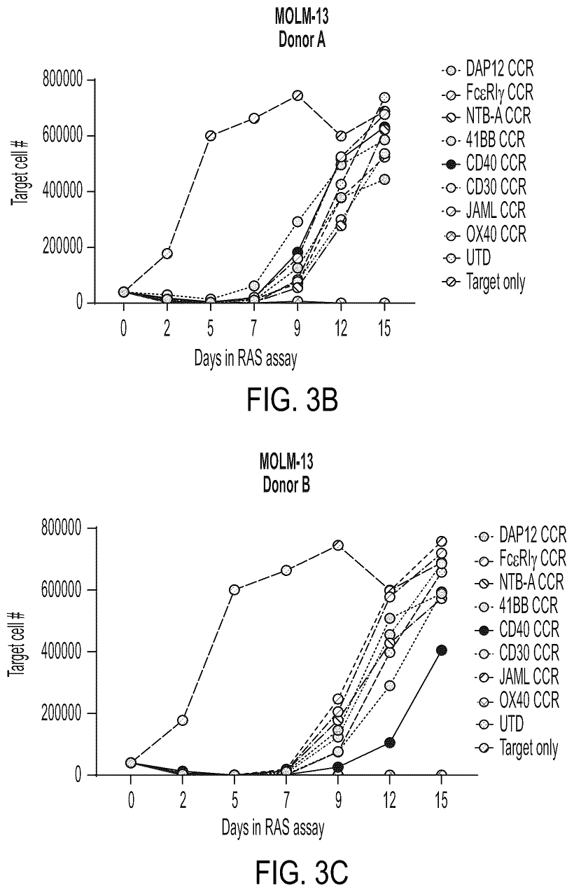

FIG. 3A shows co-stimulatory molecules that were screened in chimeric receptor constructs (constructs without the intracellular CD3z). FIG. 3B and FIG. 3C exemplify the ability of NK cells engineered with OX40-CCR to durably control MOLM-13 tumor cells across many different tumor re-challenges. OX40 costimulatory CCR demonstrated potent control of MOLM-13 tumor cells.

### Other Embodiments

While a number of embodiments of this invention are described herein, the present disclosure and examples may be altered to provide other methods and compositions of this invention. Therefore, it will be appreciated that the scope of this invention is to be defined by the appended claims in addition to the specific embodiments that have been represented by way of example. All references cited herein are hereby incorporated by reference.

1. A cord blood natural killer (CB-NK) cell, comprising a chimeric receptor comprising an extracellular antigen-binding domain that binds to ADGRE2, a transmembrane domain, and an intracellular domain, wherein the extracellular antigen-binding domain comprises
a heavy chain variable region comprises a HCDR1 comprising an amino acid sequence of GYTFTNYW (SEQ ID NO: 1), a HCDR2 comprising an amino acid sequence of VYPGDGDT (SEQ ID NO: 2) and a HCDR3 comprising an amino acid sequence of ARGFTAYGMDY (SEQ ID NO: 3); and
a light chain variable region comprises a LCDR1 comprising an amino acid sequence of SSVSY (SEQ ID NO: 4), a LCDR2 comprising an amino acid sequence of DTS (SEQ ID NO: 5), and a LCDR3 comprising an amino acid sequence of QQWSSNPLT (SEQ ID NO: 6).

2. The CB-NK cell of claim 1, wherein the extracellular antigen-binding domain comprises a heavy chain variable region that is at least 90%, at least 91%, at least 92%, at least 93%, at least 94%, at least 95%, at least 96%, at least 97%, at least 98%, at least 99%, or 100% identical to the amino acid sequence set forth in SEQ ID NO: 7, 9 or 15.

3. The CB-NK cell of claim 1, wherein the extracellular antigen-binding domain comprises a light chain variable region that is at least 90%, at least 91%, at least 92%, at least 93%, at least 94%, at least 95%, at least 96%, at least 97%, at least 98%, at least 99%, or 100% identical to the amino acid sequence set forth in SEQ ID NO: 14, 12, or 16.

4-6. (canceled)

7. The CB-NK cell of claim 1, wherein the extracellular antigen-binding domain comprises a single chain variable fragment (scFv).

8. The CB-NK cell of claim 1, wherein the extracellular antigen-binding domain comprises a linker between the heavy chain variable region and the light chain variable region.

9. The CB-NK cell of claim 8, wherein the linker comprises the amino acid sequence of SEQ ID NO: 24.

10. (canceled)

11. The CB-NK cell of claim claim 1, wherein the extracellular antigen-binding domain comprises an scFv that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence set forth in SEQ ID NO: 19, 20 and 68.

12. The CB-NK cell of claim 1, wherein the transmembrane domain comprises a CD8 polypeptide, a CD28 polypeptide, a CD3s polypeptide, a CD4 polypeptide, a 4-IBB polypeptide, an OX40 polypeptide, an ICOS polypeptide, a CTLA-4 polypeptide, a PD-I polypeptide, a LAG-3 polypeptide, a 2B4 polypeptide, or a BTLA polypeptide.

13-14. (canceled)

15. The CB-NK cell of claim 1, wherein the intracellular domain further comprises at least one co-stimulatory signaling region.

16-27. (canceled)

28. The CB-NK cell of claim 1, comprising a nucleic acid encoding the chimeric receptor.

29-30. (canceled)

31. The CB-NK cell of claim 28, wherein the nucleic acid further comprises a promoter that is operably linked to the chimeric receptor.

32. (canceled)

33. The CB-NK cell of claim 31, wherein the promoter is selected from the group consisting of an elongation factor (EF)-1 promoter, a cytomegalovirus immediate-early promoter (CMV) promoter, a simian virus 40 early promoter (SV40) promoter, a phosphoglycerate kinase (PGK) promoter, a metallothionein promoter, Ubiquitin C promoter, a NFAT transcriptional response element (TRE) promoter, a CD69 promoter, a CD25 promoter, an IL-2 promoter, a 4-1BB promoter, a PD1 promoter, a LAG3 promoter, a TCR alpha promoter, a TCR beta promoter, and a beta 2-microglobulin promoter.

34-37. (canceled)

38. The CB-NK cell of claim 1, wherein the CB-NK cell further expresses exogenous IL-15.

39. A composition comprising the CB-NK cell of claim 1 and a pharmaceutically acceptable carrier.

40-42. (canceled)

43. A method of reducing tumor burden in a subject, comprising administering to the subject the CB-NK cell of claim 1.

44. (canceled)

45. A method of increasing or lengthening survival of a subject having a tumor, comprising administering to the subject the CB-NK cell of claim 1.

46. A method of treating or preventing a tumor in a subject, comprising administering to the subject the CB-NK cell of claim 1.

47. (canceled)

48. The method of claim 43, wherein the tumor is cancer.

49-57. (canceled)

58. A cord blood natural killer (CB-NK) cell comprising a nucleic acid encoding a chimeric receptor and an exogenous IL-15 polypeptide, wherein the chimeric receptor comprises
an extracellular antigen-binding domain that binds to ADGRE2 comprising a heavy chain variable region comprises a HCDR1 comprising an amino acid sequence of GYTFTNYW (SEQ ID NO: 1), a HCDR2 comprising an amino acid sequence of VYPGDGDT (SEQ ID NO: 2) and a HCDR3 comprising an amino acid sequence of ARGFTAYGMDY (SEQ ID NO: 3); and a light chain variable region comprises a LCDR1 comprising an amino acid sequence of SSVSY (SEQ ID NO: 4), a LCDR2 comprising an amino acid sequence of DTS (SEQ ID NO: 5), and a LCDR3 comprising an amino acid sequence of QQWSSNPLT (SEQ ID NO: 6).

59. A nucleic acid encoding a chimeric receptor and an exogenous IL-15 polypeptide, wherein the chimeric receptor comprises
an extracellular antigen-binding domain that binds to ADGRE2 comprising a heavy chain variable region comprises a HCDR1 comprising an amino acid sequence of GYTFTNYW (SEQ ID NO: 1), a HCDR2 comprising an amino acid sequence of VYPGDGDT (SEQ ID NO: 2) and a HCDR3 comprising an amino acid sequence of ARGFTAYGMDY (SEQ ID NO: 3); and
a light chain variable region comprises a LCDR1 comprising an amino acid sequence of SSVSY (SEQ ID NO: 4), a LCDR2 comprising an amino acid sequence of DTS (SEQ ID NO: 5), and a LCDR3 comprising an amino acid sequence of QQWSSNPLT (SEQ ID NO: 6).
